(19) United States
(12) Patent Application Publication
(10) Pub. No.: US 2021/0290753 A1
(43) Pub. Date: Sep. 23, 2021

(54) CRIMEAN-CONGO HEMORRHAGIC FEVER VIRUS IMMUNOGENIC COMPOSITIONS

(71) Applicant: UNIVERSITÉ LAVAL, Québec (CA)

(72) Inventors: Gary KOBINGER, Québec (CA); George BABUADZE, Toronto (CA)

(21) Appl. No.: 17/261,055

(22) PCT Filed: Nov. 8, 2019
(86) PCT No.: PCT/CA2019/051592

§ 371 Date: Jan. 18, 2021

Related U.S. Application Data
(60) Provisional application No. 62/850,895, filed on May 21, 2019.

(51) Int. Cl. A61K 39/12; A61P 37/04; C12N 7/00; A61P 31/14; A61K 39/00

(52) U.S. Cl. CPC A61K 39/12; A61P 37/04; C12N 7/00; C12N 2760/12022; C12N 2760/12034; A61K 2039/552; A61K 2039/53; A61P 31/14

(57) ABSTRACT

The present disclosure relates to DNA vaccines against Crimean-Congo Hemorrhagic Fever (CCHF) virus. The present disclosure also relates to CCHF virus sequences and their use for vaccination such as DNA vaccination. The present disclosure more particularly relates to artificial nucleic acid molecules that are able to encode a CCHF polypeptide of an infectious CCHF virus, a fragment or a variant thereof. The nucleic acid molecules of the present application are formulated as immunogenic compositions.

### TECHNICAL FIELD

The present disclosure relates to DNA vaccines against Crimean-Congo Hemorrhagic Fever (CCHF) virus. The present disclosure also relates to the CCHF virus sequences and their use for vaccination such as DNA vaccination. The present disclosure more particularly relates to artificial nucleic acid molecules that are able to encode a CCHF polypeptide of an infectious CCHF virus, a fragment or a variant thereof. The nucleic acid molecules of the present application are formulated as immunogenic compositions.

### BACKGROUND

DNA vaccines have recently deserved high interest. DNA vaccination relies on administration of DNA vectors encoding an antigen, or multiple antigens, for which an immune response is sought into a host. DNA vectors include elements that allow expression of the protein by the host's cells, and includes a strong promoter, a poly-adenylation signal and sites where the DNA sequence of the transgene is inserted. Vectors also contain elements for their replication and expansion within microorganisms. DNA vectors can be produced in high quantities over a short period of time and as such they represent a valuable approach in response to outbreaks of new pathogens. In comparison with recombinant proteins, whole-pathogen, or subunit vaccines, methods for their manufacturing are relatively cost-effective and they can be supplied without the use of a cold chain system.

DNA vaccines have been tested in animal disease models of infection, cancer, allergy and autoimmune disease. They generate a strong humoral and cellular immune response that has generally been found to protect animals from the disease.

Several DNA vaccines have been tested in human clinical trials including DNA vaccines for Influenza virus, Dengue Virus, Venezuelan Equine Encephalitis Virus, HIV, Hepatitis B Virus, Plasmodium Falciparum Malaria, Herpes Simplex, Zika virus etc. (Tebas, P. et al., N Engl J Med, 2017 (DOI: 10.1056/NEJMoa1708120); Gaudinski, M. R. et al., Lancet, 391:552-62, 2018).

The potency of DNA vaccines has been improved with the advent of new delivery approaches and improvements in vector design.

A number of technical improvements are being explored, such as gene optimization strategies, improved RNA structural design, novel formulations and immune adjuvants, and various effective delivery approaches. DNA based vaccines offers a number of potential advantages over traditional approaches, including the stimulation of both B- and T-cell responses, improved stability and the absence of infectious agent.

Several DNA vectors are under development for a variety of infectious agents including influenza virus, hepatitis B virus, human immunodeficiency virus, rabies virus, lymphocytic chorio-meningitis virus, malarial parasites and mycoplasmas. However, in spite of good humoral or cellular responses the protection from disease in animals has been obtained only in some cases.

The inventors have generated DNA vaccines expressing CCHF antigens. These antigens are expressed from vectors that show efficient transgene expression.

### SUMMARY

The present disclosure relates to Crimean-Congo Hemorrhagic Fever (CCFH) virus sequences and their use for vaccination such as DNA vaccination. The CCFH virus sequences of the present disclosure include full-length nucleic acid molecules, nucleic acid fragments and nucleic acid variants.

Nucleic acid molecules that are particularly contemplated by the present disclosure are those that encode a CCHF polypeptide (e.g., glycoprotein), a fragment or a variant.

Aspects and embodiments of the disclosure more particularly relate to artificial nucleic acid molecules that are able to encode a CCHF polypeptide of an infectious CCHF virus, a fragment or a variant thereof.

Exemplary embodiments of a CCHF polypeptide of an infectious CCHF virus includes without limitation a polypeptide having the amino acid sequence set forth in SEQ ID NO:7, in amino acid residues 77 to 720 of SEQ ID NO: 8, in amino acid residues 77 to 364 of SEQ ID NO: 9, in SEQ ID NO:10 or in SEQ ID NO: 15.

Since the CCHF polypeptide sequences disclosed herein are only representative examples, the nucleic acid molecules of the present disclosure may be modified so as to encode other CCHF polypeptides of infectious CCHF viruses. Exemplary embodiments of CCHF polypeptides of infectious CCHF viruses include those encoded by the nucleic acids set forth in Accession No. DQ019222.1 (representative example of Clade I), DQ211626.1 (representative example of Clade II), AY900141.1 (representative example of Clade III), AB069669.1 (representative example of Clade IV), AY675511.1 (representative example of Clade V), and DQ211628.1 (representative example of Clade VI) or any virus isolates thereof.

The nucleic acid molecules of the present disclosure may comprise, for example, the nucleic acid sequence set forth in SEQ ID NO: 3, a nucleic acid sequence corresponding to nucleotides 229 to 2163 of SEQ ID NO:4, a nucleic acid sequence corresponding to nucleotides 229 to 1092 of SEQ ID NO:5, the nucleic acid sequence set forth in SEQ ID NO:6 or the nucleic acid sequence set forth in SEQ ID NO:14.

More particularly contemplated are nucleic acid molecules that comprises the sequence set forth in SEQ ID NO:3, in SEQ ID NO:14, as well as variants and fragments thereof.

Exemplary embodiments of nucleic acid variants include for example, those that have a sequence at least 70% identical, at least 75% identical, at least 80% identical, at least 85% identical, at least 90% identical, at least 95% identical or at least 99% identical to SEQ ID NO: 3, to nucleotides 229 to 2163 SEQ ID NO:4, to nucleotides 229 to 1092 of SEQ ID NO:5, to SEQ ID NO:6 or to SEQ ID NO:14. Such variants may encode a CCHF polypeptide of infectious CCHF viruses or a fragment thereof.

The nucleic acid variant may have one or more codon that is replaced by an alternative codon in comparison with an original sequence and may encode the same amino acid as that of the original sequence. For example, the sequence of the nucleic acid variant may include one nucleotide difference in one or in several codons (e.g. each codon) of the original sequence provided that it encodes the same amino acid sequence.

As such, the nucleic acid variant of the present disclosure may encode the polypeptide set forth in SEQ ID NO:7, the polypeptide set forth in amino acid residues 77 to 720 of SEQ ID NO: 8, the polypeptide set forth in amino acid residues 77 to 364 of SEQ ID NO: 9, the polypeptide set forth in SEQ ID NO: 10 or the polypeptide set forth in SEQ ID NO: 15.

In another exemplary embodiment the nucleic acid variant may have one or more codons replaced by an alternative codon encoding a conservative amino acid substitution in comparison with the corresponding amino acid residue encoded by the original sequence thereby encoding a polypeptide variant.

In another exemplary embodiment the nucleic acid variant may comprise one or more codons that is replaced by an alternative codon encoding a non-conservative amino acid substitution in comparison with the corresponding amino acid residue encoded by the original sequence thereby encoding a polypeptide variant.

In accordance with the present disclosure, the polypeptide variant may be for example an immunogenic variant.

The nucleic acid variant of the present disclosure may therefore encode a polypeptide variant having at least 70%, at least 75%, at least 80%, at least 85%, at least 90%, at least 95% or at least 99% identity with SEQ ID NO:7, amino acid residues 77 to 720 of SEQ ID NO: 8, amino acid residues 77 to 364 of SEQ ID NO: 9, SEQ ID NO: 10 or SEQ ID NO: 15.

The choice of codon sequence may be based on its frequency in human where a codon having an increased frequency may be preferred. Particular codon sequence may also be selected so as to increase or decrease the G/C content of the original sequence. Codon modification is discussed for example in publication No. WO2019/038332, the entire content of which is incorporated herein by reference.

Nucleic acid fragments are also encompassed by the present disclosure. Such nucleic acid fragments may encode a polypeptide fragment or an immunogenic fragment.

Generally, a polypeptide fragment of about 8 to 10 amino acid residues (encoded by a nucleic acid fragment of at least about 24 to 30 nucleotide) will fit into a major histocompatibility (MHC) Class I molecule, whereas a polypeptide fragment of about 15 to 24 amino acid residues (encoded by a nucleic acid fragment of at least about 45 to 72 nucleotides) will fit into a MHC Class II molecule.

The present disclosure therefore relates to nucleic acid fragments encompassing at least 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72 consecutive nucleotides of the nucleic acid sequence set forth in SEQ ID NO:3, of the nucleic acid sequence set forth in nucleotides 229 to 2163 of SEQ ID NO:4, of the nucleic acid sequence set forth in nucleotides 229 to 1092 of SEQ ID NO:5, of the nucleic acid sequence set forth in SEQ ID NO:6 or of the nucleic acid sequence set forth in SEQ ID NO:14 and that encodes polypeptide fragment or an immunogenic fragment.

The present disclosure also relates to nucleic acid fragments encompassing at least 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59. 60, 61, 62, 63, 64, 65, 66, 67, 68, 69, 70, 71, 72 consecutive nucleotides of a nucleic acid variant having at least 70%, at least 75%, at least 80%, at least 85%, at least 90%, at least 95% or at least 99% identity with SEQ ID NO:3, with nucleotides 229 to 2163 of SEQ ID NO:4, with nucleotides 229 to 1092 of SEQ ID NO:5, with SEQ ID NO:6 or with SEQ ID NO:14 and that encodes polypeptide fragment, an immunogenic fragment or an immunogenic variant.

It is believed that Gn is more immunogenic than Gc and that the entire M segment is more immunogenic than the Gn or Gc segments and therefore nucleic acid fragments of the present invention may encompass Gn or a portion thereof. Nevertheless, Fritzen, A et al., (Epitope-mapping of the glycoprotein from Crimean-Congo hemorrhagic fever virus using a microarray approach; PLOS Neglected Tropical Diseases 12(7):e0006598, 2018) showed a positive immune response towards some particular CCHF glycoprotein fragments.

Exemplary embodiments of such polypeptide fragments include at least amino acid residues 231 to 250, amino acid residues 541 to 560, amino acid residues 551 to 570, amino acid residues 771 to 790, amino acid residues 951-970, amino acid residues 954-973, amino acid residues 951-975, amino acid residues 1041 to 1060, amino acid residues 1061 to 1080 and amino acid residues 1551 to 1570 of a CCHF glycoprotein precursor (exemplary embodiments of which are provided in SEQ ID NO:7 and SEQ ID NO:15). Nucleic acid fragments of the present disclosure include those that encode a sequence that consists of these polypeptide fragments.

Alternatively, the nucleic acid fragments of the present disclosure may include a sequence that encodes at least 20 amino acid residues that comprises CCHF polypeptide fragments. The CCHF polypeptide fragments may be used to make a fusion protein (e.g., with ubiquitin, albumin, KHL etc.) so as to increase the immune response.

In accordance with the present disclosure, the nucleic acid fragments may encode from 20 to 25, from 20 to 30, from 20 to 35, from 20 to 40, from 20 to 50, from 20 to 60, from 20 to 70, from 20 to 80, from 20 to 90, from 20 to 100, from 20 to 150, from 20 to 200, from 20 to 250, from 20 to 300, from 20 to 350, from 20 to 400, from 20 to 400, from 20 to 450, from 20 to 500, from 20 to 600, from 20 to 700, from 20 to 800, from 20 to 900, from 20 to 1000, from 20 to 1100, from 20 to 1200, from 20 to 1300, from 20 to 1400, from 20 to 1500, from 20 to 1600, from 20 to 1700 amino acids of a CCHF glycoprotein precursor.

It is to be understood herein that terms such as “from 20 to 1700” include any individual values comprised within and including 20 and 1700. Terms such as “from 20 to 1700” also include any individual sub-ranges comprised within and including from 20 to 1700, from 20 to 1680, from 20 to 1685, from 20 to 1687 etc.

The same definition applies for similar expressions written in the format “from about X to about Y”.

In some aspects of the disclosure, the sequence of the nucleic acid molecules of the present disclosure may be identical to that of naturally occurring nucleic acid molecules.

In other aspects of the disclosure, the sequence of the nucleic acid molecules of the present disclosure is not identical to that of naturally occurring nucleic acid molecules.

The nucleic acid molecules of the present disclosure may be single-stranded or double-stranded. The nucleic acid molecules disclosed herein may comprises deoxyribonucleotides, ribonucleotides, modified deoxyribonucleotides or modified ribonucleotides. The nucleic acid molecules of the present disclosure may comprise DNA or RNA.

The nucleic acid molecules of the present disclosure may be part of an expression cassette that comprises for example, regulatory sequences that control their expression (e.g., promoter, enhancer, a 3′-untranslated region, posttranscriptional regulatory elements and the like).

The nucleic acid molecules of the present disclosure may be incorporated into a vector suitable for its expression.

The CCHF virus sequences are cloned into vectors suitable for expressing transgenes. The DNA vector of the present disclosure may be used to express CCHF antigens into a host's cells and to trigger an immune response towards an antigenic portion of the proteins or peptides in a mammal.

Therefore, in additional aspects and embodiments, the present disclosure also relates to vectors that comprises a nucleic acid encoding a CCHF polypeptide. The vector may also encode other antigenic sequences. In addition, the present disclosure relates to a set of vectors wherein one vector comprises a nucleic acid encoding a CCHF polypeptide and the other vector comprises a nucleic acid encoding the other antigenic sequence (e.g., another CCHF antigen).

The nucleic acid molecules of the present disclosure may be expressed from the DNA vectors disclosed herein and especially from the pIDV-II vector such as to elicit an immune response towards a naturally occurring CCHF polypeptide.

Viral vectors are also suitable for vaccination. Such viral vectors are encompassed by the present disclosure and include for example viral genome composed of DNA. Suitable viral vectors are preferably replication defective, and include but are not limited to adenovirus; herpes virus; lentivirus; retrovirus; parvovirus, etc. As used herein the term “viral vector” refers to a viral genome that comprises the nucleic acid described herein or to viral particles containing same.

The vectors of the present disclosure can be formulated with a physiologically acceptable carrier for use in DNA vaccination.

The nucleic acid or vector of the present disclosure may be used as a naked vaccine or may be encapsulated into nanocarrier. The nanocarrier may be preferably biocompatible.

Exemplary embodiments of nanocarrier includes for example and without limitation, lipid-based nanocarriers such as those including cationic lipids, polymeric nanocarriers such as those including polyethylene glycol (PEG), modified PEG, PLG, PLGA. Poly-L_Lysine, polyethilenimine and the like and protein-based nanocarriers such as those including gelatin, albumin or viral-like particles.

In an exemplary embodiment, nanocarriers can be modified to incorporate elements that facilitate targeting to specific cell types such as for example antibodies or natural ligands such as carbohydrates.

The present disclosure relates to DNA vaccines comprising a CCHF virus sequence disclosed herein.

### BRIEF DESCRIPTION OF THE DRAWINGS

FIG. 1: picture of a Western blot under non-reduced conditions with anti-CCHFV monoclonal antibody −11E7 (used against the Gn protein of entire GP) as shown by a single protein expression of approximately 75 kDa; a) pIDV-II-CCHF-GP-Turkey (SEQ ID NO:26), b) pVAX1-CCHF-GP-Turkey and c) pCAGGS-CCHF-GP-Turkey Transfection in 293-LTV cells. 6 well plates. 300.000 cells/well, 5 μg DNA/well. Cell lyses with non-reduced condition lyses buffer. Western blot: 24 h after transfection. Proteins were quantified and ≈15 ug cell lysate+loading buffer was loaded into the blotting gel. Primary antibody: monoclonal anti-GP CCHF 11E7 dilution −1/2000. Secondary 1:20000 of secondary anti-a-Tubulin antibody and anti-mouse IgG, dilution −1/10000. CCHF GP of approximately 75 kDa (arrow), confirming recombinant protein expression. A loading control (lane 2) of 50 kDa shows an equal amount of loaded proteins.

FIG. 2: graph showing IFN-g ELISpot responses from Balb/c mice immunized with pIDV—II-CCHF-GP-Turkey or pVAX1-CCHF-GP-Turkey. Asterisks indicate statistically significant differences (****, p<0.005).

FIG. 3: graph showing CCHFV-specific IgG following immunization with pIDV—II-CCHF-GP-Turkey or with pVAX1-CCHF-GP-Turkey. *Two-way ANOVA, confidence intervals were set to 95%, P-value=<0.0001.

FIG. 4A and FIG. 4B: graphs showing CCHF-specific IgGs as measured by ELISA in sheep vaccinated with CCHFV-M DNA (G1) compared to sheep administered with buffer (control group) (FIG. 4A: individual sheep data; FIG. 4B: grouped data).

FIG. 5a-f: alignment of exemplary vectors pIDV-I and pIDV-II sequence used for expressing CCHF.

### DETAILED DESCRIPTION

### Definitions

As used herein the terms “vector” and “plasmid” are used interchangeably.

As used herein the term “vector backbone” refers to the vector portion of a given vector into which the sequence of a transgene has been cloned.

The term “transgene” refers to a gene encoding the protein(s) or peptide(s) of interest inserted in the vector of the present disclosure.

As used herein the term “90% sequence identity”, includes all values contained within and including 90% to 100%, such as 91%, 92%, 92.5%, 95%, 96.8%, 99%, 100%. Likely, the term “at least 75% identical” includes all values contained within and including 75% to 100%. The same logic applies for all similar expressions such as and not limited to “at least 70%”, “at least 80% identical”, “at least 85% identical”, “at least 95% identical” and the like.

Terms such as “at least 75% identical to 100% identical” also includes all individual values and ranges contained within and including 75% to 100% such as “at least 80% to 100% identical”, at least “85% to 100% identical”, at least 90% to 100% identical”, “at least 95% to 100% identical etc.

It is to be understood herein that the nucleic acid sequences encoding protein(s) or peptide(s) of interest may be codon-optimized. The term “codon-optimized” refers to a sequence for which a codon has been changed for another codon encoding the same amino acid but that is preferred or that performs better in a given organism (increases expression, minimize secondary structures in RNA etc.). “Codon-optimized” sequences may be obtained, using publicly available softwares or via service providers including GenScript (OptimumGene™, U.S. Pat. No. 8,326,547).

As used herein, “pharmaceutical composition” means therapeutically effective amounts of the agent together with pharmaceutically acceptable diluents, preservatives, solubilizers, emulsifiers, adjuvant and/or carriers. A “therapeutically effective amount” as used herein refers to that amount which provides a therapeutic effect for a given condition and administration regimen. Such compositions are liquids or lyophilized or otherwise dried formulations and include diluents of various buffer content (e.g., Tris-HCl., acetate, phosphate), pH and ionic strength, additives such as albumin or gelatin to prevent absorption to surfaces, detergents (e.g., Tween 20, Tween 80, Pluronic F68, bile acid salts). Solubilizing agents (e.g., glycerol, polyethylene glycerol), anti-oxidants (e.g., ascorbic acid, sodium metabisulfite), preservatives (e.g., thimerosal, benzyl alcohol, parabens), etc.

The term “treatment” for purposes of this disclosure refers to both therapeutic treatment and prophylactic or preventative measures, wherein the object is to slow down (lessen) the targeted pathologic condition or disorder. Those in need of treatment include those already with the disorder as well as those prone to have the disorder or those in whom the disorder is to be prevented.

The term “naked vaccine” refers to a non-encapsulated vaccine. The term “naked DNA” or “naked nucleic acids” refers to DNA or nucleic acids that are not associated with protective molecules.

As used herein the term “nanocarrier” includes particles of about 1-1000 nm in size with an interfacial layer that can be composed of different materials. Nanocarriers may be composed of organic materials (e.g., lipids, carbohydrates, proteins etc.), inorganic materials (e.g., graphene oxide, silica, magnetic particles of iron oxide etc.) or of a combination of both (magnetic particles coated with proteins, polymers, polysaccharides etc.).

The term “artificial” with respect to a nucleic acid molecule means that it is not naturally occurring.

The term “naturally occurring” with respect to a sequence means that the sequence is a product of nature.

The term “infectious CCHF virus” as used herein means a CCHF virus that has the potential of infecting humans and/or animals and includes without limitation laboratory isolates and clinical isolates.

The term “immunogenic variant” as used herein refers to a polypeptide variant that is able to elicit an immune response in a human and/or animal.

The term “immunogenic fragment” as used herein refers to a polypeptide fragment that is able to elicit an immune response in a human and/or animal.

The present disclosure provides in one aspect thereof Crimean-Congo Hemorrhagic Fever (CCFH) virus sequences that may be used for vaccination such as DNA vaccination.

The present disclosure provides in a further aspect thereof vectors expressing CCFH virus sequences. The vectors are selected such as to be suitable for DNA vaccination. The vectors of the present disclosure may comprise a transgene encoding a CCHF virus protein and may be used to immunize a host.

In an exemplary embodiment, the vector may encode a CCFH glycoprotein and/or nucleoprotein.

In a further exemplary embodiment, the DNA vector disclosed herein may be able to encode the protein set forth in SEQ ID NO:7, SEQ ID NO: 8 (with or without the ubiquitin portion), SEQ ID NO: 9 (with or without the ubiquitin portion), SEQ ID NO: 10 or SEQ ID NO: 15.

In another exemplary embodiment, the DNA vector disclosed herein may comprise a transgene having the nucleic acid sequence set forth in SEQ ID NO: 3 or a sequence at least 70% identical, at least 75% identical, at least 80% identical, at least 85% identical, at least 90% identical, at least 95% identical or at least 99% identical.

In a further exemplary embodiment, the DNA vector disclosed herein may comprise a transgene having the nucleic acid sequence set forth in SEQ ID NO: 4 or a sequence at least 70% identical, at least 75% identical, at least 80% identical, at least 85% identical, at least 90% identical, at least 95% identical or at least 99% identical.

In another exemplary embodiment, the DNA vector disclosed herein may comprise a transgene having the nucleic acid sequence set forth in SEQ ID NO: 5 or a sequence at least 70% identical, at least 75% identical, at least 80% identical, at least 85% identical, at least 90% identical, at least 95% identical or at least 99% identical.

In another exemplary embodiment, the DNA vector disclosed herein may comprise a transgene having the nucleic acid sequence set forth in SEQ ID NO: 6 or a sequence at least 70% identical, at least 75% identical, at least 80% identical, at least 85% identical, at least 90% identical, at least 95% identical or at least 99% identical.

In a further exemplary embodiment, the DNA vector disclosed herein may comprise a transgene having the nucleic acid sequence set forth in SEQ ID NO: 14 or a sequence at least 70% identical, at least 75% identical, at least 80% identical, at least 85% identical, at least 90% identical, at least 95% identical or at least 99% identical.

In accordance with the present disclosure, the vector portion of the DNA vector may comprise for example, the sequence set forth in SEQ ID NO.1 or a sequence at least 70% identical, at least 75% identical, at least 80%, at least 85%, at least 90%, at least 95% or at least 99% identical to SEQ ID NO:1.

In accordance with the present disclosure, the vector portion of the DNA vector may comprise for example, the sequence set forth in SEQ ID NO.2 or a sequence at least 70% identical, at least 75% identical, at least 80%, at least 85%, at least 90%, at least 95% or at least 99% identical to SEQ ID NO:2.

In accordance with the present disclosure, the vector portion of the DNA vector may comprise for example, the sequence set forth in SEQ ID NO.11 or a sequence at least 70% identical, at least 75% identical, at least 80%, at least 85%, at least 90%, at least 95% or at least 99% identical to SEQ ID NO:11.

In accordance with the present disclosure, the vector portion of the DNA vector may comprise for example, the sequence set forth in SEQ ID NO.12 or a sequence at least 70% identical, at least 75% identical, at least 80%, at least 85%, at least 90%, at least 95% or at least 99% identical to SEQ ID NO:12.

It is to be understood herein that when referring to vectors, the percentage of identity does not take into account the presence of transgene.

The vector may comprise posttranscriptional regulatory elements. In accordance with the present disclosure, the posttranscriptional regulatory element may be from a virus such as for example and without limitation, from Hepatitis B virus or from Woodchuck Hepatitis virus.

In particular embodiment, the sequence of the vector may be as set forth in SEQ ID NO.:1 (pIDV).

In another particular embodiment, the sequence of the vector may be as set forth in SEQ ID NO:11 (pIDV-I).

Yet in a further exemplary embodiment, the sequence of the vector may be as set forth in SEQ ID NO:12 (pIDV-II).

Other vectors suitable for DNA vaccination include for example and without limitations, pVAX (SEQ ID NO:2), pcDNA3.1, gWIZ, NTC9385R and NTC8385 (James A. Williams, Vaccines 2013, 1(3):225-249) etc. As such, the CCHF sequences disclosed herein may be cloned into other DNA vectors.

The present disclosure provides in yet a further aspect thereof DNA vaccines.

In accordance with the present invention, the DNA vaccine may comprise a vector suitable for DNA vaccination and a Crimean-Congo Hemorrhagic Fever virus protein.

In accordance with the present disclosure the DNA vaccine may comprise a pIDV, pIDV-I or pIDV-II vector and a transgene encoding a Crimean-Congo Hemorrhagic Fever virus protein such as for example, a CCFH glycoprotein and/or nucleoprotein.

In accordance with the present disclosure the DNA vaccine may comprise a transgene having the sequence set forth in SEQ ID NO: 3.

Further in accordance with the present disclosure, the DNA vaccine may comprise a transgene having the sequence set forth in SEQ ID NO: 4.

Also in accordance with the present disclosure, the DNA vaccine may comprise a transgene having the sequence set forth in SEQ ID NO: 5.

In accordance with the present disclosure, the DNA vaccine may comprise a transgene having the sequence set forth in SEQ ID NO: 6.

Further in accordance with the present disclosure, the DNA vaccine may comprise a transgene having the sequence set forth in SEQ ID NO: 14.

In a particular embodiment the DNA vaccine may comprise the pIDV-II vector (SEQ ID NO12) and a transgene selected from the group consisting of SEQ ID NO: 3, 4, 5, 6 or 14.

In a particular embodiment, the DNA vaccine may comprise the pIDV-II vector (SEQ ID NO12) or a variant thereof and the nucleic acid sequence set forth in SEQ ID NO:3. In accordance with the present disclosure, the pIDV-II variant may comprise a sequence at least 95% identical or at least 99% identical to SEQ ID NO:12 into which the nucleic acid sequence set forth in SEQ ID NO:3 may be cloned.

In another particular embodiment, the DNA vaccine may comprise the pIDV-II vector (SEQ ID NO12) or a variant thereof and the nucleic acid sequence set forth in SEQ ID NO:14. In accordance with the present disclosure, the pIDV-II variant may comprise a sequence at least 95% identical or at least 99% identical to SEQ ID NO:12 into which the nucleic acid sequence set forth in SEQ ID NO:14 may be cloned.

Exemplary embodiment of DNA vaccine for Crimean-Congo Hemorrhagic Fever virus include for example and without limitation the plasmid set forth in SEQ ID NO:13. Variants having a sequence at least 70% identical, at least 75% identical, at least 80% identical, at least 85% identical, at least 90% identical, at least 95% identical or at least 99% identity with SEQ ID NO:13 are also encompassed.

The present disclosure also provides pharmaceutical compositions comprising the DNA vaccines disclosed herein and a pharmaceutically acceptable carrier.

In accordance with an embodiment of the disclosure, the vaccine may further comprise an adjuvant and/or a plasmid encoding an adjuvanting immunomodulatory molecule such as for example, CpG, CD40L, CD80/86, GM-CSF, ICAM-1, IFN-γ, IL-2, 11-4, IL-7, IL-8, IL-10, IL-12, IL-15, IL-18, MCP-1, M-CSF, MIP-1a, RANTES etc.

The present disclosure also provides for the antigen encoded by any of the transgene disclosed herein. Such antigen may be formulated in pharmaceutical composition for therapeutic use including without limitation for eliciting an immune response and/or for vaccination. Such antigen may also be used as tools in research and development including for example and without limitation in electrophoresis, ELISA assays and the like.

Generally, the specific strain(s), isolate(s) or serotype(s) of pathogen used for generating the vaccine of the present disclosure may be selected from the strain(s), isolate(s) or serotype(s) that is(are) prevalent in a given population. In the case of new outbreaks, the gene expressing the antigen or antigens may be sequenced and cloned into the vector of the present disclosure using methods known in the art involving for example, amplification by polymerase chain reaction, use of restriction enzymes, ligation, transformation of bacteria, sequencing, etc.

The DNA vaccine of the present disclosure may comprise a mixture or combination of the different vectors disclosed herein.

### Variants

Polypeptide variants that are particularly encompassed by the present disclosure include variants of any one of the polypeptide set forth in SEQ ID NO:7, amino acid residues 77 to 720 of SEQ ID NO: 8, amino acid residues 77 to 364 of SEQ ID NO: 9, SEQ ID NO: 10 or SEQ ID NO: 15. Particularly contemplated are polypeptide variants of any one of the polypeptide set forth in SEQ ID NO:7, amino acid residues 77 to 720 of SEQ ID NO: 8, amino acid residues 77 to 364 of SEQ ID NO: 9, SEQ ID NO: 10 or SEQ ID NO: 15 that correspond to a naturally occurring CCHF polypeptide.

Exemplary embodiments of such variants may be found for example in Accession No. DQ019222.1 (representative example of Clade I), DQ211626.1 (representative example of Clade II), AY900141.1 (representative example of Clade III), AB069669.1 (representative example of Clade IV), AY675511.1 (representative example of Clade V), and DQ211628.1 (representative example of Clade VI).

Polypeptide variants of the present disclosure may comprise an insertion, a deletion or an amino acid substitution (conservative or non-conservative) in comparison with an original sequence. These variants may have at least one amino acid residue in its amino acid sequence removed and a different residue inserted in its place.

Conservative substitutions may be made by exchanging an amino acid from one of the groups listed below (group 1 to 6) for another amino acid of the same group.

Other exemplary embodiments of conservative substitutions are shown in Table 1 under the heading of “preferred substitutions”. If such substitutions result in an undesired property, then more substantial changes, denominated “exemplary substitutions” in Table 1, or as further described below in reference to amino acid classes, may be introduced and the products screened.

It is known in the art that variants may be generated by substitutional mutagenesis and retain the biological activity of the polypeptides of the present disclosure. These variants have at least one amino acid residue in the amino acid sequence removed and a different residue inserted in its place. Examples of substitutions identified as “conservative substitutions” are shown in Table 1. If such substitutions result in a change not desired, then other type of substitutions, denominated “exemplary substitutions” in Table 1A, or as further described herein in reference to amino acid classes, are introduced and the products screened.

Naturally occurring residues are divided into groups based on common side chain properties:
(group 1) hydrophobic: norleucine, methionine (Met), Alanine (Ala), Valine (Val), Leucine (Leu), Isoleucine (Ile)
(group 2) neutral hydrophilic: Cysteine (Cys), Serine (Ser), Threonine (Thr), Asparagine (Asn), Glutamine (Gln),
(group 3) acidic: Aspartic acid (Asp), Glutamic acid (Glu)
(group 4) basic: Histidine (His), Lysine (Lys), Arginine (Arg)
(group 5) residues that influence chain orientation: Glycine (Gly), Proline (Pro); and
(group 6) aromatic: Tryptophan (Trp), Tyrosine (Tyr), Phenylalanine (Phe)

Non-conservative substitutions will entail exchanging a member of one of these classes for another.

TABLE 1
Exemplary amino acid substitutions

| Original | Exemplary | Conservative |
|---|---|---|
| residue | substitution | substitution |
| Ala (A) | Val, Leu, Ile | Val |
| Arg (R) | Lys, Gln, Asn | Lys |
| Asn (N) | Gln, His, Lys, Arg, Asp | Gln |
| Asp (D) | Glu, Asn | Glu |
| Cys (C) | Ser, Ala | Ser |
| Gln (Q) | Asn; Glu | Asn |
| Glu (E) | Asp, Gln | Asp |
| Gly (G) | Ala | Ala |
| His (H) | Asn, Gln, Lys, Arg, | Arg |
| Ile (I) | Leu, Val, Met, Ala, Phe, | Leu |
| | norleucine | |
| Leu (L) | Norleucine, Ile, Val, Met, | Ile |
| | Ala, Phe | |
| Lys (K) | Arg, Gln, Asn | Arg |
| Met (M) | Leu, Phe, Ile | Leu |
| Phe (F) | Leu, Val, Ile, Ala, Tyr | Tyr |
| Pro (P) | Ala | Ala |
| Ser (S) | Thr | Thr |
| Thr (T) | Ser | Ser |
| Trp (W) | Tyr, Phe | Tyr |
| Tyr (Y) | Trp, Phe, Thr, Ser | Phe |
| Val (V) | Ile, Leu, Met, Phe, Ala, | Leu |
| | norleucine | |

Generally, the degree of similarity and identity between variable chains is determined herein using the Blast2 sequence program (Tatiana A. Tatusova, Thomas L. Madden (1999), “Blast 2 sequences—a new tool for comparing protein and nucleotide sequences”, FEMS Microbiol Lett. 174:247-250) using default settings, i.e., blastp program, BLOSUM62 matrix (open gap 11 and extension gap penalty 1; gapx dropoff 50, expect 10.0, word size 3) and activated filters.

Percent identity will therefore be indicative of amino acids which are identical in comparison with the original peptide and which may occupy the same or similar position.

Percent similarity will be indicative of amino acids which are identical and those which are replaced with conservative amino acid substitution in comparison with the original peptide at the same or similar position.

Variants of the present disclosure therefore comprise those which may have at least 70%, 75%, 80%, 81%, 82%, 83%, 84%, 85%, 86%, 87%, 88%, 89%, 90%, 91%, 92%, 93%, 94%, 95%, 96%, 97%, 98%, 99% or 100% sequence identity with an original sequence or a portion of an original sequence.

Those skilled in the art will also recognize that short oligonucleotides sequences may be prepared based on the nucleic acid sequences described herein. For example, oligonucleotides having 10 to 20 nucleotides or more may be prepared for specific hybridization or for use in amplification of nucleic acid sequences. As such, the present disclosure also relates to complements of the nucleic acid molecules described herein. The complements may comprise a sequence of at least from 10 to 20 nucleotides that is complementary to that of the nucleic acid molecules of the present disclosure. The complements may also be longer so as to be complementary to the full sequence. In some instance the complement may comprise a sequence that is complementary to that of the nucleic acid molecules and other unrelated sequences.

### Method of Manufacturing

Methods for manufacturing DNA vectors for vaccination are known in the art and are based on guidance from the FDA (USA Food and Drug Administration. Guidance for Industry: Considerations for Plasmid DNA Vaccines for Infectious Disease Indications. Rockville, Md., USA: 2007) or the EMA (European Medicines Agency. Note for Guidance on the Quality, Preclinical and Clinical Aspects of Gene Transfer Medicinal Products. London, UK: 2001. CPMP/BWP/3088/99; Presence of the Antibiotic Resistance Marker Gene nptII in GM Plants and Food and Feed Uses. London, UK: 2007. EMEA/CVMP/56937/2007).

Exemplary methods of manufacturing are reviewed in Williams J. A., 2013 (Vaccines, 1(3): 225-249, 2013). Processes for high-scale production and purification are also disclosed in Carnes, A. E. and J. A. Williams, 2007 (Recent Patents on Biotechnology, 1:151-66, 2007).

Plasmid DNA production is typically performed in endA (DNA-specific endonuclease I), recA (DNA recombination) deficient E. coli K12 strains such as DH5α, DH5, DH1, XL1Blue, GT115, JM108, DH10B, or endA, recA engineered derivatives of alternative strains such as MG1655, or BL21.

Transformed bacteria are fermented using for example, fed-batch fermentation processes. Clinical grade DNA vector can be obtained by various methods (e.g., HyperGRO™) through service providers such as Aldevron, Eurogentec and VGXI.

DNA vectors are then purified to remove bacterial debris and impurities (RNA, genomic DNA, endotoxins) and formulated with a suitable carrier (for research purposes) or pharmaceutical carrier (for pre-clinical or clinical applications).

### Pharmaceutical Compositions

DNA vectors of the present disclosure may be administered as a pharmaceutical composition, which may comprise for example, the DNA vector(s) and a pharmaceutically acceptable carrier.

The pharmaceutical composition may comprise a single DNA vector species encoding one or more antigens. The one or more antigens may be, for example, from the same pathogen, from closely-related pathogens, or from different pathogens.

Alternatively, the pharmaceutical composition may comprise a mixture of DNA vector species (multiple DNA vector species) each encoding different antigens. For example, the different antigens may be from the same pathogen, from closely-related pathogens, or from different pathogens.

The pharmaceutical composition may further comprise additional elements for increasing uptake of the DNA vector by the cells, its transport in the nucleic, expression of the transgene, secretion, immune response, etc.

The pharmaceutical composition may comprise for example, adjuvant molecule(s). The adjuvant molecule(s) may be encoded by the DNA vector that encodes the antigen or by another DNA vector. Encoded adjuvant molecule(s) may include DNA- or RNA-based adjuvant (CpG oligonucleotides, immunostimulatory RNA, etc.) or protein-based immunomodulators.

The adjuvant molecule(s) may be co-administered with the DNA vectors.

Adjuvants include, but are not limited to, mineral salts (e.g., AlK(SO₄)2, AlNa(SO₄)2, AlNH(SO₄)₂, silica, alum, Al(OH)₃, Ca₃(PO₄)₂, kaolin, or carbon), polynucleotides with or without immune stimulating complexes (ISCOMs), CpG oligonucleotides, immunostimulatory RNA, poly IC or poly AU acids, saponins such as QS21, QS17, and QS7 (U.S. Pat. Nos. 5,057,540; 5,650,398; 6,524,584; 6,645,495), monophosphoryl lipid A, such as 3-de-O-acylated monophosphoryl lipid A (3D-MPL), imiquimod, lipid-polymer matrix (ENABL™ adjuvant), Emulsigen-D™ etc.

A pIDV, pIDV-I or vector encoding the antigen disclosed herein may be formulated for administration by injection (e.g., intramuscular, intradermal, transdermal, subcutaneously) or for mucosal administration (oral, intranasal).

In accordance with the present disclosure, the pharmaceutical composition may be formulated into nanoparticles.

### Method of Administration

The DNA vectors and DNA vaccines of the present disclosure may be administered to humans or to animals (non-human primates, cattle, rabbits, mice, rats, sheep, goats, horses, birds, poultry, fish, etc.). The DNA vector may thus be used as a vaccine in order to trigger an immune response against an antigen of interest in a human or animal.

Advantageously, the DNA vectors and DNA vaccines of the present disclosure generate an immune response even when administered as a naked vaccine.

The pIDV, pIDV-I or vector encoding the antigen disclosed herein may be administered alone (e.g., as a single dose or in multiple doses) or co-administered with a recombinant antigen, with a viral vaccine (live (e.g., replication competent or not), attenuated, inactivated, etc.), with suitable therapy for modulating or boosting the host's immune response such as for example, adjuvants, immunomodulators (cytokine, chemokines, checkpoint inhibitors, etc.), etc. A pIDV, pIDV-I or pIDV-II vector encoding the antigen disclosed herein may also be co-administered with a plasmid encoding molecules that may act as adjuvant. In accordance with the present disclosure, such adjuvant molecules may also be encoded by the pIDV, pIDV-I or pIDV-II vector (e.g., CpG motifs, cytokine, chemokines, etc.).

In some instances, the pIDV, pIDV-I or pIDV-II vector encoding the antigen disclosed herein may be administered first (for priming) and the recombinant antigen or viral vaccine may be administered subsequently (as a boost), or vice versa.

The pIDV, pIDV-I or pIDV-II vector encoding the antigen disclosed herein may be administered by injection intramuscularly, intradermally, transdermally, subcutaneously, to the mucosa (oral, intranasal), etc.

In accordance with the present disclosure, the vaccine may be administered by a physical delivery system including via electroporation, a needleless pressure-based delivery system, particle bombardment, etc.

Following administration, the host's immune response towards the antigen may be assessed using methods known. In some instances, the level of antibodies against the antigen may be measured by ELISA assay or by other methods known by a person skilled in the art. The cellular immune response towards the antigen may be assessed by ELISPOT or by other methods known by a person skilled in the art.

In the case of pre-clinical studies in animals, the level of protection against the pathogen may be determined by challenge experiments where the pathogen is administered to the animal and the animal's health or survival is assessed. The level of protection conferred by the vaccine expressing a tumor antigen may be determined by tumor shrinkage or inhibition of tumor growth in animal models carrying the tumor.

Protective efficacy of the DNA vaccines of the present disclosure may be determined in lethal animal models such as for example the STAT-1 knockout mouse model (C57BL6 background) and in interferon α/β (IFN-α/β) receptor 1 knockout (IFNAR^{-/-}) mouse models (C57BL/6 or A129 background) disclosed in Bente D A et al., (J. Virol. 2010; 84(21):11089-11100), Zivcec M et al., (The Journal of infectious diseases. 2013; 207(12):1909-1921) and Bereczky S et al. (J Gen Virol. 2010; 91(Pt6):1473-1477) the entire content of which is incorporated herein by reference. Animals may be thus be administered with the DNA vaccines of the present disclosure and subsequently challenged with CCHF Turkey strains (e.g., isolate 812955).

In addition to the embodiments described and provided in this disclosure, the following non-limiting embodiments are particularly contemplated.
1. A nucleic acid molecule having the sequence set forth in SEQ ID NO:3.
2. A nucleic acid molecule having the sequence set forth in having a sequence at least 70% identical, at least 75% identical, at least 80% identical, at least 85% identical, at least 90% identical, at least 95% identical or at least 99% identical to SEQ ID NO:3.
3. A nucleic acid molecule comprising a fragment of SEQ ID NO:3.
4. A nucleic acid molecule comprising a fragment of a nucleic acid molecule having a sequence at least 70% identical, at least 75% identical, at least 80% identical, at least 85% identical, at least 90% identical, at least 95% identical or at least 99% identical to SEQ ID NO:3.
5. A nucleic acid molecule having the sequence set forth in SEQ ID NO:4.
6. A nucleic acid molecule having the sequence set forth in nucleotides 229-2163 of SEQ ID NO:4.
7. A nucleic acid molecule having a sequence at least 70% identical, at least 75% identical, at least 80% identical, at least 85% identical, at least 90% identical, at least 95% identical or at least 99% identical to SEQ ID NO:4.
8. A nucleic acid molecule having a sequence at least 70% identical, at least 75% identical, at least 80% identical, at least 85% identical, at least 90% identical, at least 95% identical or at least 99% identical to the sequence set forth in nucleotides 229-2163 of SEQ ID NO:4.
9. A nucleic acid molecule comprising a fragment of SEQ ID NO:4.
10. A nucleic acid molecule comprising a fragment of the sequence set forth in nucleotides 229-2163 of SEQ ID NO:4.
11. A nucleic acid molecule comprising a fragment of a nucleic acid molecule having a sequence at least 70% identical, at least 75% identical, at least 80% identical, at least 85% identical, at least 90% identical, at least 95% identical or at least 99% identical to SEQ ID NO:4.
12. A nucleic acid molecule comprising a fragment of a nucleic acid molecule having a sequence at least 70% identical, at least 75% identical, at least 80% identical, at least 85% identical, at least 90% identical, at least 95% identical or at least 99% identical to the sequence set forth in nucleotides 229-2163 of SEQ ID NO:4
13. A nucleic acid molecule having the sequence set forth in SEQ ID NO:5.
14. A nucleic acid molecule having the sequence set forth in nucleotides 229-1092 of SEQ ID NO:5.
15. A nucleic acid molecule having a sequence at least 70% identical, at least 75% identical, at least 80% identical, at least 85% identical, at least 90% identical, at least 95% identical or at least 99% identical to SEQ ID NO:5.
16. A nucleic acid molecule having a sequence at least 70% identical, at least 75% identical, at least 80% identical, at least 85% identical, at least 90% identical, at least 95% identical or at least 99% identical to the sequence set forth in nucleotides 229-1092 of SEQ ID NO:5.
17. A nucleic acid molecule comprising a fragment of SEQ ID NO:5.
18. A nucleic acid molecule comprising a fragment of the sequence set forth in nucleotides 229-1092 of SEQ ID NO:5.
19. A nucleic acid molecule comprising a fragment of a nucleic acid molecule having a sequence at least 70% identical, at least 75% identical, at least 80% identical, at least 85% identical, at least 90% identical, at least 95% identical or at least 99% identical to SEQ ID NO:5.
20. A nucleic acid molecule comprising a fragment of a nucleic acid molecule having a sequence at least 70% identical, at least 75% identical, at least 80% identical, at least 85% identical, at least 90% identical, at least 95% identical or at least 99% identical to the sequence set forth in nucleotides 229-1092 of SEQ ID NO:5.
21. A nucleic acid molecule having the sequence set forth in SEQ ID NO:6.
22. A nucleic acid molecule having a sequence at least 70% identical, at least 75% identical, at least 80% identical, at least 85% identical, at least 90% identical, at least 95% identical or at least 99% identical to SEQ ID NO:6.
23. A nucleic acid molecule comprising a fragment of SEQ ID NO:6.
24. A nucleic acid molecule comprising a fragment of a nucleic acid molecule having a sequence at least 70% identical, at least 75% identical, at least 80% identical, at least 85% identical, at least 90% identical, at least 95% identical or at least 99% identical to SEQ ID NO:6.
25. A nucleic acid molecule having the sequence set forth in SEQ ID NO:14.
26. A nucleic acid molecule having a sequence at least 70% identical, at least 75% identical, at least 80% identical, at least 85% identical, at least 90% identical, at least 95% identical or at least 99% identical to SEQ ID NO:14.
27. A nucleic acid molecule comprising a fragment of SEQ ID NO:14.
28. A nucleic acid molecule comprising a fragment of a nucleic acid molecule having a sequence at least 80% identical, at least 85% identical, at least 90% identical, at least 95% identical or at least 99% identical to SEQ ID NO:14.
29. The nucleic acid molecule of any one of the preceding embodiments, wherein the nucleic acid encodes a CCHF polypeptide of an infectious CCHF virus.
30. The nucleic acid molecule of any one of the preceding embodiments, wherein the nucleic acid encodes a CCHF polypeptide variant.
31. The nucleic acid molecule of any one of the preceding embodiments, wherein the nucleic acid encodes a CCHF polypeptide fragment.
32. The nucleic acid molecule of any one of the preceding embodiments, wherein the nucleic acid encodes an antigen from a CCHF Turkey strain.
33. The nucleic acid molecule of any one of the preceding embodiments, wherein the nucleic acid encodes an antigen of the Kelkit (kk) Turkey 06 strain.
34. The nucleic acid molecule of any one of the preceding embodiments, wherein the nucleic acid encodes the M segment of Turkey isolate 812955 (Accession number KY362519.1) or a sequence at least 85% identical, at least 90% identical, at least 95% identical, or at least 99% identical.
35. The nucleic acid molecule of any one of the preceding embodiments, wherein the nucleic acid encodes the polypeptide set forth in amino acid residues 77 to 720 of SEQ ID NO: 8 or a polypeptide fragment thereof.
36. The nucleic acid molecule of any one of the preceding embodiments, wherein the nucleic acid encodes the polypeptide set forth in SEQ ID NO:8 or a polypeptide fragment thereof.
37. The nucleic acid molecule of any one of the preceding embodiments, wherein the nucleic acid encodes the polypeptide set forth in amino acid residues 77 to 364 of SEQ ID NO: 9 or a polypeptide fragment thereof.
38. The nucleic acid molecule of any one of the preceding embodiments, wherein the nucleic acid encodes the polypeptide set forth in SEQ ID NO:9 or a polypeptide fragment thereof.
39. The nucleic acid molecule of any one of the preceding embodiments, wherein the nucleic acid encodes the polypeptide set forth in SEQ ID NO:10 or a polypeptide fragment thereof
40. The nucleic acid molecule of any one of the preceding embodiments, wherein the nucleic acid encodes the polypeptide set forth in SEQ ID NO: 15 or a polypeptide fragment thereof
41. The nucleic acid molecule of any one of the preceding embodiments, wherein the nucleic acid encodes a polypeptide variant having at least 70% identity with the polypeptide set forth in SEQ ID NO:7 or a fragment thereof.
42. The nucleic acid molecule of any one of the preceding embodiments, wherein the nucleic acid encodes a polypeptide variant having at least 75% identity with the polypeptide set forth in SEQ ID NO: 7 or a fragment thereof.
43. The nucleic acid molecule of any one of the preceding embodiments, wherein the nucleic acid encodes a polypeptide variant having at least 80% identity with the polypeptide set forth in SEQ ID NO: 7 or a fragment thereof.
44. The nucleic acid molecule of any one of the preceding embodiments, wherein the nucleic acid encodes a polypeptide variant having at least 85% identity with the polypeptide set forth in SEQ ID NO: 7 or a fragment thereof.
45. The nucleic acid molecule of any one of the preceding embodiments, wherein the nucleic acid encodes a polypeptide variant having at least 90% identity with the polypeptide set forth in SEQ ID NO: 7 or a fragment thereof.
46. The nucleic acid molecule of any one of the preceding embodiments, wherein the nucleic acid encodes a polypeptide variant having at least 95% identity with the polypeptide set forth in SEQ ID NO: 7 or a fragment thereof.
47. The nucleic acid molecule of any one of the preceding embodiments, wherein the nucleic acid encodes a polypeptide variant having at least 99% identity with the polypeptide set forth in SEQ ID NO: 7 or a fragment thereof.
48. The nucleic acid molecule of any one of the preceding embodiments, wherein the nucleic acid encodes a polypeptide variant having at least 70% identity, at least 75% identity at least 80% identity, at least 85% identity, at least 90% identity, at least 95% identity or at least 99% identity with the polypeptide set forth in amino acid residues 77 to 720 of SEQ ID NO: 8 or a fragment thereof
49. The nucleic acid molecule of any one of the preceding embodiments, wherein the nucleic acid encodes a polypeptide variant having at least 70% identity, at least 75% identity at least 80% identity, at least 85% identity, at least 90% identity, at least 95% identity or at least 99% identity with the polypeptide set forth in SEQ ID NO: 8 or a fragment thereof.
50. The nucleic acid molecule of any one of the preceding embodiments, wherein the nucleic acid encodes a polypeptide variant having at least 70% identity, at least 75% identity at least 80% identity, at least 85% identity, at least 90% identity, at least 95% identity or at least 99% identity with the polypeptide set forth in amino acid residues 77 to 364 of SEQ ID NO: 9 or a fragment thereof
51. The nucleic acid molecule of any one of the preceding embodiments, wherein the nucleic acid encodes a polypeptide variant having at least 70% identity, at least 75% identity at least 80% identity, at least 85% identity, at least 90% identity, at least 95% identity or at least 99% identity with the polypeptide set forth in SEQ ID NO: 9 or a fragment thereof.
52. The nucleic acid molecule of any one of the preceding embodiments, wherein the nucleic acid encodes a polypeptide variant having at least 70% identity, at least 75% identity at least 80% identity, at least 85% identity, at least 90% identity, at least 95% identity or at least 99% identity with the polypeptide set forth in SEQ ID NO:10 or a fragment thereof.
53. The nucleic acid molecule of any one of the preceding embodiments, wherein the nucleic acid encodes a polypeptide variant having at least 70% identity with the polypeptide set forth in SEQ ID NO: 15 or a fragment thereof.
54. The nucleic acid molecule of any one of the preceding embodiments, wherein the nucleic acid encodes a polypeptide variant having at least 75% identity with the polypeptide set forth in SEQ ID NO: 15 or a fragment thereof.
55. The nucleic acid molecule of any one of the preceding embodiments, wherein the nucleic acid encodes a polypeptide variant having at least 80% identity with the polypeptide set forth in SEQ ID NO: 15 or a fragment thereof.
56. The nucleic acid molecule of any one of the preceding embodiments, wherein the nucleic acid encodes a polypeptide variant having at least 85% identity with the polypeptide set forth in SEQ ID NO: 15 or a fragment thereof.
57. The nucleic acid molecule of any one of the preceding embodiments, wherein the nucleic acid encodes a polypeptide variant having at least 90% identity with the polypeptide set forth in SEQ ID NO: 15 or a fragment thereof.
58. The nucleic acid molecule of any one of the preceding embodiments, wherein the nucleic acid encodes a polypeptide variant having at least 95% identity with the polypeptide set forth in SEQ ID NO: 15 or a fragment thereof.
59. The nucleic acid molecule of any one of the preceding embodiments, wherein the nucleic acid encodes a polypeptide variant having at least 99% identity with the polypeptide set forth in SEQ ID NO: 15 or a fragment thereof.
60. The nucleic acid molecule of any one of the preceding embodiments, wherein the CCHF polypeptide fragment comprises at least 10 amino acid residues.
61. The nucleic acid molecule of any one of the preceding embodiments, wherein the CCHF polypeptide fragment comprises at least 20 amino acid residues.
62. The nucleic acid molecule of any one of the preceding embodiments, wherein the CCHF polypeptide fragment comprises at least 30 amino acid residues.
63. The nucleic acid molecule of any one of the preceding embodiments, wherein the CCHF polypeptide fragment comprises at least 40 amino acid residues.
64. The nucleic acid molecule of any one of the preceding embodiments, wherein the CCHF polypeptide fragment comprises at least 50 amino acid residues.
65. The nucleic acid molecule of any one of the preceding embodiments, wherein the CCHF polypeptide fragment comprises at least 100 amino acid residues.
66. The nucleic acid molecule of any one of the preceding embodiments, wherein the CCHF polypeptide fragment comprises at least 500 amino acid residues.
67. The nucleic acid molecule of any one of the preceding embodiments, wherein the CCHF polypeptide fragment comprises at least 1000 amino acid residues.
68. The nucleic acid molecule of any one of the preceding embodiments, wherein the CCHF polypeptide fragment comprises at least 1500 amino acid residues.
69. The nucleic acid molecule of any one of the preceding embodiments, wherein the nucleic acid encodes the polypeptide set forth in SEQ ID NO:7 or a polypeptide fragment thereof.
70. The nucleic acid molecule of any one of the preceding embodiments, wherein the nucleic acid comprises DNA.
71. The nucleic acid molecule of any one of the preceding embodiments, wherein the nucleic acid comprises RNA.
72. An expression cassette comprising the nucleic acid molecule sequence of any one of the preceding embodiments and regulatory elements.
73. A vector comprising the nucleic acid sequence of any one of the preceding embodiments or the expression cassette of any one of the preceding embodiments.
74. The vector any one of the preceding embodiments, wherein the vector is a vector for DNA vaccination. 75. The vector of any one of the preceding embodiments, wherein the vector is pVAX.
76. The vector of any one of the preceding embodiments, wherein the vector is pIDV (SEQ ID NO:1).
77. The vector of any one of the preceding embodiments, wherein the vector is pIDV-I (SEQ ID NO:11).
78. The vector of any one of the preceding embodiments, wherein the vector is pIDV-II (SEQ ID NO:12).
79. A set of vectors comprising at least one vector comprising the nucleic acid sequence of any one of the preceding embodiments or the expression cassette of any one of the preceding embodiments and at least one vector comprising a nucleic acid sequence encoding another antigen.
80. The set of vectors of any one of the preceding embodiments, wherein at least one vector comprises a nucleic acid sequence encoding a CCHF glycoprotein and at least one vector comprises a nucleic acid sequence encoding the other antigen.
81. The set of vectors of any one of the preceding embodiments, wherein the other antigen is another CCHF antigen.
82. The vector of any one of the preceding embodiments, wherein the vector is a viral vector.
83. The vector of any one of the preceding embodiments, wherein the viral vector is from a DNA virus.
84. A vector comprising the nucleic acid sequence set forth in SEQ ID NO:13 or a sequence at least 70% identical, at least 75% identical, at least 80% identical, at least 85% identical, at least 90% identical, at least 95% identical or at least 99% identical.
85. The vector of any one of the preceding embodiments, wherein the vector portion of the vector comprises a sequence at least 70% identical, at least 75% identical, at least 80% identical, at least 85% identical, at least 90% identical, at least 95% identical or at least 99% identical to SEQ ID NO:12.
86. The nucleic acid molecule, vector or set of vectors of any one of the preceding embodiments, wherein the nucleic acid or vector is encapsulated into a nanocarrier.
87. A pharmaceutical composition comprising the nucleic acid molecule, vector or set of vectors of any one of the preceding embodiments and a pharmaceutically acceptable carrier.
88. The pharmaceutical composition of any of the preceding embodiments, wherein the nucleic acid molecule, vector or set of vectors is encapsulated into a nanocarrier.
89. A method of immunizing a host, the method comprising administering the pharmaceutical composition of any one of the preceding embodiments to the host.
90. The method of any one of the preceding embodiments, wherein the host is a human.
91. The method of any one of the preceding embodiments, wherein the host is an animal.
92. The method of any one of the preceding embodiments, wherein the pharmaceutical composition is administered by injection.
93. The method of any one of the preceding embodiments, wherein the pharmaceutical composition is administered by electroporation.
94. The method of any one of the preceding embodiments, wherein the pharmaceutical composition is administered intradermally, transdermally or intramuscularly.
95. The method of any one of the preceding embodiments, wherein the pharmaceutical composition is administered at a mucosal site.
96. A method of manufacturing the vector of any one of the preceding embodiments, the method comprising fermenting microorganisms transformed with the vector.
97. The method of manufacturing of any one of the preceding embodiments, further comprising isolating substantially purified vectors from the microorganism.
98. A codon-optimized nucleic acid comprising a sequence encoding a Crimean-Congo Hemorrhagic Fever (CCHF) Virus antigen or encoding a fragment thereof, wherein the codon-optimized nucleic acid sequence or fragment thereof comprises SEQ ID NO.: 3, a fragment thereof or a complement thereof.
99. A codon-optimized nucleic acid comprising a sequence encoding a Crimean-Congo Hemorrhagic Fever (CCHF) Virus antigen or encoding a fragment thereof, wherein the codon-optimized nucleic acid sequence or fragment thereof comprises nucleotides 229-2163 of SEQ ID NO.:4, a fragment thereof or a complement thereof.
100. A codon-optimized nucleic acid comprising a sequence encoding a Crimean-Congo Hemorrhagic Fever (CCHF) Virus antigen or encoding a fragment thereof, wherein the codon-optimized nucleic acid sequence or fragment thereof comprises nucleotides 229-1092 of SEQ ID NO.:5, a fragment thereof or a complement thereof.
101. A codon-optimized nucleic acid comprising a sequence encoding a Crimean-Congo Hemorrhagic Fever (CCHF) Virus antigen or encoding a fragment thereof, wherein the codon-optimized nucleic acid sequence or fragment thereof comprises SEQ ID NO:14, a fragment thereof or a complement thereof.
102. A DNA vector comprising the nucleic acid sequence of any one of the preceding embodiments.
103. A DNA vector comprising a nucleic acid sequence encoding a CCHF virus antigen and a vector's backbone having a sequence at least 75%, at least 80%, at least 85%, at least 90%, at least 95%, at least 99% identical or identical to SEQ ID NO.:12.
104. A DNA vector comprising a nucleic acid sequence encoding a CCHF glycoprotein or a fragment thereof, wherein the nucleic acid sequence comprises SEQ ID NO.: 3 or a fragment thereof
105. A DNA vector comprising a nucleic acid sequence encoding a CCHF glycoprotein or a fragment thereof, wherein the nucleic acid sequence comprises nucleotides 229-2163 of SEQ ID NO.:4 or a fragment thereof.
106. A DNA vector comprising a nucleic acid sequence encoding a CCHF glycoprotein or a fragment thereof, wherein the nucleic acid sequence comprises SEQ ID NO.:4 or a fragment thereof
107. A DNA vector comprising a nucleic acid sequence encoding a CCHF glycoprotein or a fragment thereof, wherein the nucleic acid sequence comprises nucleotides 229-1092 of SEQ ID NO.:5 or a fragment thereof.
108. A DNA vector comprising a nucleic acid sequence encoding a CCHF glycoprotein or a fragment thereof, wherein the nucleic acid sequence comprises SEQ ID NO.:5 or a fragment thereof
109. A DNA vector comprising a nucleic acid sequence encoding a CCHF nucleoprotein or a fragment thereof, wherein the nucleic acid sequence comprises SEQ ID NO.:6 or a fragment thereof
110. A DNA vector comprising a nucleic acid sequence encoding a CCHF glycoprotein or a fragment thereof, wherein the nucleic acid sequence comprises SEQ ID NO.:14 or a fragment thereof
111. The DNA vector of any one of the preceding embodiments, wherein the vector's backbone comprises a nucleic acid having a sequence at least 75% identical to 100% identical to SEQ ID NO.:1.
112. The DNA vector of any one of the preceding embodiments, wherein the vector's backbone comprises a nucleic acid having a sequence at least 75% identical to 100% identical to SEQ ID NO.:2.
113. The DNA vector of any one of the preceding embodiments, wherein the vector's backbone comprises a nucleic acid having a sequence at least 75% identical to 100% identical to SEQ ID NO.:11.
114. The DNA vector of any one of the preceding embodiments, wherein the vector's backbone comprises a nucleic acid having a sequence at least 75% identical to 100% identical to SEQ ID NO.:12.
115. A DNA vaccine comprising the nucleic acid molecule or DNA vector of any one of the preceding embodiments or a combination thereof.
116. A DNA vaccine comprising a nucleic acid sequence encoding a CCHF virus antigen expressed by the DNA vector set forth in SEQ ID NO:12.
117. The DNA vector or the DNA vaccine of any one of the preceding embodiments, wherein the CCHF virus antigen comprises the M segment of CCHF.
118. The DNA vector or DNA vaccine of any one of the preceding embodiments, wherein the CCHF virus antigen comprises the Gn and/or Gc segment of CCHF.
119. The DNA vector or DNA vaccine of any one of the preceding embodiments, wherein the CCHF virus antigen is from the Kelkit (kk) Turkey 06 strain.
120. The DNA vector or DNA vaccine of any one of the preceding embodiments, wherein the CCHF virus antigen comprises the M segment of Turkey isolate 812955 (Accession number KY362519.1) or a sequence at least 85% identical, at least 90% identical, at least 95% identical, or at least 99% identical.
121. The DNA vector or DNA vaccine of any one of the preceding embodiments, wherein the nucleic acid encodes a CCHF virus antigen having at least 85% identity with the polypeptide set forth in SEQ ID NO: 15 or a fragment thereof.
122. The DNA vector or DNA vaccine of any one of the preceding embodiments, wherein the nucleic acid encodes a CCHF virus antigen having at least 90% identity with the polypeptide set forth in SEQ ID NO: 15 or a fragment thereof.
123. The DNA vector or DNA vaccine of any one of the preceding embodiments, wherein the nucleic acid encodes a CCHF virus antigen having at least 95% identity with the polypeptide set forth in SEQ ID NO: 15 or a fragment thereof.
124. The DNA vector or DNA vaccine of any one of the preceding embodiments, wherein the nucleic acid encodes a CCHF virus antigen having at least 99% identity with the polypeptide set forth in SEQ ID NO: 15 or a fragment thereof.
125. The DNA vector or DNA vaccine of any one of the preceding embodiments, wherein the nucleic acid encodes a CCHF virus antigen is identical to SEQ ID NO:15.
126. The DNA vector or DNA vaccine of any one of the preceding embodiments, wherein the nucleic acid encodes a CCHF virus antigen having at least 85% identity with the polypeptide set forth in SEQ ID NO: 7 or a fragment thereof.
127. The DNA vector or DNA vaccine of any one of the preceding embodiments, wherein the nucleic acid encodes a CCHF virus antigen having at least 90% identity with the polypeptide set forth in SEQ ID NO: 7 or a fragment thereof.
128. The DNA vector or DNA vaccine of any one of the preceding embodiments, wherein the nucleic acid encodes a CCHF virus antigen having at least 95% identity with the polypeptide set forth in SEQ ID NO: 7 or a fragment thereof.
129. The DNA vector or DNA vaccine of any one of the preceding embodiments, wherein the nucleic acid encodes a CCHF virus antigen having at least 99% identity with the polypeptide set forth in SEQ ID NO: 7 or a fragment thereof.
130. The DNA vector or DNA vaccine of any one of the preceding embodiments, wherein the nucleic acid encodes a CCHF virus antigen is identical to SEQ ID NO:7.
131. A DNA vaccine comprising the nucleic acid sequence set forth in SEQ ID NO:3.
132. A DNA vaccine comprising the nucleic acid sequence set forth in SEQ ID NO:4 or the nucleic acid sequence set forth in nucleotides 229-2163 of SEQ ID NO:4.
133. A DNA vaccine comprising the nucleic acid sequence set forth in SEQ ID NO:4 or the nucleic acid sequence set forth in SEQ ID NO:4.
134. A DNA vaccine comprising the nucleic acid sequence set forth in SEQ ID NO:5 or the nucleic acid sequence set forth in nucleotides 229-1092 of SEQ ID NO:5.
135. A DNA vaccine comprising the nucleic acid sequence set forth in SEQ ID NO:5 or the nucleic acid sequence set forth in SEQ ID NO:5.
136. A DNA vaccine comprising the nucleic acid sequence set forth in SEQ ID NO: 6.
137. A DNA vaccine comprising the nucleic acid sequence set forth in SEQ ID NO:14.
138. A DNA vaccine comprising the nucleic acid sequence set forth in SEQ ID NO:3 and a vector comprising a sequence at least 70% identical, at least 75% identical, at least 80% identical, at least 85% identical, at least 90% identical, at least 95% identical or at least 99% identical to SEQ ID NO: 12.
139. A DNA vaccine comprising the nucleic acid sequence set forth in SEQ ID NO:14 and a vector comprising a sequence at least 70% identical, at least 75% identical, at least 80% identical, at least 85% identical, at least 90% identical, at least 95% identical or at least 99% identical to SEQ ID NO: 12.
140. The DNA vaccine of any one of the preceding embodiments, wherein the vector has a sequence at least 70% identical, at least 75% identical, at least 80% identical to SEQ ID NO:12.
141. The DNA vaccine of any one of the preceding embodiments, wherein the vector has a sequence at least 90% identical to SEQ ID NO:12.
142. The DNA vaccine of any one of the preceding embodiments, wherein the vector has a sequence at least 99% identical to SEQ ID NO:12.
143. The vector or DNA vaccine of any one of the preceding embodiments, wherein the vector is able to express a transgene.
144. A DNA vaccine comprising the nucleic acid sequence set forth in SEQ ID NO:13.
145. A method of immunizing a host comprising administering the nucleic acid, vector, vaccine, composition or pharmaceutical composition of any of the preceding embodiments.

All patents, patent applications, and publications referred to herein are incorporated by reference in their entirety.

### Example 1

The pIDV-I plasmid was initially designed in silico based on insertion of 2919 bp fragment that includes CMV enhancer, cloning Chicken β-actin/Rabit β-globin hybrid promoter, site KpnI and BglII, β-globin polyadenylation signal and 3′ flanking region of rabbit β-Globin from recombinant plasmid pCAGS at the sites of SpeI and HindIII, into pVAX1 plasmid which was in silico linearized with NruI and HindIII restriction enzymes by Genius software. Thus, nucleotide 32-1054 which contains the CMV promoter, the T7 promoter, the multiple cloning sites and the bGH PA terminator were removed from pVAX1. Circularized plasmid was synthesized (GenScript).

The vector has been designed to allow easy insertion and subsequent high expression of exogenous genes in a wide variety of mammalian cells. The vectors share a common structure of a mammalian transcription unit composed of a promoter flanked 3′ by a polylinker, an intron, and a transcriptional termination signal which is linked to a pVAX1 backbone. To improve expression, the Woodchuck Hepatitis Virus Posttranscriptional Regulatory Element (WPRE) was inserted at position 7 to 595 bp of pIDV-I thereby generating pIDV-II.

The pIDV-II vector was are used to generate DNA vector expressing antigens from the Crimean-Congo Hemorrhagic Fever virus (CCHF). Exemplary genes encoding CCHF antigens are provided in SEQ ID NOs:3-6 and SEQ ID NO:14 and are individually cloned into the vectors. The CCHF virus glycoproteins of SEQ ID NO:7-8 are derived from the CCHFV strain “Turkey”. The CCHF virus glycoprotein of SEQ ID NO:15 is from CCHF Turkey isolate 812955.

Experiments are performed to evaluate the cellular and humoral immune responses to the CCHF virus antigens in animals vaccinated with the DNA vectors.

The safety of the vaccine is determined by monitoring the systemic and local reaction to vaccination including site reactions and their resolution and clinical observation of the animals. Gross pathology will be performed at the end of the study.

The humoral response is determined using ELISA assay and the cellular response is determined by ELISPOT.

### Sample Size

For pre-clinical studies 8 groups of 10 female BALB/c mice aged between 6 to 8 weeks are used. Four (4) mice are tested for T-cell response and 6 for humoral immune response.

### Vaccination Dose and Prime Boost Schedule

In order to induce cellular and humoral immune response in mice, the DNA vaccines (pIDV-CCHF-GP-Tkk06-1, pIDV-CCHF-GP-Tkk06-2 (cocktail of pIDV-CCHF-Gn, pIDV-CCHF-Gc and pIDV—CCHF-NP); and empty backbone pIDV-Control) are administered by intramuscular injection.

Using this approach, the DNA vaccines are delivered to muscles by primary vaccination series followed by optional booster vaccination, i.e., entire dose of 200 μg is injected by two consecutive administrations into the exterior side of the mouse hind limbs. The volume and concentration of each injection is determined at 1 μg/μl or 100 μg/100 μl. The vaccine is administrated with 1 ml insulin syringes under isoflurane anesthesia, thus minimizing the puncture injury.

A baseline blood sample is collected from each mouse on Day −7 (in relation to the first dose of vaccine). Mice are vaccinated on Days 0 and 28 (see schedule of events table). For testing the humoral immune response, mice are bled on Days 7, 14, 21, 27, 35, 49. Samples for humoral and cellular analysis are also obtained on Days 38 and 56 when mice are sacrificed. One seronegative animal serves as a control in each group in which the empty DNA vector is administrated without prime boosting.

TABLE 1
Schedule of Events

| | Day −7 | Day 0 | Day 7 | Day 14 | Day 21 | Day 27 | Day 28 | Day 35 | Day 38 | Day 49 | Day 56 |
|---|---|---|---|---|---|---|---|---|---|---|---|
| Vaccination | | X | | | | | X | | | | |
| Bleed | X | | X | X | X | X | | X | | X | |
| Sacrifice | | | | | | | | | X* | | X^{#} |

*Four mice from each group are sacrificed for cellular immune response analysis
^{#}All remaining mice are sacrificed for humoral immune response analysis at the end of the study

Four out of 10 mice are anesthetized and then euthanized 10 days after boost vaccination by cardiac puncture, and their spleen is removed to compare the T cell response against the CCHF antigens in the different groups.

The 6 remaining mice are euthanized by cardiac puncture followed by cervical dislocation 28 days after the boost vaccination (i.e., 56 days after first vaccination).

The serum samples obtained at the different intervals (−7, 7, 14, 21 & 27) are used to evaluate the production of antibodies against the CCHF GP and NP in the different groups.

The DNA vaccines are tested in farming animals according to a similar protocol.

### Example 2

The pIDV-II plasmid (SEQ ID NO:12) was used to generate vaccines expressing CCHF antigens.

More particularly the codon optimized sequence set forth in SEQ ID NO:14 which expresses SEQ ID NO:15 was cloned into pIDV-II. This codon optimized sequence is 74% identical to the wild type Turkey isolate 812955 nucleic acid sequence (Accession number KY362519.1). The resulting DNA vaccine is referred to as pIDV—II-CCHF-GP (SEQ ID NO:13).

The pIDV—II-CCHF-GP (SEQ ID NO:13) expresses the full length of whole CCHFV M segment ORF obtained from NCBI GenBank (Turkey isolate 812955; segment M, complete sequence Accession number KY362519.1). Prior to cloning into the pIDV-II vector the glycoprotein was human codon-optimized and fused to the signal sequence of Kozak followed by the first methionine of antigen at the 3′ amino-terminus situated after the plasmid promoter. To this end, the CCHF-GP from pUC57 vector (GeneScript) was amplified using a primer pair with at least of 19 bp homology to the pIDV-II plasmid. The insert was gel-eluted and further inserted into pIDV-II backbone cut by Kpn-BglII at position 4613-9688 by Gibson Assembly protocol (New England Biolabs NEB).

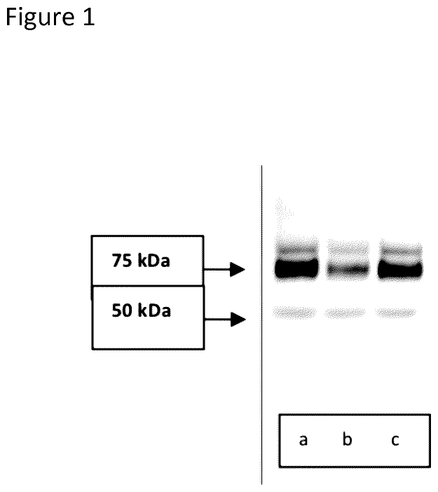

In order to compare the level of expression, the antigens were cloned in a similar fashion in two other plasmids: pVAX1 (SEQ ID NO:2) and pCAGGS as control groups. Antigen expression from the pVAX1 and pCAGGS vectors was compared by Western Blot (FIG. 1). The pIDV-II and pVAX1 vectors containing antigens were used in in vivo experiment.

An optimized DNA sequence encoding the full length of entire M segment of CCHF (CCHFV-GP-Turkey protein sequence set forth in SEQ ID NO:15) was cloned into the pIDV-II vector and was used for vaccination of sheep. This sequence is 86% identical to SEQ ID NO:14 (over the entire length of SEQ ID NO:14) and 74% identical to the wild type Turkey isolate 812955 nucleic acid sequence (Accession number KY362519.1). The resulting DNA vaccine is referred to as CCHFV-M DNA vaccine.

### Chemically Competent Cells Transformation

A 30 μl of chemically competent cells (Clontech Laboratories, Inc.) were thawed on ice for about 5 minutes and 3 μl of diluted assembled product was added to competent cells, gently mixed and incubated on ice for 30 minutes. Heat shock was performed at 42° C. for 45 seconds followed incubation on ice for 2 minutes. A 850 μl of SOC media at room temperature was added and the tube was placed at 37° C. for 60 minutes of incubation at 250 rpm. Selection plate was warmed in advance to 37° C. After an incubation 100 μl of the cells were spread by sterile loop onto the into the LB bacterial agar plate containing 50 mg/ml Neo/Kanamicine selective marker. Plates were incubated for overnight at 37° C.

### Screening of Single Clones for Absence of Mutations

Ten single clones from transformed bacterial colonies were chosen and grown in shakers for 14-16 hours at +37° C., 250 rpm into 5 ml of LB medium supplemented with 50 mg/ml Neo/Kan antibiotics. After incubation, transformants were harvested by centrifugation at 6000 g for 10 minutes. Plasmid DNA Mini prep purification was performed by QIAGEN Plasmid Mini Prep kit. Nucleic acids were quantified by NanoDrop 2000 (Thermo Scientific) prior to sequencing. Enzymatic digestion with restriction enzymes and gel electrophoresis (1% by AGE) were used to confirm the identity of the vectors.

To exclude that no spontaneous mutations in the transgene has been introduced, selected clones were submitted for nucleotide sequencing.

Sequencing primers for all experiment were designed using a 19-25 nt overlap with a Tm equal to or greater than 56° C. (assuming A-T pair=2° C. and G-C pair=4° C.) and have a GC content of about 50%.

The concentration of oligonucleotides was adjusted at 1.6 μM and the concentration of plasmid a ≈50 ng/μ; and submitted for Sanger sequencing. The plasmids having the best results of sequencing, especially for the absence of mutation, were selected for further evaluation of eGFP and for Western Blot respectively.

### Western Blot

At 24 h post-transfection, cell extracts were prepared in 50 mM Tris/HCl (pH 7.4), 5 mM EDTA, 1% Triton X-100 and Complete Protease Inhibitor cocktail. Cell lysates were centrifuged at 10 000 g for 10 min. The supernatant was quantified and 15 ug of each sample was mixed with sample buffer (10 M Tris/HCl (pH 6.8), 2% SDS, 10% glycerol, 5% β-mercaptoethanol, 0.005% bromophenol blue) and incubated at 56° C. for 10 min before electrophoresis in a Criterion Gel.

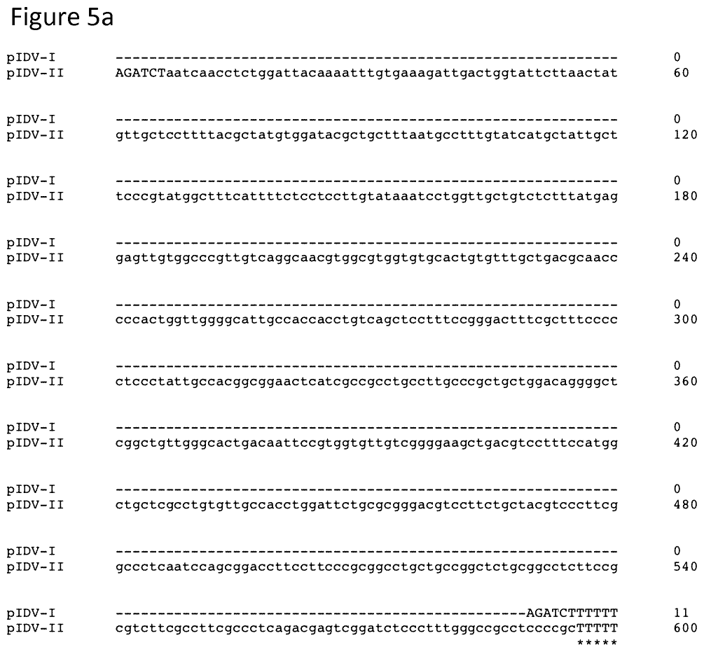

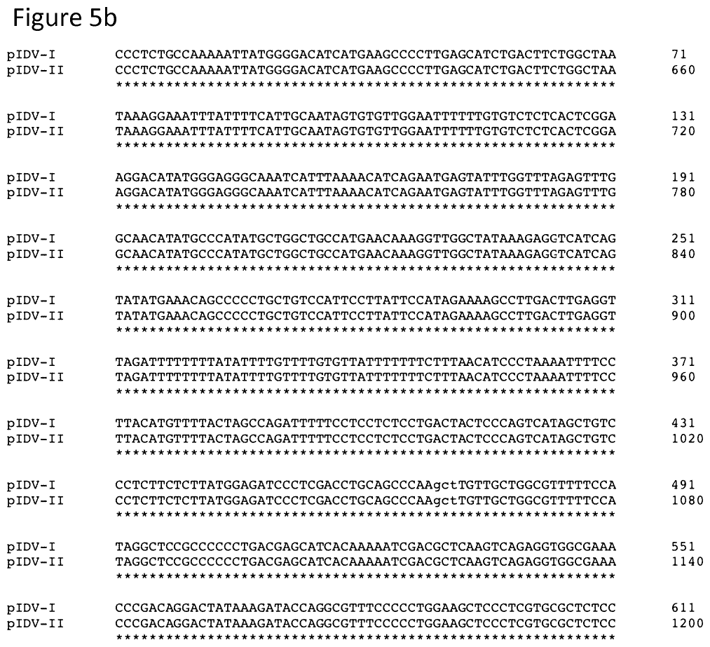

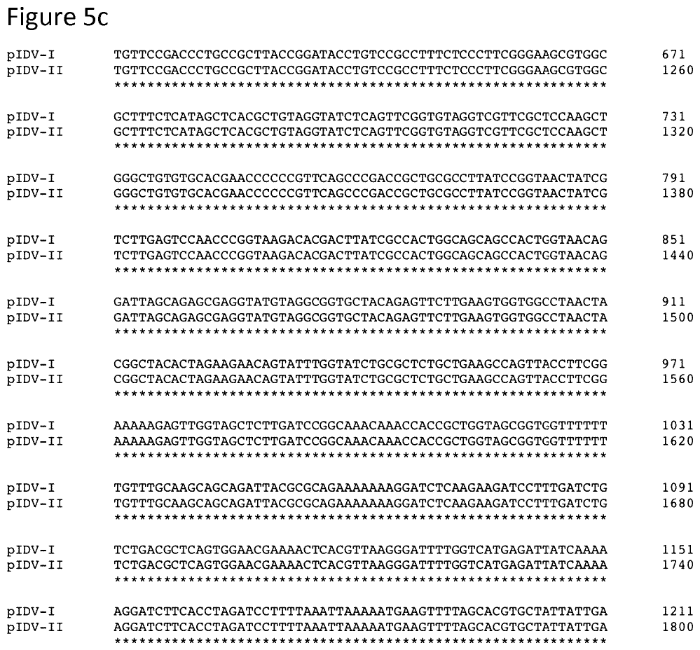

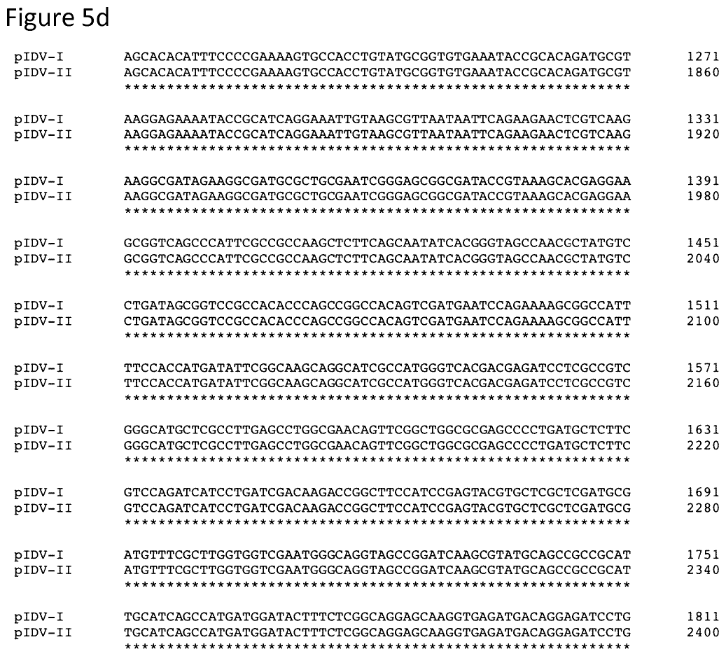

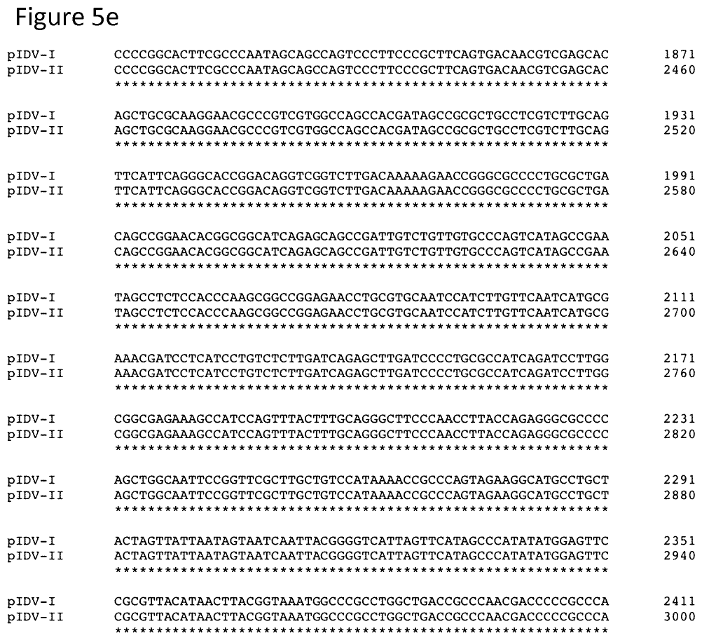

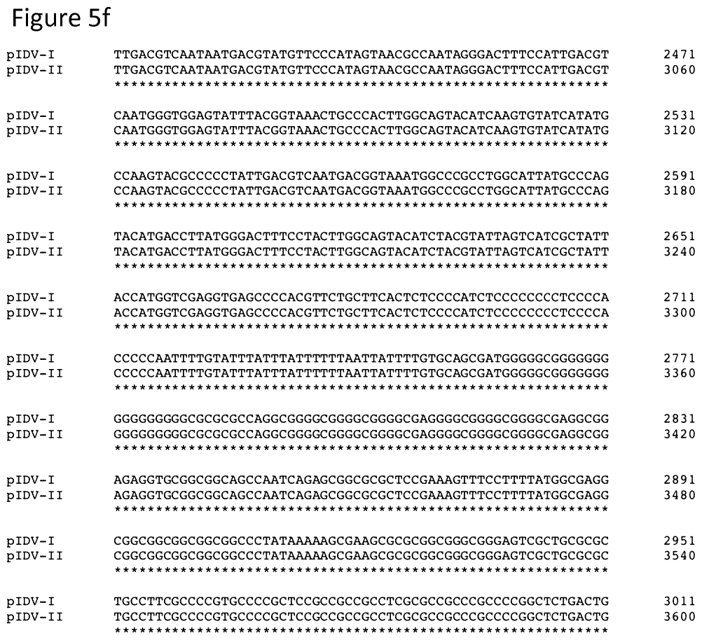

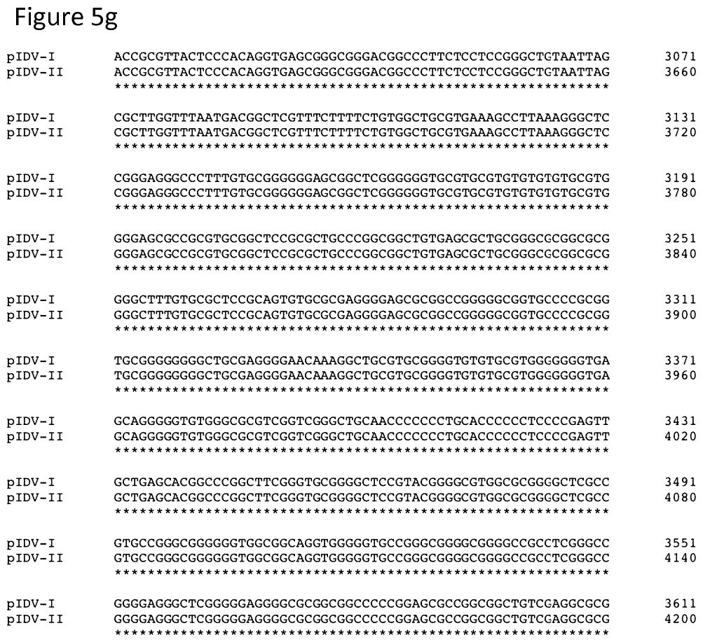

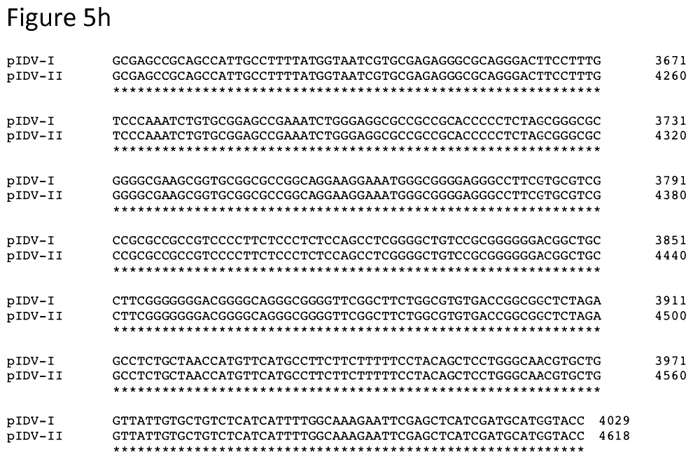

Western blot analysis was performed by using anti-CCHF mAb 11E7 (as primary antibodies for pre-GC-GCCCHF and incubated overnight at 4° C. with gentle agitation. As the loading control 1:20000 of secondary anti-a-Tubulin antibody (Sigma Aldrich) was used for each sample. Prior to adding the antibodies 3× washing steps were performed with 1×PBS-Tween 0.1% for 20, 5 and 5 minutes respectively. Goat anti-mouse human peroxidase-conjugated antibody was used followed by visualization with 4 ml total of substrate (Western blotting detection reagents Bio-Rad), while for HA86 containing backbone-Mouse IgG (H+L) Antibody, Human Serum Adsorbed and Peroxidase-Labeled antibody was used diluted at 1/20000. Results of protein expression are presented in FIGS. 5-8.

### Immunization of Mice

Groups of 7-10 mice aged 6-8 weeks (Charles River, Canada) were injected intramuscularly (IM) into the caudal thigh with 100 μg of pIDV-II and pVAX1 DNA vaccines containing the same antigen per animal diluted in Endotoxin-free TE buffer. Control animals received an equivalent volume of Endotoxin-free TE buffer. A total volume of 100 μl was introduced to each animal at two sites, each with 50 μl per limb. All mice were vaccinated with a single dose. Blood was obtained via subvein bleeds at day 0.14 and 21 until the euthanasia (day 28). Serum was separated and kept frozen until analyzed. Three mice from each group were euthanized at day 10 for analysis of T-cell response.

### Immunization of Sheep

Two groups of 2-3 months old 4 sheep were vaccinated by intramuscular injection in the semitendinosus muscle with either 1 mg of an optimized CCHFV-M DNA vaccine (encoding CCHFV-GP-Turkey protein sequence SEQ ID NO:15) (G1) or with Tris EDTA (TE) buffer (Control Group). No anaesthesia was required. Animals were vaccinated two times with prime boost at day 28. Blood was obtained every 7 days via jugular vein from day 28 to day 56. Serum was collected and kept frozen until analyzed. Each animal was health monitored and the data related to behavior and food intake was recorded daily. No health issues were observed throughout the entire experiment.

After prime boost CCHFV-specific antibodies were detected by ELISA against the CCHF VLPs as indicated herein.

### Mice Interferon-Gamma (IFN-γ) ELISpot Assay

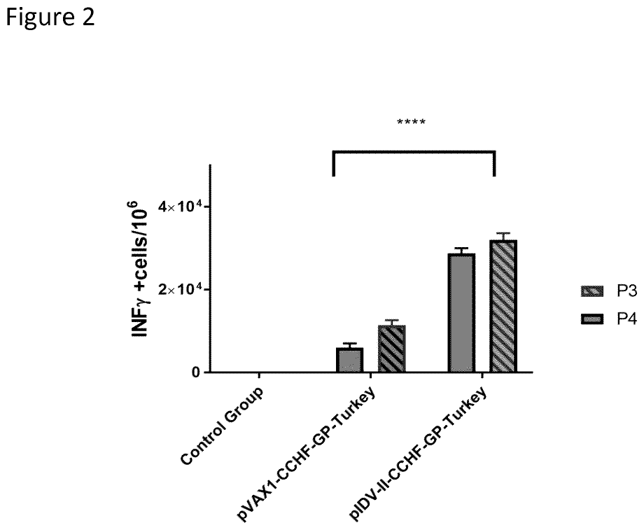

Splenocytes were assessed for CCHF antigen responses via IFN-γ enzyme-linked immunospot (ELISPOT) assay in accordance with manufacturer's instructions (BD Bioscience, San Jose, Calif.). Briefly, 96-well ELISPOT plates (Millipore, Billerica, Mass.) were coated overnight with anti-mouse interferon γ (IFN-γ) Ab, washed with phosphate-buffered saline, and blocked with 10% fetal bovine serum (FBS) in Roswell Park Memorial Institute medium (RPMI 1640). On day 10, splenocytes were harvested from 3 mice of each group of vaccinated mice to assess T-cell responses. A total of 5×10⁵ splenocytes in RPMI 10% FBS, 1% Pen/Strep and L-glutamine were plated per well and stimulated for 18-24 hours with 1 μg/mL of a peptide pools: for CCHF, partially overlapping peptide pools spanning the Gn and Gc of the CCHFV glycoprotein were applied in pools of 82 and 77 peptides designated as P3 and P4. 1% DMSO in RPMI and PMA 10 ng/ml/500 ng ionomicyn in RPMI was used as negative and positive controls respectively. Plates were placed for overnight incubation at 37° C. in a humidified incubator supplemented with 5% CO2. The following day, samples were extensively washed before incubation with biotinylated anti-mouse IFN-γ Ab. After incubation with streptavidin-horseradish peroxidase (HRP), IFN-γ-secreting cells were detected using AEC Chromogen (BD biosciences). Finally, spots were counted with an automated AID EliSpot Reader (FIG. 2).

### ELISA CCHF

CCHF Viral like Particles (CCHF VLPs) were made as a reagent for ELISA. To that effect, production of IbAr 10200 strain of CCHF VLPs was performed based on improved protocol previously reported by Garrison et al (PLoS Negl Trop Dis, 11(9): e0005908, 2017).

Briefly, HEK 293T cells were propagated to 70±80% confluency in 10 cm² round tissue culture plates and then transfected with 10 μg pC-M Opt (IbAr 10200), 4 μg pC-N, 2 μg L-Opt, 4 μg T7-Opt, and 1 μg Nano-luciferase encoding minigenome plasmid using the Promega FuGENE HD transfection reagent according to manufacturer's instructions (Thermo Fisher Scientific). Three days post-transfection, supernatants were harvested, cleared of debris, and VLPs were pelleted through a cushion of 20% sucrose in virus resuspension buffer (VRB; 130 mM NaCl, 20 mM HEPES, pH 7.4) by centrifugation for 2 h at 106,750×g in an SW32 rotor at 4° C. VLPs were resuspended overnight in 1/200 volume VRB at 4° C., and then frozen at −80° C. in single-use aliquots. Individual lots of CCHF-VLP were standardized.

Mice sera were collected 28 days post-vaccination. Flat bottom ELISA plates were coated overnight at 4° C. with approximately 1 ng N equivalent of CCHF-VLP diluted in 1×PBS per 96-well plate. The following day, plates were washed and then blocked with 3% PBS/BSA 2 h at 37° C. All washes were done with 1×PBS containing 0.1% Tween-20. Plates were washed again, prior to being loaded with two different dilutions of mice sera in duplicate (dilution range 1:200 and 1:800). Serum dilutions were carried out in blocking buffer. Plates were incubated at 37° C. for 80 minutes prior to being washed again, and then incubated with a 1:4000 dilution of horse radish peroxidase (HRP) conjugated rabbit anti-mouse (Mandel) in PBST for 80 minutes at 37° C. Plates were washed again and then developed with TMB substrate (Sera-Care Inc.). Absorbance at 450 nm wavelength was measured with a microplate reader. Individual naïve sheep sera for each group collected from the same day point was used as an internal control on each assay group. A plate cut-off value was determined based on the average absorbance of the naïve control starting dilution plus standard deviation. Only sample dilutions whose average was above this cut-off were registered as positive signal.

### Software

Statistical significance of total IgG/avidity ELISA data was determined using two-way (Sidak's post hoc correction) ANOVA test for CCHF. Significance levels were set at a P value less than 0.05. All analyses were performed using GraphPad Prism software (La Jolla, USA), version 7.04.

### Results

### T-Cell Response in Vaccinated Mice

IFN-γ ELISpot responses from Balb/c mice immunized with pIDV—II-CCHF-GP-Turkey are compared to that of pVAX1-CCHF-GP-Turkey. Splenocytes from vaccinated mice were activated with peptide pools derived from GP of IbAr 10200 strain of CCHF peptide pool 3 (detecting GN) and peptide pool 4 (detecting Gc). Patterned bars denote the number of spots against the peptide pool 3 while open bars show spot number against peptide pool 4 respectively. As can be seen from FIG. 2, animals vaccinated with pIDV—II-CCHF-GP-Turkey show higher T-cell response pattern compared to mice vaccinated with pVAX1 containing the same antigen. Results shown are the mean number of spot forming cells (SFC)±SD for 3 animals/group. Asterisks indicate statistically significant differences (****, p<0.005) (FIG. 2).

### Humoral Response at Day 0-28 Post Vaccination

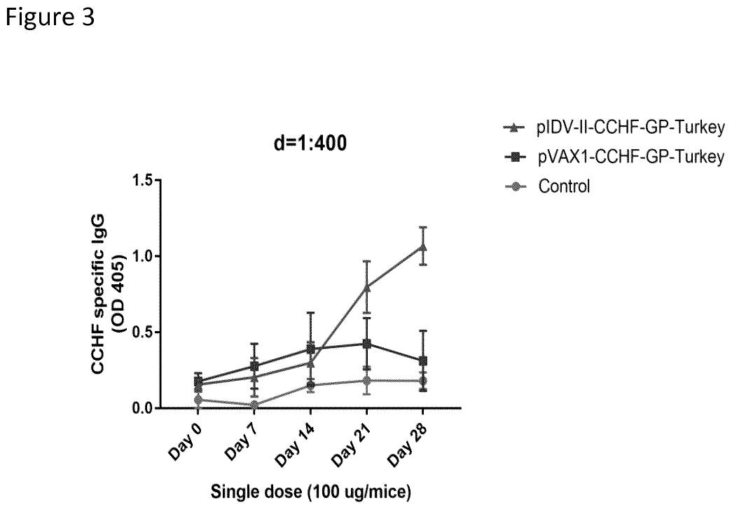

Results of FIG. 3 shows that only mice immunized with pIDV—II-CCHFV-GP developed IgG1 response with single dose. After single vaccination via IM route, CCHFV-specific antibodies were detected by ELISA against the CCHF-VLP only for mice vaccinated with pIDV—II-CCHF-GP-Turkey, while mice vaccinated with pVAX1-CCHF-GP-Turkey did not developed CCHF-specific antibodies. The CCHFV-specific IgG is shown in grouped mice following single vaccinations of 100 μg/mouse. Collected sera at 7 days intervals from Balb/c mice vaccinated with only Endofree TE buffer (Control group) were tested concurrently and had no detectable signal. For mice immunized with pIDV—II-CCHF-GP-Turkey the highest serum titer was observed at day 28 after immunization. *Two-way ANOVA, confidence intervals were set to 95%, P-value=<0.0001.

### Immune Response in Vaccinated Sheep

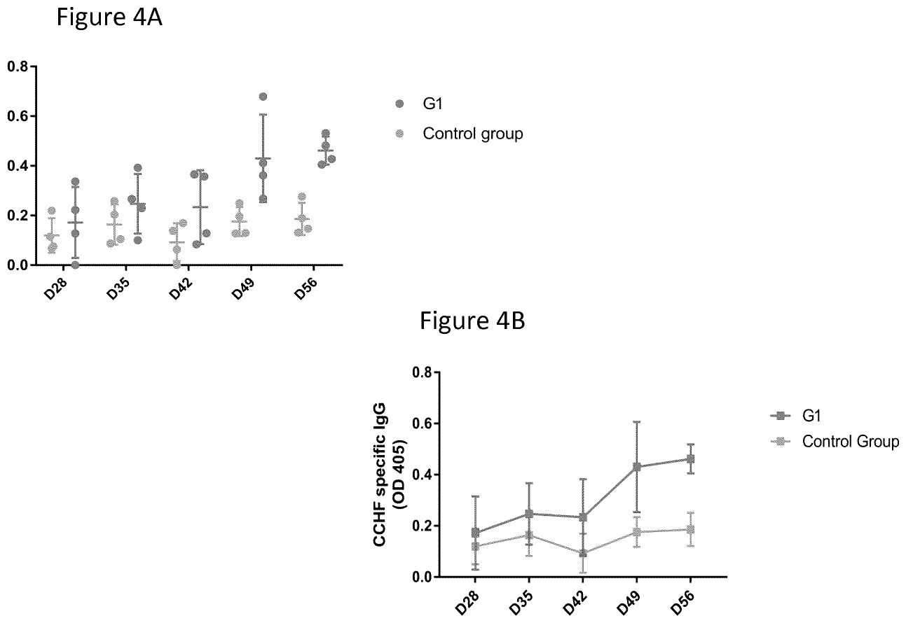

The level of CCHFV-specific IgGs in individual sheep (FIG. 4A) and grouped (FIG. 4B) was measured at day 28 and at 7 days intervals. Sheep vaccinated with buffer were tested concurrently and had no detectable signal. The CCHFV-specific antibody response was measured in vaccinated animals.

In sheep, the CCHFV-specific IgG ELISA titers of vaccinated animals significantly increased between the first and second vaccinations are particularly high at day 49. The results indicate that the vaccine generates a strong humoral response in sheep.

The results disclosed herein show that the DNA encoding CCHF GP triggers cell-mediated and humoral immune responses in mouse models with fully functional innate immunity. Advantageously only a single dose of vaccine was necessary, and the presence of helper vaccines was not required in these experiments.

Moreover, the results disclosed herein show that the CCHFV-GP DNA vaccine was highly immunogenic sheep.

Finally, an immune response was generated by intramuscular administration of naked DNA in both mice and sheep models.

SEQUENCE TABLE

| SEQ | | |
|---|---|---|
| ID | | |
| NO: | Description | Comment |
| 1 | pIDV plasmid nucleotide | BglII restriction site: |
| | sequence | nucleotides 1-6; |
| | | KpnI restriction site: |
| | | nucleotides 4094-4099 57% |
| | | GC |
| 2 | pVAX1 ™ plasmid sequence | |
| 3 | Crimean-Congo Hemorrhagic | |
| | Fever Virus glycoprotein | |
| | precursor (CCHF GP-Turkey- | |
| | kk06) | |
| 4 | Ubiquitin- CCHF Glycoprotein | Ubiquitin sequence corresponds |
| | GC | to nucleotide 1-228 |
| 5 | Ubiquitin- CCHF Glycoprotein | Ubiquitin sequence corresponds |
| | Gn | to nucleotide 1-228 |
| 6 | CCHF Nucleoprotein (NP) | |
| 7 | Crimean-Congo Hemorrhagic | Encoded by SEQ ID NO: 3 |
| | Fever Virus glycoprotein | |
| | precursor (CCHF GP-Turkey- | |
| | kk06) amino acid sequence | |
| 8 | Ubiquitin- CCHF Glycoprotein | Encoded by SEQ ID NO: 4 |
| | GC amino acid sequence | Ubiquitin sequence corresponds |
| | | to amino acid 1-76 |
| 9 | Ubiquitin- CCHF Glycoprotein | encoded by SEQ ID NO: 5 |
| | Gn amino acid sequence | Ubiquitin sequence corresponds |
| | | to amino acid 1-76 |
| 10 | CCHF Nucleoprotein (NP) - | Encoded by SEQ ID NO: 6 |
| | amino acid sequence | |
| 11 | pIDV-I plasmid nucleotide | |
| | sequence | |
| 12 | pIDV-II plasmid nucleotide | WPRE position 7-595 |
| | sequence | |
| 13 | pIDV-II-CCHF-GP-Turkey | CCFH Turkey antigen located at |
| | nucleotide sequence | position 4613-9688 |
| 14 | CCHF GP-Turkey nucleotide | |
| | sequence | |
| 15 | CCHF GP-Turkey amino acid | Encoded by SEQ ID NO: 13 and |
| | sequence | 14 |

A Sequence Listing in the form of a text file (entitled “16100_004_USPrv2_ST25_SequenceListing”, created on May 21, 2019 of 97 kilobytes) is incorporated herein by reference in its entirety.

SEQ ID NO.: 1: pIDV plasmid
AGATCTTTTTCCCTCTGCCAAAAATTATGGGGACATCATGAAGCCCCTTGAGCATCTGACTTCT
GGCTAATAAAGGAAATTTATTTTCATTGCAATAGTGTGTTGGAATTTTTTGTGTCTCTCACTCG
GAAGGACATATGGGAGGGCAAATCATTTAAAACATCAGAATGAGTATTTGGTTTAGAGTTTGGC
AACATATGCCCATATGCTGGCTGCCATGAACAAAGGTTGGCTATAAAGAGGTCATCAGTATATG
AAACAGCCCCCTGCTGTCCATTCCTTATTCCATAGAAAAGCCTTGACTTGAGGTTAGATTTTTT
TTATATTTTGTTTTGTGTTATTTTTTTCTTTAACATCCCTAAAATTTTCCTTACATGTTTTACT
AGCCAGATTTTTCCTCCTCTCCTGACTACTCCCAGTCATAGCTGTCCCTCTTCTCTTATGGAGA
TCCCTCGACCTGCAGCCCAAgctTGTTGCTGGCGTTTTTCCATAGGCTCCGCCCCCCTGACGAG
CATCACAAAAATCGACGCTCAAGTCAGAGGTGGCGAAACCCGACAGGACTATAAAGATACCAGG
CGTTTCCCCCTGGAAGCTCCCTCGTGCGCTCTCCTGTTCCGACCCTGCCGCTTACCGGATACCT
GTCCGCCTTTCTCCCTTCGGGAAGCGTGGCGCTTTCTCATAGCTCACGCTGTAGGTATCTCAGT
TCGGTGTAGGTCGTTCGCTCCAAGCTGGGCTGTGTGCACGAACCCCCCGTTCAGCCCGACCGCT
GCGCCTTATCCGGTAACTATCGTCTTGAGTCCAACCCGGTAAGACACGACTTATCGCCACTGGC
AGCAGCCACTGGTAACAGGATTAGCAGAGCGAGGTATGTAGGCGGTGCTACAGAGTTCTTGAAG
TGGTGGCCTAACTACGGCTACACTAGAAGAACAGTATTTGGTATCTGCGCTCTGCTGAAGCCAG
TTACCTTCGGAAAAAGAGTTGGTAGCTCTTGATCCGGCAAACAAACCACCGCTGGTAGCGGTGG
TTTTTTTGTTTGCAAGCAGCAGATTACGCGCAGAAAAAAAGGATCTCAAGAAGATCCTTTGATC
TGTCTGACGCTCAGTGGAACGAAAACTCACGTTAAGGGATTTTGGTCATGAGATTATCAAAAAG
GATCTTCACCTAGATCCTTTTAAATTAAAAATGAAGTTTTAGCACGTGCTATTATTGAAGCATT
TATCAGGGTTATTGTCTCATGAGCGGATACATATTTGAATGTATTTAGAAAAATAAACAAATAG
GGGTTCCGCGCACATTTCCCCGAAAAGTGCCACCTGTATGCGGTGTGAAATACCGCACAGATGC
GTAAGGAGAAAATACCGCATCAGGAAATTGTAAGCGTTAATAATTCAGAAGAACTCGTCAAGAA
GGCGATAGAAGGCGATGCGCTGCGAATCGGGAGCGGCGATACCGTAAAGCACGAGGAAGCGGTC
AGCCCATTCGCCGCCAAGCTCTTCAGCAATATCACGGGTAGCCAACGCTATGTCCTGATAGCGG
TCCGCCACACCCAGCCGGCCACAGTCGATGAATCCAGAAAAGCGGCCATTTTCCACCATGATAT
TCGGCAAGCAGGCATCGCCATGGGTCACGACGAGATCCTCGCCGTCGGGCATGCTCGCCTTGAG
CCTGGCGAACAGTTCGGCTGGCGCGAGCCCCTGATGCTCTTCGTCCAGATCATCCTGATCGACA
AGACCGGCTTCCATCCGAGTACGTGCTCGCTCGATGCGATGTTTCGCTTGGTGGTCGAATGGGC
AGGTAGCCGGATCAAGCGTATGCAGCCGCCGCATTGCATCAGCCATGATGGATACTTTCTCGGC
AGGAGCAAGGTGAGATGACAGGAGATCCTGCCCCGGCACTTCGCCCAATAGCAGCCAGTCCCTT
CCCGCTTCAGTGACAACGTCGAGCACAGCTGCGCAAGGAACGCCCGTCGTGGCCAGCCACGATA
GCCGCGCTGCCTCGTCTTGCAGTTCATTCAGGGCACCGGACAGGTCGGTCTTGACAAAAAGAAC
CGGGCGCCCCTGCGCTGACAGCCGGAACACGGCGGCATCAGAGCAGCCGATTGTCTGTTGTGCC
CAGTCATAGCCGAATAGCCTCTCCACCCAAGCGGCCGGAGAACCTGCGTGCAATCCATCTTGTT
CAATCATGCGAAACGATCCTCATCCTGTCTCTTGATCAGAGCTTGATCCCCTGCGCCATCAGAT
CCTTGGCGGCGAGAAAGCCATCCAGTTTACTTTGCAGGGCTTCCCAACCTTACCAGAGGGCGCC
CCAGCTGGCAATTCCGGTTCGCTTGCTGTCCATAAAACCGCCCAGTAGAAGGCATGCCTGCTAC
TAGTTATTAATAGTAATCAATTACGGGGTCATTAGTTCATAGCCCATATATGGAGTTCCGCGTT
ACATAACTTACGGTAAATGGCCCGCCTGGCTGACCGCCCAACGACCCCCGCCCATTGACGTCAA
TAATGACGTATGTTCCCATAGTAACGCCAATAGGGACTTTCCATTGACGTCAATGGGTGGAGTA
TTTACGGTAAACTGCCCACTTGGCAGTACATCAAGTGTATCATATGCCAAGTACGCCCCCTATT
GACGTCAATGACGGTAAATGGCCCGCCTGGCATTATGCCCAGTACATGACCTTATGGGACTTTC
CTACTTGGCAGTACATCTACGTATTAGTCATCGCTATTACCATGGTCGAGGTGAGCCCCACGTT
CTGCTTCACTCTCCCCATCTCCCCCCCCTCCCCACCCCCAATTTTGTATTTATTTATTTTTTAA
TTATTTTGTGCAGCGATGGGGGCGGGGGGGGGGGGGCCGCGCGCCAGCCGGGGCGGGGCGGGGC
GAGGGGCGGGGCGGGGCGAGGCGGAGAGGTGCGGCGGCAGCCAATCAGAGCGGCGCGCTCCGAA
AGTTTCCTTTTATGGCGAGGCGGCGGCGGCGGCGGCCCTATAAAAAGCGAAGCGCGCGGCGGGC
GGGAGTCGCTGCGTTGCCTTCGCCCCGTGCCCCGCTCCGCGCCGCCTCGCGCCGCCCGCCCCGG
CTCTGACTGACCGCGTTACTCCCACAGGTGAGCGGGCGGGACGGCCCTTCTCCTCCGGGCTGTA
ATTAGCGCTTGGTTTAATGACGGCTCGTTTCTTTTCTGTGGCTGCGTGAAAGCCTTAAAGGGCT
CCGGGAGGGCCCTTTGTGCGGGGGGAGCGGCTCGGGGGGTGCGTGCGTGTGTGTGTGCGTGGGG
AGCGCCGCGTGCGGCTCCGCGCTGCCCGGCGGCTGTGAGCGCTGCGGGCGCGGCGCGGGGCTTT
GTGCGCTCCGCAGTGTGCGCGAGGGGAGCGCGGCCGGGGGCGGTGCCCCGCGGTGCGGGGGGGG
CTGCGAGGGGAACAAAGGCTGCGTGCGGGGTGTGTGCGTGGGGGGGTGAGCAGGGGGTGTGGGC
GCGTCGGTCGGGCTGCAACCCCCCCTGCACCCCCCTCCCCGAGTTGCTGAGCACGGCCCGGCTT
CGGGTGCGGGGCTCCGTACGGGGCGTGGCGCGGGGCTCGCCGTGCCGGGCGGGGGGTGGCGGCA
GGTGGGGGTGCCGGGCGGGGCGGGGCCGCCTCGGGCCGGGGAGGGCTCGGGGGAGGGGCGCGGC
GGCCCCCGGAGCGCCGGCGGCTGTCGAGGCGCGGCGAGCCGCAGCCATTGCCTTTTATGGTAAT
CGTGCGAGAGGGCGCAGGGACTTCCTTTGTCCCAAATCTGTGCGGAGCCGAAATCTGGGAGGCG
CCGCCGCACCCCCTCTAGCGGGCGCGGGGCGAAGCGGTGCGGCGCCGGCAGGAAGGAAATGGGC
GGGGAGGGCCTTCGTGCGTCGCCGCGCCGCCGTCCCCTTCTCCCTCTCCAGCCTCGGGGCTGTC
CGCGGGGGGACGGCTGCCTTCGGGGGGGACGGGGCAGGGCGGGGTTCGGCTTCTGGCGTGTGAC
CGGCGGCTCTAGAGCCTCTGCTAACCATGTTCATGCCTTCTTCTTTTTCCTACAGCTCCTGGGC
AACGTGCTGGTTATTGTGCTGTCTCATCATTTTGGCAAAGAATTCGAGCTCATCGATGCATGGT
ACC
SEQ ID NO.: 2: pVAX1 ™ plasmid sequence
GACTCTTCGCGATGTACGGGCCAGATATACGCGTTGACATTGATTATTGACTAGTTATTAATAG
TAATCAATTACGGGGTCATTAGTTCATAGCCCATATATGGAGTTCCGCGTTACATAACTTACGG
TAAATGGCCCGCCTGGCTGACCGCCCAACGACCCCCGCCCATTGACGTCAATAATGACGTATGT
TCCCATAGTAACGCCAATAGGGACTTTCCATTGACGTCAATGGGTGGACTATTTACGGTAAACT
GCCCACTTGGCAGTACATCAAGTGTATCATATGCCAAGTACGCCCCCTATTGACGTCAATGACG
GTAAATGGCCCGCCTGGCATTATGCCCAGTACATGACCTTATGGGACTTTCCTACTTGGCAGTA
CATCTACGTATTAGTCATCGCTATTACCATGGTGATGCGGTTTTGGCAGTACATCAATGGGCGT
GGATAGCGGTTTGACTCACGGGGATTTCCAAGTCTCCACCCCATTGACGTCAATGGGAGTTTGT
TTTGGCACCAAAATCAACGGGACTTTCCAAAATGTCGTAACAACTCCGCCCCATTGACGCAAAT
GGGCGGTAGGCGTGTACGGTGGGAGGTCTATATAAGCAGAGCTCTCTGGCTAACTAGAGAACCC
ACTGCTTACTGGCTTATCGAAATTAATACGACTCACTATAGGGAGACCCAAGCTGGCTAGCGTT
TAAACTTAAGCTTGGTACCGAGCTCGGATCCACTAGTCCAGTGTGGTGGAATTCTGCAGATATC
CAGCACAGTGGCGGCCGCTCGAGTCTAGAGGGCCCGTTTAAACCCGCTGATCAGCCTCGACTGT
GCCTTCTAGTTGCCAGCCATCTGTTGTTTGCCCCTCCCCCGTGCCTTCCTTGACCCTGGAAGGT
GCCACTCCCACTGTCCTTTCCTAATAAAATGAGGAAATTGCATCGCATTGTCTGAGTAGGTGTC
ATTCTATTCTGGGGGGTGGGGTGGGGCAGGACAGCAAGGGGGAGGATTGGGAAGACAATAGCAG
GCATGCTGGGGATGCGGTGGGCTCTATGGCTTCTACTGGGCGGTTTTATGGACAGCAAGCGAAC
CGGAATTGCCAGCTGGGGCGCCCTCTGGTAAGGTTGGGAAGCCCTGCAAAGTAAACTGGATGGC
TTTCTCGCCGCCAAGGATCTGATGGCGCAGGGGATCAAGCTCTGATCAAGAGACAGGATGAGGA
TCGTTTCGCATGATTGAACAAGATGGATTGCACGCAGGTTCTCCGGCCGCTTGGGTGGAGAGGC
TATTCGGCTATGACTGGGCACAACAGACAATCGGCTGCTCTGATGCCGCCGTGTTCCGGCTGTC
AGCGCAGGGGCGCCCGGTTCTTTTTGTCAAGACCGACCTGTCCGGTGCCCTGAATGAACTGCAA
GACGAGGCAGCGCGGCTATCGTGGCTGGCCACGACGGGCGTTCCTTGCGCAGCTGTGCTCGACG
TTGTCACTGAAGCGGGAAGGGACTGGCTGCTATTGGGCGAAGTGCCGGGGCAGGATCTCCTGTC
ATCTCACCTTGCTCCTGCCGAGAAAGTATCCATCATGGCTGATGCAATGCGGCGGCTGCATACG
CTTGATCCGGCTACCTGCCCATTCGACCACCAAGCGAAACATCGCATCGAGCGAGCACGTACTC
GGATGGAAGCCGGTCTTGTCGATCAGGATGATCTGGACGAAGAGCATCAGGGGCTCGCGCCAGC
CGAACTGTTCGCCAGGCTCAAGGCGAGCATGCCCGACGGCGAGGATCTCGTCGTGACCCATGGC
GATGCCTGCTTGCCGAATATCATGGTGGAAAATGGCCGCTTTTCTGGATTCATCGACTGTGGCC
GGCTGGGTGTGGCGGACCGCTATCAGGACATAGCGTTGGCTACCCGTGATATTGCTGAAGAGCT
TGGCGGCGAATGGGCTGACCGCTTCCTCGTGCTTTACGGTATCGCCGCTCCCGATTCGCAGCGC
ATCGCCTTCTATCGCCTTCTTGACGAGTTCTTCTGAATTATTAACGCTTACAATTTCCTGATGC
GGTATTTTCTCCTTACGCATCTGTGCGGTATTTCACACCGCATACAGGTGGCACTTTTCGGGGA
AATGTGCGCGGAACCCCTATTTGTTTATTTTTCTAAATACATTCAAATATGTATCCGCTCATGA
GACAATAACCCTGATAAATGCTTCAATAATAGCACGTGCTAAAACTTCATTTTTAATTTAAAAG
GATCTAGGTGAAGATCCTTTTTGATAATCTCATGACCAAAATCCCTTAACGTGAGTTTTCGTTC
CACTGAGCGTCAGACCCCGTAGAAAAGATCAAAGGATCTTCTTGAGATCCTTTTTTTCTGCGCG
TAATCTGCTGCTTGCAAACAAAAAAACCACCGCTACCAGCGGTGGTTTGTTTGCCGGATCAAGA
GCTACCAACTCTTTTTCCGAAGGTAACTGGCTTCAGCAGAGCGCAGATACCAAATACTGTCCTT
CTAGTGTAGCCGTAGTTAGGCCACCACTTCAAGAACTCTGTAGCACCGCCTACATACCTCGCTC
TGCTAATCCTGTTACCAGTGGCTGCTGCCAGTGGCGATAAGTCGTGTCTTACCGGGTTGGACTC
AAGACGATAGTTACCGGATAAGGCGCAGCGGTCGGGCTGAACGGGGGGTTCGTGCACACAGCCC
AGCTTGGAGCGAACGACCTACACCGAACTGAGATACCTACAGCGTGAGCTATGAGAAAGCGCCA
CGCTTCCCGAAGGGAGAAAGGCGGACAGGTATCCGGTAAGCGGCAGGGTCGGAACAGGAGAGCG
CACGAGGGAGCTTCCAGGGGGAAACGCCTGGTATCTTTATAGTCCTGTCGGGTTTCGCCACCTC
TGACTTGAGCGTCGATTTTTGTGATGCTCGTCAGGGGGGCGGAGCCTATGGAAAAACGCCAGCA
ACGCGGCCTTTTTACGGTTCCTGGGCTTTTGCTGGCCTTTTGCTCACATGTTCTT
SEQ ID NO: 3: Crimean Congo Hemorrhagic Fever Virus glycoprotein
precursor (CCHF GP-Turkey-kk06)
ATGCCTACCAATATCATGCATACACCCCTCGTGTGCTTTATACTTTACCTCCAATTGTTGTGCT
TGGGTGGGGCCCACGGACAATTGAACGCCACCGAACACAATGGGACGAACAATACCACTGCTCC
CGGCGCTAGTCAATCTCCTAAACCTCCCATGAGCACCACGCCTCCACATGCGCCAGAATCATCA
ACAATCAAGCCCACGACACCTATCTCCGAGGCGGAGGGGTCAGGAGAGACTACGTCACCCCCGA
ATACCACGCAGGGCCTGTCTTCTCCGGAAACCACTTCCGAAAGGCCAGCAACTACGAGCATTAG
TACTAGCAGTACCGATTCCACGAACCCAACGACACAAATGACGGACAATACTCCTACGCCAACA
GTTAGTACATCCCCCAGCTCCAGTCCTTCAACCCCTAGTACTCCGCAGGGCATCCACCATCCAG
CGAGATCCCTCCTGTCTGTCAGTAGCCCAAAGACTGTCACGACACCAACGCCGACCTCTCCCGG
AGAGATGTCTTCTGAGACTTCTTCACAGCATAGCGCGATGTCAAGAATCCCGACTCCCCACACA
GCGACGCGCGTTTCAACAGAAATCACAAACCACCGGACTCCGCGACAATCTGAGTCATCTGCTC
AACAAACTACTCCTTCTCCTATGACGTCTCCTGCCCAGTCCATTTTGCTTATGAGCGCAGCTCC
AACTGCGGTCCAGGACATCCACCCTTCCCCTACTAATAGGTCCAAGCGAAACCTTGAGACAGAA
ATTATTCTCACACTCTCCCAGGGGTTGAAAAAATATTACGGCAAAATCCTGAAACTGTTGCATC
TGACCCTCGAGCAAGACACTGAAGGCCTCCTCGAGTGGTGTAAGGGTAATTTGGGCAGCAATTG
TGATGATGATTTCTTCCAAAAAAGGATCGAGGAATTCTTTATGACCGGCGAGTGCTATTTTAAT
GAGGTCCTTCAGTTCAAGACACTGAGTACCCTCTCACCTACCGAACCTTCACACGCCAGGCTTC
CGACAGCCGAACCTTTTAAAAGTTACTTCGCAAAGGGCTTTTTGAGTATCGACTCCGGATACTT
CAGTGCTAAGTGCTACCCTCGGTCAAGCGCATCCGGGCTCCAGTTGATTAATGTTACACAGCAT
CCCGCACGGATCGCAGAGACACCAGGTCCGAAAACTACTTCCTTGAAGACAATCAACTGCATTA
ACCTGCGAGCTTCCGTCTTCAAAGAGCATAGAGAAGTCGAAATAAATGTACTTCTCCCCCAGAT
CGCAGTTAACTTGTCAAATTGTCACGTCGTAATCAACAGCCATGTTTGTGACTATTCCCTTGAT
ACCGATGGTCCCGTTCGACTCCCTAGGATATACCACGAAGGAACATTCATACCGGGCACTTATA
AGATCGTAATCGATAAGAAGAATAAACTTAATGACCGGTGTACGCTGGTCACCAATTGCGTGAT
CAAAGGTAGAGAGGTGAGAAAAGGACAAAGTGTGTTGCGACAATATAAGACGGAAATCAAAATT
GGCAAGGCTTCCACGGGGTTCAGAAAACTGCTCAGTGAGGAGCCGGGCGACGACTGCATCTCCA
GGACGCAACTTTTGAGAACAGAGACAGCCGAGATTCATGATGATAATTATGGAGGACCTGGAGA
CAAAATAACGATATGTAACGGCTCCACTATAGTCGACCAAAGATTGGGAAGCGAGCTTGGTTGC
TACACGATTAACAGAGTGAAGTCTTTTAAACTTTGTAAGAACAGTGCCACGGGGAAGACATGTG
AAGTTGACTCCACCCCAGTGAAATGCCGACAAGGATTTTGTCTGAAGATTACACAAGAAGGTCG
AGGCCACGTCAAACTTTCAAGGGGAAGTGAAGTCGTTTTGGACGCTTGCGATTCATCCTGCGAA
GTAATGATTCCAAAAGGTACTGGAGACATTTTGGTGGATTGTTCAGGCGGGCAACAACACTTTC
TGAAAGACAACCTGATAGACCTCGGATGCCCTCATATCCCCCTGCTTGGTAGGATGGCAATCTA
TATCTGTCGGATGTCAAATCATCCTCGAACTACGATGGCATTCCTCTTTTGGTTCTCTTTTGGT
TACGTCATTACATGCATTTTCTGTAAGGCACTCTTTTATTCACTCATTATCATCGGGACACTTG
GAAAAAAAATCAAACAATACCGGGAACTTAAGCCCCAAACCTGCACCATTTGCGAAACTGCGCC
AGTAAACGCAATCGATGCCGAAATGCATGATCTCAATTGTTCATACAACATATGTCCCTACTGT
GCGAGCAGGCTTACTAGTGATGGTTTGGCACGACACGTAACTCAGTGTCCTAAGAGAAAAGAAA
AAGTCGAAGAAACAGAATTGTACCTCAACCTCGAACGAATACCTTGGATCGTAAGGAAGCTCCT
GCAGGTCTCAGAGAGTACCGGGGTCGCTCTTAAAAGAAGCTCATGGCTGATCGTACTTTTGGTC
CTCCTCACTGTATCACTGTCCCCTGTCCAAAGCGCCCCAGTCGGACATGGCAAAACTATTGAGA
TATATCAAACGCGAGAAGGGTTTGCATCTATTTGTCTTTTTATGCTCGGCAGCATCCTCTTCAT
AGTGTCTTGCTTGGTCAAGGGACTGGTAGACAGCGTGTCTGAATCTTTCTTTCCTGGGCTCTCA
GTCTGTAAGACTTGTTCCATTGGCAGTGTCAACGGTTTCGAGATTGAATCCCACAAGTGTTATT
GCAGTTTGTTTTGCTGCCCATACTGCCGCCATTGTAGCGCGGATCGAGAAATTCATCAACTGCA
CCTTAGCATTTGCAAAAAGAGGAAAACGGGCTCAAACGTAATGCTCGCGGTCTGCAAACGAATG
TGTTTCAGAGCGACAATTGAGGCAAGTCGGCGAGCACTGCTGATTAGGAGTATTATTAATACTA
CGTTTGTGATCTGTATCCTCACGCTGACTATATGCGTTGTCAGTACATCCGCCGTCGAGATGGA
AAATCTTCCTGCAGGGACGTGGGAGCGAGAGGAAGATCTTACTAATTTTTGCCATCAAGAATGT
CAAGTGACAGAAACTGAATGCTTGTGCCCCTATGAAGCCCTTGTGTTGAGGAAGCCACTCTTTC
TTGATTCTATAGTTAAGGGAATGAAAAATTTGCTCAACAGTACGAGTCTGGAAACCTCTTTGTC
AATCGAGGCCCCATGGGGTGCCATCAACGTGCAGAGTACGTTTAAGCCAACCGTTTCAACCGCT
AACATCGCTCTTAGCTGGTCAAGCGTCGTTCATAGAGGAAACAAGATACTTGTCACTGGACGGA
GTGAAAGCATCATGAAGCTTGAGGAAAGGACTGGGGTGAGTTGGGATCTTGGGGTCGAGGACGC
ATCTGAAAGTAAACTGTTGACGGTCTCTATCATGGATCTCTCACAGATGTACAGCCCGGTTTTT
GAGTACCTCAGCGGGGATCGACAAGTTGAGGAGTGGCCCAAGGCTACCTGCACCGGAGATTGCC
CGGAGCGATGCGGGTGCACTAGCTCAACATGTCTCCATAAGGAGTGGTCCCATAGCCGAAATTG
GAGGTGTAACCCCACTTGGTGCTGGGGTGTCGGCACAGGATGTACTTGTTGCGGAGTCGATGTA
AAAGACCTGTTCACGGACCATATGTTTGTCAAATGGAAGGTGGAGTACATTAAAACTGAAGCCA
TTGTGTGCGTTGCGCTTACGTCTCAAGAACGGCAATGTTCACTGATCGAAGCAGGGACTCGGTT
CAACTTGGGGCCTGTCACAATAACTTTGTCAGAGCCGAGAAACATACAACAGAAGCTTCCACCC
GAAATCATAACCTTGCATCCGAAAATAGAGGAAGGGTTCTTCGATCTTATGCACGTCCAAAAAG
TGCTTAGTGCCTCTACGGTCTGCAAACTCCAAAGCTGCACTCACGGTATCCCGGGGGATCTGCA
AGTTTACCACATAGGCAACCTTTTGAAAGGAGATAGGGTCAACGGCCACCTGATACATAAGATT
GAAAGCCATTTCAACACAAGTTGGATGAGTTGGGATGGCTGCGATTTGGATTATTACTGCAATA
TGGGGGACTGGCCCAGCTGTACATATACTGGAGTTACACAGCATAATCATGCGGCATTTGTTAA
CCTCCTTAACATCGAGACAGACTACACAAAGACCTTTCACTTCCACTCCAAGAGAGTGACTGCG
CACGGAGACACGCCCCAACTTGACCTTAAAGCTAGACCGACCTACGGAGCGGGCGGAATCACAG
TCCTGGTTGAAGTAGCTGATATGGAATTGCATACGAAAAAGGTTGAGATTAGTGGGCTCAAGTT
CGCGTCACTCGCTTGCACGGGTTGCTATGCGTGTTCCAGCGGCATTAGCTGCAAGGTCAGAATC
CATGTTGACGAGCCGGATGAGTTGACAGTACATGTCAAATCCAGTGACCCAGACGTTGTCGCTG
CAAGTACATCCCTGATGGCGAGGAAGCTGGAATTCGGCACGGACTCCACGTTCAAAGCGTTTTC
AGCGATGCCGAAGACGTCTTTGTGTTTCTATATTGTAGAGCGAGAATATTGTAAAAGCTGCAGT
GAAGATGACACTCAGAAATGCGTTGATACTCGCTTGGAACAGCCTCAGTCAATTCTCATAGAAC
ATAAGGGAACAATTATCGGAAAACAGAACGATACTTGTACCGCCAAAGCATCCTGTTGGCTGGA
AAGTGTGAAGTCTTTTTTCTATGGCCTGAAAAACATGCTTGGGAGTGTCTTCGGGAATTTGTTC
ATCGGCATACTGCTTTTCTTGGCCCCCTTTGTCCTCCTCGTCCTCTTCTTCATGTTCGGATGGA
AGATACTGTTTTGCTTCAAATGCTGCAGACGCACTAGGGGGCTGTTCAAGTATCGACATCTGAA
GGACGACGAAGAGACCGGGTACCGAAGGATTATCGAGAGACTCAATTCTAAAAAGGGCAAAAAT
CGGTTGCTTGACGGGGACCGCTTGGCAGACAGGAAGATCGCAGAGTTGTTCTCCACGAAAACAC
ATATCGGATAA
SEQ ID NO: 4: Ubiquitin-CCHF Glycoprotein GC in which the ubiquitin-
related sequence is underlined
ATGCAGATCTTCGTGAAAACCCTTACCGGCAAGACCATCACCCTTGAGGTGGAGCCCAGTGACA
CCATCGAAAATGTGAAGGCCAAGATCCAGGATAAGGAAGGCATTCCCCCCGACCAGCAGAGGCT
CATCTTTGCAGGCAAGCAGCTGGAAGATGGCCGTACTCTTTCTGACTACAACATCCAGAAGGAG
TCGACCCTGCACCTGGTCCTGCGTCTGAGAGGTGGTTTTCTTGATTCTATAGTTAAGGGAATGA
AAAATTTGCTCAACAGTACGAGTCTGGAAACCTCTTTGTCAATCGAGGCCCCATGGGGTGCCAT
CAACGTGCAGAGTACGTTTAAGCCAACCGTTTCAACCGCTAACATCGCTCTTAGCTGGTCAAGC
GTCGTTCATAGAGGAAACAAGATACTTGTCACTGGACGGAGTGAAAGCATCATGAAGCTTGAGG
AAAGGACTGGGGTGAGTTGGGATCTTGGGGTCGAGGACGCATCTGAAAGTAAACTGTTGACGGT
CTCTATCATGGATCTCTCACAGATGTACAGCCCGGTTTTTGAGTACCTCAGCGGGGATCGACAA
GTTGAGGAGTGGCCCAAGGCTACCTGCACCGGAGATTGCCCGGAGCGATGCGGGTGCACTAGCT
CAACATGTCTCCATAAGGAGTGGTCCCATAGCCGAAATTGGAGGTGTAACCCCACTTGGTGCTG
GGGTGTCGGCACAGGATGTACTTGTTGCGGAGTCGATGTAAAAGACCTGTTCACGGACCATATG
TTTGTCAAATGGAAGGTGGAGTACATTAAAACTGAAGCCATTGTGTGCGTTGCGCTTACGTCTC
AAGAACGGCAATGTTCACTGATCGAAGCAGGGACTCGGTTCAACTTGGGGCCTGTCACAATAAC
TTTGTCAGAGCCGAGAAACATACAACAGAAGCTTCCACCCGAAATCATAACCTTGCATCCGAAA
ATAGAGGAAGGGTTCTTCGATCTTATGCACGTCCAAAAAGTGCTTAGTGCCTCTACGGTCTGCA
AACTCCAAAGCTGCACTCACGGTATCCCGGGGGATCTGCAAGTTTACCACATAGGCAACCTTTT
GAAAGGAGATAGGGTCAACGGCCACCTGATACATAAGATTGAAAGCCATTTCAACACAAGTTGG
ATGAGTTGGGATGGCTGCGATTTGGATTATTACTGCAATATGGGGGACTGGCCCAGCTGTACAT
ATACTGGAGTTACACAGCATAATCATGCGGCATTTGTTAACCTCCTTAACATCGAGACAGACTA
CACAAAGACCTTTCACTTCCACTCCAAGAGAGTGACTGCGCACGGAGACACGCCCCAACTTGAC
CTTAAAGCTAGACCGACCTACGGAGCGGGCGGAATCACAGTCCTGGTTGAAGTAGCTGATATGG
AATTGCATACGAAAAAGGTTGAGATTAGTGGGCTCAAGTTCGCGTCACTCGCTTGCACGGGTTG
CTATGCGTGTTCCAGCGGCATTAGCTGCAAGGTCAGAATCCATGTTGACGAGCCGGATGAGTTG
ACAGTACATGTCAAATCCAGTGACCCAGACGTTGTCGCTGCAAGTACATCCCTGATGGCGAGGA
AGCTGGAATTCGGCACGGACTCCACGTTCAAAGCGTTTTCAGCGATGCCGAAGACGTCTTTGTG
TTTCTATATTGTAGAGCGAGAATATTGTAAAAGCTGCAGTGAAGATGACACTCAGAAATGCGTT
GATACTCGCTTGGAACAGCCTCAGTCAATTCTCATAGAACATAAGGGAACAATTATCGGAAAAC
AGAACGATACTTGTACCGCCAAAGCATCCTGTTGGCTGGAAAGTGTGAAGTCTTTTTTCTATGG
CCTGAAAAACATGCTTGGGAGTGTCTTCGGGAATTTGTTCATCGGCATACTGCTTTTCTTGGCC
CCCTTTGTCCTCCTCGTCCTCTTCTTCATGTTCGGATGGAAGATACTGTTTTGCTTCAAATGCT
GCAGACGCACTAGGGGGCTGTTCAAGTATCGACATCTGAAGGACGACGAAGAGACCGGGTACCG
AAGGATTATCGAGAGACTCAATTCTAAAAAGGGCAAAAATCGGTTGCTTGACGGGGACCGCTTG
GCAGACAGGAAGATCGCAGAGTTGTTCTCCACGAAAACACATATCGGATAA
SEQ ID NO: 5: Ubiquitin-CCHF Glycoprotein Gn in which the ubiquitin-
related sequence is underlined
ATGCAGATCTTCGTGAAAACCCTTACCGGCAAGACCATCACCCTTGAGGTGGAGCCCAGTGACA
CCATCGAAAATGTGAAGGCCAAGATCCAGGATAAGGAAGGCATTCCCCCCGACCAGCAGAGGCT
CATCTTTGCAGGCAAGCAGCTGGAAGATGGCCGTACTCTTTCTGACTACAACATCCAGAAGGAG
TCGACCCTGCACCTGGTCCTGCGTCTGAGAGGTGGTAGTGAGGAGCCGGGCGACGACTGCATCT
CCAGGACGCAACTTTTGAGAACAGAGACAGCCGAGATTCATGATGATAATTATGGAGGACCTGG
AGACAAAATAACGATATGTAACGGCTCCACTATAGTCGACCAAAGATTGGGAAGCGAGCTTGGT
TGCTACACGATTAACAGAGTGAAGTCTTTTAAACTTTGTAAGAACAGTGCCACGGGGAAGACAT
GTGAAGTTGACTCCACCCCAGTGAAATGCCGACAAGGATTTTGTCTGAAGATTACACAAGAAGG
TCGAGGCCACGTCAAACTTTCAAGGGGAAGTGAAGTCGTTTTGGACGCTTGCGATTCATCCTGC
GAAGTAATGATTCCAAAAGGTACTGGAGACATTTTGGTGGATTGTTCAGGCGGGCAACAACACT
TTCTGAAAGACAACCTGATAGACCTCGGATGCCCTCATATCCCCCTGCTTGGTAGGATGGCAAT
CTATATCTGTCGGATGTCAAATCATCCTCGAACTACGATGGCATTCCTCTTTTGGTTCTCTTTT
GGTTACGTCATTACATGCATTTTCTGTAAGGCACTCTTTTATTCACTCATTATCATCGGGACAC
TTGGAAAAAAAATCAAACAATACCGGGAACTTAAGCCCCAAACCTGCACCATTTGCGAAACTGC
GCCAGTAAACGCAATCGATGCCGAAATGCATGATCTCAATTGTTCATACAACATATGTCCCTAC
TGTGCGAGCAGGCTTACTAGTGATGGTTTGGCACGACACGTAACTCAGTGTCCTAAGAGAAAAG
AAAAAGTCGAAGAAACAGAATTGTACCTCAACCTCGAACGAATACCTTGGATCGTAAGGAAGCT
CCTG
SEQ ID NO: 6: CCHF Nucleoprotein (NP)
ATGGAAAACAAGATCGAGGTGAATAACAAAGATGAGATGAACAGGTGGTTTGAAGAGTTCAAAA
AAGGAAATGGACTTGTGGACACCTTCACAAACTCCTATTCCTTTTGCGAGAGTGTTCCCAATTT
GGACAGGTTTGTGTTTCAGATGGCCAGTGCCACCGATGATGCACAGAAGGACTCCATCTACGCA
TCTGCTCTGGTGGAGGCAACAAAGTTTTGTGCACCTATATATGAGTGCGCATGGGTTAGCTCCA
CTGGCATTGTAAAAAAGGGACTTGAATGGTTCGAGAAAAATGCAGGAACCATTAAGTCCTGGGA
TGAAAGTTATACTGAGCTAAAGGTCGACGTCCCGAAAATAGAGCAGCTTACCGGTTACCAACAA
GCTGCCTTGAAGTGGAGAAAAGACATAGGTTTCCGTGTCAATGCCAACACAGCAGCTCTGAGCA
ACAAAGTCCTCGCAGAATACAAAGTCCCTGGTGAGATTGTGATGTCTGTCAAAGAGATGCTGTC
AGACATGATTAGGAGAAGGAACCTGATTCTAAACAGGGGTGGTGATGAGAACCCACGTGGCCCA
GTGAGCCATGAGCATGTAGACTGGTGCAGGGAGTTTGTCAAAGGCAAATACATCATGGCCTTCA
ACCCACCATGGGGGGACATCAACAAGTCAGGCCGTTCAGGAATAGCACTTGTTGCAACAGGCCT
TGCTAAGCTTGCAGAGACTGAAGGAAAGGGAATATTTGATGAAGCCAAAAAGACTGTGGAGGCC
CTCAACGGGTATCTGGACAAGCATAAGGACGAAGTTGATAGAGCAAGCGCCGACAGCATGATAA
CAAACCTTCTTAAGCATATTGCCAAGGCACAGGAGCTCTATAAAAATTCATCTGCACTTCGTGC
ACAAAGCGCACAGATTGACACTGCTTTCAGCTCATACTATTGGCTTTACAAGGCTGGCGTGACT
CCTGAAACCTTCCCGACGGTGTCACAGTTCCTCTTTGAGCTAGGGAAACAGCCAAGAGGTACCA
AGAAAATGAAGAAGGCTCTTCTGAGCACCCCAATGAAGTGGGGGAAGAAGCTTTATGAGCTCTT
TGCCGATGATTCTTTCCAGCAGAACAGGATTTACATGCATCCTGCCGTGCTTACAGCTGGTAGA
ATCAGTGAAATGGGAGTCTGCTTTGGGACAATCCCTGTGGCCAATCCTGATGATGCTGCCCAAG
GATCTGGACACACTAAGTCTATTCTCAACCTCCGTACCAACACTGAGACCAATAATCCGTGTGC
CAAAACCATCGTCAAGCTATTTGAAGTTCAAAAAACAGGGTTCAACATTCAGGACATGGACATA
GTGGCCTCTGAGCACTTGCTACACCAATCCCTTGTTGGCAAGCAATCCCCATTCCAGAACGCCT
ACAACGTCAAGGGCAATGCCACCAGTGCTAACATCATTTAA
SEQ ID NO: 7: Crimean Congo Hemorrhagic Fever Virus glycoprotein
precursor (CCHF GP-Turkey-kk06) amino acid sequence
MPTNIMHTPLVCFILYLQLLCLGGAHGQLNATEHNGTNNTTAPGASQSPKPPMSTTPPHAPESS
TIKPTTPISEAEGSGETTSPPNTTQGLSSPETTSERPATTSISTSSTDSTNPTTQMTDNTPTPT
VSTSPSSSPSTPSTPQGIHHPARSLLSVSSPKTVTTPTPTSPGEMSSETSSQHSAMSRIPTPHT
ATRVSTEITNHRTPRQSESSAQQTTPSPMTSPAQSILLMSAAPTAVQDIHPSPTNRSKRNLETE
IILTLSQGLKKYYGKILKLLHLTLEQDTEGLLEWCKGNLGSNCDDDFFQKRIEEFFMTGECYFN
EVLQFKTLSTLSPTEPSHARLPTAEPFKSYFAKGFLSIDSGYFSAKCYPRSSASGLQLINVTQH
PARIAETPGPKTTSLKTINCINLRASVFKEHREVEINVLLPQIAVNLSNCHVVINSHVCDYSLD
TDGPVRLPRIYHEGTFIPGTYKIVIDKKNKLNDRCTLVTNCVIKGREVRKGQSVLRQYKTEIKI
GKASTGFRKLLSEEPGDDCISRTQLLRTETAEIHDDNYGGPGDKITICNGSTIVDQRLGSELGC
YTINRVKSFKLCKNSATGKTCEVDSTPVKCRQGFCLKITQEGRGHVKLSRGSEVVLDACDSSCE
VMIPKGTGDILVDCSGGQQHFLKDNLIDLGCPHIPLLGRMAIYICRMSNHPRTTMAFLFWFSFG
YVITCIFCKALFYSLIIIGTLGKKIKQYRELKPQTCTICETAPVNAIDAEMHDLNCSYNICPYC
ASRLTSDGLARHVTQCPKRKEKVEETELYLNLERIPWIVRKLLQVSESTGVALKRSSWLIVLLV
LLTVSLSPVQSAPVGHGKTIEIYQTREGFASICLFMLGSILFIVSCLVKGLVDSVSESFFPGLS
VCKTCSIGSVNGFEIESHKCYCSLFCCPYCRHCSADREIHQLHLSICKKRKTGSNVMLAVCKRM
CFRATIEASRRALLIRSIINTTFVICILTLTICVVSTSAVEMENLPAGTWEREEDLTNFCHQEC
QVTETECLCPYEALVLRKPLFLDSIVKGMKNLLNSTSLETSLSIEAPWGAINVQSTFKPTVSTA
NIALSWSSVVHRGNKILVTGRSESIMKLEERTGVSWDLGVEDASESKLLTVSIMDLSQMYSPVF
EYLSGDRQVEEWPKATCTGDCPERCGCTSSTCLHKEWSHSRNWRCNPTWCWGVGTGCTCCGVDV
KDLFTDHMFVKWKVEYIKTEAIVCVALTSQERQCSLIEAGTRFNLGPVTITLSEPRNIQQKLPP
ElITLHPKIEEGFFDLMHVQKVLSASTVCKLQSCTHGIPGDLQVYHIGNLLKGDRVNGHLIHKI
ESHFNTSWMSWDGCDLDYYCNMGDWPSCTYTGVTQHNHAAFVNLLNIETDYTKTFHFHSKRVTA
HGDTPQLDLKARPTYGAGGITVLVEVADMELHTKKVEISGLKFASLACTGCYACSSGISCKVRI
HVDEPDELTVHVKSSDPDVVAASTSLMARKLEFGTDSTFKAFSAMPKTSLCFYIVEREYCKSCS
EDDTQKCVDTRLEQPQSILIEHKGTIIGKQNDTCTAKASCWLESVKSFFYGLKNMLGSVFGNLF
IGILLFLAPFVLLVLFFMFGWKILFCFKCCRRTRGLFKYRHLKDDEETGYRRIIERLNSKKGKN
RLLDGDRLADRKIAELFSTKTHIG
SEQ ID NO: 8: Ubiquitin-CCHF Glycoprotein GC (the ubiquitin-related
sequence is underlined)-amino acid sequence
MQIFVKTLTGKTITLEVEPSDTIENVKAKIQDKEGIPPDQQRLIFAGKQLEDGRTLSDYNIQKE
STLHLVLRLRGGFLDSIVKGMKNLLNSTSLETSLSIEAPWGAINVQSTFKPTVSTANIALSWSS
VVHRGNKILVTGRSESIMKLEERTGVSWDLGVEDASESKLLTVSIMDLSQMYSPVFEYLSGDRQ
VEEWPKATCTGDCPERCGCTSSTCLHKEWSHSRNWRCNPTWCWGVGTGCTCCGVDVKDLFTDHM
FVKWKVEYIKTEAIVCVALTSQERQCSLIEAGTRFNLGPVTITLSEPRNIQQKLPPEIITLHPK
IEEGFFDLMHVQKVLSASTVCKLQSCTHGIPGDLQVYHIGNLLKGDRVNGHLIHKIESHFNTSW
MSWDGCDLDYYCNMGDWPSCTYTGVTQHNHAAFVNLLNIETDYTKTFHFHSKRVTAHGDTPQLD
LKARPTYGAGGITVLVEVADMELHTKKVEISGLKFASLACTGCYACSSGISCKVRIHVDEPDEL
TVHVKSSDPDVVAASTSLMARKLEFGTDSTFKAFSAMPKTSLCFYIVEREYCKSCSEDDTQKCV
DTRLEQPQSILIEHKGTIIGKQNDTCTAKASCWLESVKSFFYGLKNMLGSVFGNLFIGILLFLA
PFVLLVLFFMFGWKILFCFKCCRRTRGLFKYRHLKDDEETGYRRIIERLNSKKGKNRLLDGDRL
ADRKIAELFSTKTHIG
SEQ ID NO: 9: Ubiquitin-CCHF Glycoprotein Gn (the ubiquitin-related
sequence is underlined)-amino acid sequence
MQIFVKTLTGKTITLEVEPSDTIENVKAKIQDKEGIPPDQQRLIFAGKQLEDGRTLSDYNIQKE
STLHLVLRLRGGSEEPGDDCISRTQLLRTETAEIHDDNYGGPGDKITICNGSTIVDQRLGSELG
CYTINRVKSFKLCKNSATGKTCEVDSTPVKCRQGFCLKITQEGRGHVKLSRGSEVVLDACDSSC
EVMIPKGTGDILVDCSGGQQHFLKDNLIDLGCPHIPLLGRMAIYICRMSNHPRTTMAFLFWFSF
GYVITCIFCKALFYSLIIIGTLGKKIKQYRELKPQTCTICETAPVNAIDAEMHDLNCSYNICPY
CASRLTSDGLARHVTQCPKRKEKVEETELYLNLERIPWIVRKLL
SEQ ID NO: 10: CCHF Nucleoprotein (NP)-amino acid sequence
MENKIEVNNKDEMNRWFEEFKKGNGLVDTFTNSYSFCESVPNLDRFVFQMASATDDAQKDSIYA
SALVEATKFCAPIYECAWVSSTGIVKKGLEWFEKNAGTIKSWDESYTELKVDVPKIEQLTGYQQ
AALKWRKDIGFRVNANTAALSNKVLAEYKVPGEIVMSVKEMLSDMIRRRNLILNRGGDENPRGP
VSHEHVDWCREFVKGKYIMAFNPPWGDINKSGRSGIALVATGLAKLAETEGKGIFDEAKKTVEA
LNGYLDKHKDEVDRASADSMITNLLKHIAKAQELYKNSSALRAQSAQIDTAFSSYYWLYKAGVT
PETFPTVSQFLFELGKQPRGTKKMKKALLSTPMKWGKKLYELFADDSFQQNRIYMHPAVLTAGR
ISEMGVCFGTIPVANPDDAAQGSGHTKSILNLRTNTETNNPCAKTIVKLFEVQKTGFNIQDMDI
VASEHLLHQSLVGKQSPFQNAYNVKGNATSANII
SEQ ID NO: 11: pIDV-I plasmid
AGATCTTTTTTCCCTCTGCCAAAAATTATGGGGACATCATGAAGCCCCTTGAGCATCTGACTTC
TGGCTAATAAAGGAAATTTATTTTCATTGCAATAGTGTGTTGGAATTTTTTGTGTCTCTCACTC
GGAAGGACATATGGGAGGGCAAATCATTTAAAACATCAGAATGAGTATTTGGTTTAGAGTTTGG
CAACATATGCCCATATGCTGGCTGCCATGAACAAAGGTTGGCTATAAAGAGGTCATCAGTATAT
GAAACAGCCCCCTGCTGTCCATTCCTTATTCCATAGAAAAGCCTTGACTTGAGGTTAGATTTTT
TTTATATTTTGTTTTGTGTTATTTTTTTCTTTAACATCCCTAAAATTTTCCTTACATGTTTTAC
TAGCCAGATTTTTCCTCCTCTCCTGACTACTCCCAGTCATAGCTGTCCCTCTTCTCTTATGGAG
ATCCCTCGACCTGCAGCCCAAgctTGTTGCTGGCGTTTTTCCATAGGCTCCGCCCCCCTGACGA
GCATCACAAAAATCGACGCTCAAGTCAGAGGTGGCGAAACCCGACAGGACTATAAAGATACCAG
GCGTTTCCCCCTGGAAGCTCCCTCGTGCGCTCTCCTGTTCCGACCCTGCCGCTTACCGGATACC
TGTCCGCCTTTCTCCCTTCGGGAAGCGTGGCGCTTTCTCATAGCTCACGCTGTAGGTATCTCAG
TTCGGTGTAGGTCGTTCGCTCCAAGCTGGGCTGTGTGCACGAACCCCCCGTTCAGCCCGACCGC
TGCGCCTTATCCGGTAACTATCGTCTTGAGTCCAACCCGGTAAGACACGACTTATCGCCACTGG
CAGCAGCCACTGGTAACAGGATTAGCAGAGCGAGGTATGTAGGCGGTGCTACAGAGTTCTTGAA
GTGGTGGCCTAACTACGGCTACACTAGAAGAACAGTATTTGGTATCTGCGCTCTGCTGAAGCCA
GTTACCTTCGGAAAAAGAGTTGGTAGCTCTTGATCCGGCAAACAAACCACCGCTGGTAGCGGTG
GTTTTTTTGTTTGCAAGCAGCAGATTACGCGCAGAAAAAAAGGATCTCAAGAAGATCCTTTGAT
CTGTCTGACGCTCAGTGGAACGAAAACTCACGTTAAGGGATTTTGGTCATGAGATTATCAAAAA
GGATCTTCACCTAGATCCTTTTAAATTAAAAATGAAGTTTTAGCACGTGCTATTATTGAAGCAC
ACATTTCCCCGAAAAGTGCCACCTGTATGCGGTGTGAAATACCGCACAGATGCGTAAGGAGAAA
ATACCGCATCAGGAAATTGTAAGCGTTAATAATTCAGAAGAACTCGTCAAGAAGGCGATAGAAG
GCGATGCGCTGCGAATCGGGAGCGGCGATACCGTAAAGCACGAGGAAGCGGTCAGCCCATTCGC
CGCCAAGCTCTTCAGCAATATCACGGGTAGCCAACGCTATGTCCTGATAGCGGTCCGCCACACC
CAGCCGGCCACAGTCGATGAATCCAGAAAAGCGGCCATTTTCCACCATGATATTCGGCAAGCAG
GCATCGCCATGGGTCACGACGAGATCCTCGCCGTCGGGCATGCTCGCCTTGAGCCTGGCGAACA
GTTCGGCTGGCGCGAGCCCCTGATGCTCTTCGTCCAGATCATCCTGATCGACAAGACCGGCTTC
CATCCGAGTACGTGCTCGCTCGATGCGATGTTTCGCTTGGTGGTCGAATGGGCAGGTAGCCGGA
TCAAGCGTATGCAGCCGCCGCATTGCATCAGCCATGATGGATACTTTCTCGGCAGGAGCAAGGT
GAGATGACAGGAGATCCTGCCCCGGCACTTCGCCCAATAGCAGCCAGTCCCTTCCCGCTTCAGT
GACAACGTCGAGCACAGCTGCGCAAGGAACGCCCGTCGTGGCCAGCCACGATAGCCGCGCTGCC
TCGTCTTGCAGTTCATTCAGGGCACCGGACAGGTCGGTCTTGACAAAAAGAACCGGGCGCCCCT
GCGCTGACAGCCGGAACACGGCGGCATCAGAGCAGCCGATTGTCTGTTGTGCCCAGTCATAGCC
GAATAGCCTCTCCACCCAAGCGGCCGGAGAACCTGCGTGCAATCCATCTTGTTCAATCATGCGA
AACGATCCTCATCCTGTCTCTTGATCAGAGCTTGATCCCCTGCGCCATCAGATCCTTGGCGGCG
AGAAAGCCATCCAGTTTACTTTGCAGGGCTTCCCAACCTTACCAGAGGGCGCCCCAGCTGGCAA
TTCCGGTTCGCTTGCTGTCCATAAAACCGCCCAGTAGAAGGCATGCCTGCTACTAGTTATTAAT
AGTAATCAATTACGGGGTCATTAGTTCATAGCCCATATATGGAGTTCCGCGTTACATAACTTAC
GGTAAATGGCCCGCCTGGCTGACCGCCCAACGACCCCCGCCCATTGACGTCAATAATGACGTAT
GTTCCCATAGTAACGCCAATAGGGACTTTCCATTGACGTCAATGGGTGGAGTATTTACGGTAAA
CTGCCCACTTGGCAGTACATCAAGTGTATCATATGCCAAGTACGCCCCCTATTGACGTCAATGA
CGGTAAATGGCCCGCCTGGCATTATGCCCAGTACATGACCTTATGGGACTTTCCTACTTGGCAG
TACATCTACGTATTAGTCATCGCTATTACCATGGTCGAGGTGAGCCCCACGTTCTGCTTCACTC
TCCCCATCTCCCCCCCCTCCCCACCCCCAATTTTGTATTTATTTATTTTTTAATTATTTTGTGC
AGCGATGGGGGCGGGGGGGGGGGGGGGGCGCGCGCCAGGCGGGGCGGGGCGGGGCGAGGGGCGG
GGCGGGGCGAGGCGGAGAGGTGCGGCGGCAGCCAATCAGAGCGGCGCGCTCCGAAAGTTTCCTT
TTATGGCGAGGCGGCGGCGGCGGCGGCCCTATAAAAAGCGAAGCGCGCGGCGGGCGGGAGTCGC
TGCGCGCTGCCTTCGCCCCGTGCCCCGCTCCGCCGCCGCCTCGCGCCGCCCGCCCCGGCTCTGA
CTGACCGCGTTACTCCCACAGGTGAGCGGGCGGGACGGCCCTTCTCCTCCGGGCTGTAATTAGC
GCTTGGTTTAATGACGGCTCGTTTCTTTTCTGTGGCTGCGTGAAAGCCTTAAAGGGCTCCGGGA
GGGCCCTTTGTGCGGGGGGAGCGGCTCGGGGGGTGCGTGCGTGTGTGTGTGCGTGGGGAGCGCC
GCGTGCGGCTCCGCGCTGCCCGGCGGCTGTGAGCGCTGCGGGCGCGGCGCGGGGCTTTGTGCGC
TCCGCAGTGTGCGCGAGGGGAGCGCGGCCGGGGGCGGTGCCCCGCGGTGCGGGGGGGGCTGCGA
GGGGAACAAAGGCTGCGTGCGGGGTGTGTGCGTGGGGGGGTGAGCAGGGGGTGTGGGCGCGTCG
GTCGGGCTGCAACCCCCCCTGCACCCCCCTCCCCGAGTTGCTGAGCACGGCCCGGCTTCGGGTG
CGGGGCTCCGTACGGGGCGTGGCGCGGGGCTCGCCGTGCCGGGCGGGGGGTGGCGGCAGGTGGG
GGTGCCGGGCGGGGCGGGGCCGCCTCGGGCCGGGGAGGGCTCGGGGGAGGGGCGCGGCGGCCCC
CGGAGCGCCGGCGGCTGTCGAGGCGCGGCGAGCCGCAGCCATTGCCTTTTATGGTAATCGTGCG
AGAGGGCGCAGGGACTTCCTTTGTCCCAAATCTGTGCGGAGCCGAAATCTGGGAGGCGCCGCCG
CACCCCCTCTAGCGGGCGCGGGGCGAAGCGGTGCGGCGCCGGCAGGAAGGAAATGGGCGGGGAG
GGCCTTCGTGCGTCGCCGCGCCGCCGTCCCCTTCTCCCTCTCCAGCCTCGGGGCTGTCCGCGGG
GGGACGGCTGCCTTCGGGGGGGACGGGGCAGGGCGGGGTTCGGCTTCTGGCGTGTGACCGGCGG
CTCTAGAGCCTCTGCTAACCATGTTCATGCCTTCTTCTTTTTCCTACAGCTCCTGGGCAACGTG
CTGGTTATTGTGCTGTCTCATCATTTTGGCAAAGAATTCGAGCTCATCGATGCATGGTACC
SEQ ID NO: 12: pIDV-II (WPRE position 7-595)
AGATCTaatcaacctctggattacaaaatttgtgaaagattgactggtattcttaactatgttg
ctccttttacgctatgtggatacgctgctttaatgcctttgtatcatgctattgcttcccgtat
ggctttcattttctcctccttgtataaatcctggttgctgtctctttatgaggagttgtggccc
gttgtcaggcaacgtggcgtggtgtgcactgtgtttgctgacgcaacccccactggttggggca
ttgccaccacctgtcagctcctttccgggactttcgctttccccctccctattgccacggcgga
actcatcgccgcctgccttgcccgctgctggacaggggctcggctgttgggcactgacaattcc
gtggtgttgtcggggaagctgacgtcctttccatggctgctcgcctgtgttgccacctggattc
tgcgcgggacgtccttctgctacgtcccttcggccctcaatccagcggaccttccttcccgcgg
cctgctgccggctctgcggcctcttccgcgtcttcgccttcgccctcagacgagtcggatctcc
ctttgggccgcctccccgcTTTTTCCCTCTGCCAAAAATTATGGGGACATCATGAAGCCCCTTG
AGCATCTGACTTCTGGCTAATAAAGGAAATTTATTTTCATTGCAATAGTGTGTTGGAATTTTTT
GTGTCTCTCACTCGGAAGGACATATGGGAGGGCAAATCATTTAAAACATCAGAATGAGTATTTG
GTTTAGAGTTTGGCAACATATGCCCATATGCTGGCTGCCATGAACAAAGGTTGGCTATAAAGAG
GTCATCAGTATATGAAACAGCCCCCTGCTGTCCATTCCTTATTCCATAGAAAAGCCTTGACTTG
AGGTTAGATTTTTTTTATATTTTGTTTTGTGTTATTTTTTTCTTTAACATCCCTAAAATTTTCC
TTACATGTTTTACTAGCCAGATTTTTCCTCCTCTCCTGACTACTCCCAGTCATAGCTGTCCCTC
TTCTCTTATGGAGATCCCTCGACCTGCAGCCCAAgctTGTTGCTGGCGTTTTTCCATAGGCTCC
GCCCCCCTGACGAGCATCACAAAAATCGACGCTCAAGTCAGAGGTGGCGAAACCCGACAGGACT
ATAAAGATACCAGGCGTTTCCCCCTGGAAGCTCCCTCGTGCGCTCTCCTGTTCCGACCCTGCCG
CTTACCGGATACCTGTCCGCCTTTCTCCCTTCGGGAAGCGTGGCGCTTTCTCATAGCTCACGCT
GTAGGTATCTCAGTTCGGTGTAGGTCGTTCGCTCCAAGCTGGGCTGTGTGCACGAACCCCCCGT
TCAGCCCGACCGCTGCGCCTTATCCGGTAACTATCGTCTTGAGTCCAACCCGGTAAGACACGAC
TTATCGCCACTGGCAGCAGCCACTGGTAACAGGATTAGCAGAGCGAGGTATGTAGGCGGTGCTA
CAGAGTTCTTGAAGTGGTGGCCTAACTACGGCTACACTAGAAGAACAGTATTTGGTATCTGCGC
TCTGCTGAAGCCAGTTACCTTCGGAAAAAGAGTTGGTAGCTCTTGATCCGGCAAACAAACCACC
GCTGGTAGCGGTGGTTTTTTTGTTTGCAAGCAGCAGATTACGCGCAGAAAAAAAGGATCTCAAG
AAGATCCTTTGATCTGTCTGACGCTCAGTGGAACGAAAACTCACGTTAAGGGATTTTGGTCATG
AGATTATCAAAAAGGATCTTCACCTAGATCCTTTTAAATTAAAAATGAAGTTTTAGCACGTGCT
ATTATTGAAGCACACATTTCCCCGAAAAGTGCCACCTGTATGCGGTGTGAAATACCGCACAGAT
GCGTAAGGAGAAAATACCGCATCAGGAAATTGTAAGCGTTAATAATTCAGAAGAACTCGTCAAG
AAGGCGATAGAAGGCGATGCGCTGCGAATCGGGAGCGGCGATACCGTAAAGCACGAGGAAGCGG
TCAGCCCATTCGCCGCCAAGCTCTTCAGCAATATCACGGGTAGCCAACGCTATGTCCTGATAGC
GGTCCGCCACACCCAGCCGGCCACAGTCGATGAATCCAGAAAAGCGGCCATTTTCCACCATGAT
ATTCGGCAAGCAGGCATCGCCATGGGTCACGACGAGATCCTCGCCGTCGGGCATGCTCGCCTTG
AGCCTGGCGAACAGTTCGGCTGGCGCGAGCCCCTGATGCTCTTCGTCCAGATCATCCTGATCGA
CAAGACCGGCTTCCATCCGAGTACGTGCTCGCTCGATGCGATGTTTCGCTTGGTGGTCGAATGG
GCAGGTAGCCGGATCAAGCGTATGCAGCCGCCGCATTGCATCAGCCATGATGGATACTTTCTCG
GCAGGAGCAAGGTGAGATGACAGGAGATCCTGCCCCGGCACTTCGCCCAATAGCAGCCAGTCCC
TTCCCGCTTCAGTGACAACGTCGAGCACAGCTGCGCAAGGAACGCCCGTCGTGGCCAGCCACGA
TAGCCGCGCTGCCTCGTCTTGCAGTTCATTCAGGGCACCGGACAGGTCGGTCTTGACAAAAAGA
ACCGGGCGCCCCTGCGCTGACAGCCGGAACACGGCGGCATCAGAGCAGCCGATTGTCTGTTGTG
CCCAGTCATAGCCGAATAGCCTCTCCACCCAAGCGGCCGGAGAACCTGCGTGCAATCCATCTTG
TTCAATCATGCGAAACGATCCTCATCCTGTCTCTTGATCAGAGCTTGATCCCCTGCGCCATCAG
ATCCTTGGCGGCGAGAAAGCCATCCAGTTTACTTTGCAGGGCTTCCCAACCTTACCAGAGGGCG
CCCCAGCTGGCAATTCCGGTTCGCTTGCTGTCCATAAAACCGCCCAGTAGAAGGCATGCCTGCT
ACTAGTTATTAATAGTAATCAATTACGGGGTCATTAGTTCATAGCCCATATATGGAGTTCCGCG
TTACATAACTTACGGTAAATGGCCCGCCTGGCTGACCGCCCAACGACCCCCGCCCATTGACGTC
AATAATGACGTATGTTCCCATAGTAACGCCAATAGGGACTTTCCATTGACGTCAATGGGTGGAG
TATTTACGGTAAACTGCCCACTTGGCAGTACATCAAGTGTATCATATGCCAAGTACGCCCCCTA
TTGACGTCAATGACGGTAAATGGCCCGCCTGGCATTATGCCCAGTACATGACCTTATGGGACTT
TCCTACTTGGCAGTACATCTACGTATTAGTCATCGCTATTACCATGGTCGAGGTGAGCCCCACG
TTCTGCTTCACTCTCCCCATCTCCCCCCCCTCCCCACCCCCAATTTTGTATTTATTTATTTTTT
AATTATTTTGTGCAGCGATGGGGGCGGGGGGGGGGGGGGGGCGCGCGCCAGGCGGGGCGGGGCG
GGGCGAGGGGCGGGGCGGGGCGAGGCGGAGAGGTGCGGCGGCAGCCAATCAGAGCGGCGCGCTC
CGAAAGTTTCCTTTTATGGCGAGGCGGCGGCGGCGGCGGCCCTATAAAAAGCGAAGCGCGCGGC
GGGCGGGAGTCGCTGCGCGCTGCCTTCGCCCCGTGCCCCGCTCCGCCGCCGCCTCGCGCCGCCC
GCCCCGGCTCTGACTGACCGCGTTACTCCCACAGGTGAGCGGGCGGGACGGCCCTTCTCCTCCG
GGCTGTAATTAGCGCTTGGTTTAATGACGGCTCGTTTCTTTTCTGTGGCTGCGTGAAAGCCTTA
AAGGGCTCCGGGAGGGCCCTTTGTGCGGGGGGAGCGGCTCGGGGGGTGCGTGCGTGTGTGTGTG
CGTGGGGAGCGCCGCGTGCGGCTCCGCGCTGCCCGGCGGCTGTGAGCGCTGCGGGCGCGGCGCG
GGGCTTTGTGCGCTCCGCAGTGTGCGCGAGGGGAGCGCGGCCGGGGGCGGTGCCCCGCGGTGCG
GGGGGGGCTGCGAGGGGAACAAAGGCTGCGTGCGGGGTGTGTGCGTGGGGGGGTGAGCAGGGGG
TGTGGGCGCGTCGGTCGGGCTGCAACCCCCCCTGCACCCCCCTCCCCGAGTTGCTGAGCACGGC
CCGGCTTCGGGTGCGGGGCTCCGTACGGGGCGTGGCGCGGGGCTCGCCGTGCCGGGCGGGGGGT
GGCGGCAGGTGGGGGTGCCGGGCGGGGCGGGGCCGCCTCGGGCCGGGGAGGGCTCGGGGGAGGG
GCGCGGCGGCCCCCGGAGCGCCGGCGGCTGTCGAGGCGCGGCGAGCCGCAGCCATTGCCTTTTA
TGGTAATCGTGCGAGAGGGCGCAGGGACTTCCTTTGTCCCAAATCTGTGCGGAGCCGAAATCTG
GGAGGCGCCGCCGCACCCCCTCTAGCGGGCGCGGGGCGAAGCGGTGCGGCGCCGGCAGGAAGGA
AATGGGCGGGGAGGGCCTTCGTGCGTCGCCGCGCCGCCGTCCCCTTCTCCCTCTCCAGCCTCGG
GGCTGTCCGCGGGGGGACGGCTGCCTTCGGGGGGGACGGGGCAGGGCGGGGTTCGGCTTCTGGC
GTGTGACCGGCGGCTCTAGAGCCTCTGCTAACCATGTTCATGCCTTCTTCTTTTTCCTACAGCT
CCTGGGCAACGTGCTGGTTATTGTGCTGTCTCATCATTTTGGCAAAGAATTCGAGCTCATCGAT
GCATGGTACC
SEQ ID NO: 13: pIDV-II-CCHF-GP-Turkey (CCFH Turkey antigen 4613-9688)
Taatcaacctctggattacaaaatttgtgaaagattgactggtattcttaactatgttgctcct
tttacgctatgtggatacgctgctttaatgcctttgtatcatgctattgcttcccgtatggctt
tcattttctcctccttgtataaatcctggttgctgtctctttatgaggagttgtggcccgttgt
caggcaacgtggcgtggtgtgcactgtgtttgctgacgcaacccccactggttggggcattgcc
accacctgtcagctcctttccgggactttcgctttccccctccctattgccacggcggaactca
tcgccgcctgccttgcccgctgctggacaggggctcggctgttgggcactgacaattccgtggt
gttgtcggggaagctgacgtcctttccatggctgctcgcctgtgttgccacctggattctgcgc
gggacgtccttctgctacgtcccttcggccctcaatccagcggaccttccttcccgcggcctgc
tgccggctctgcggcctcttccgcgtcttcgccttcgccctcagacgagtcggatctccctttg
ggccgcctccccgcTTTTTCCCTCTGCCAAAAATTATGGGGACATCATGAAGCCCCTTGAGCAT
CTGACTTCTGGCTAATAAAGGAAATTTATTTTCATTGCAATAGTGTGTTGGAATTTTTTGTGTC
TCTCACTCGGAAGGACATATGGGAGGGCAAATCATTTAAAACATCAGAATGAGTATTTGGTTTA
GAGTTTGGCAACATATGCCCATATGCTGGCTGCCATGAACAAAGGTTGGCTATAAAGAGGTCAT
CAGTATATGAAACAGCCCCCTGCTGTCCATTCCTTATTCCATAGAAAAGCCTTGACTTGAGGTT
AGATTTTTTTTATATTTTGTTTTGTGTTATTTTTTTCTTTAACATCCCTAAAATTTTCCTTACA
TGTTTTACTAGCCAGATTTTTCCTCCTCTCCTGACTACTCCCAGTCATAGCTGTCCCTCTTCTC
TTATGGAGATCCCTCGACCTGCAGCCCAAgctTGTTGCTGGCGTTTTTCCATAGGCTCCGCCCC
CCTGACGAGCATCACAAAAATCGACGCTCAAGTCAGAGGTGGCGAAACCCGACAGGACTATAAA
GATACCAGGCGTTTCCCCCTGGAAGCTCCCTCGTGCGCTCTCCTGTTCCGACCCTGCCGCTTAC
CGGATACCTGTCCGCCTTTCTCCCTTCGGGAAGCGTGGCGCTTTCTCATAGCTCACGCTGTAGG
TATCTCAGTTCGGTGTAGGTCGTTCGCTCCAAGCTGGGCTGTGTGCACGAACCCCCCGTTCAGC
CCGACCGCTGCGCCTTATCCGGTAACTATCGTCTTGAGTCCAACCCGGTAAGACACGACTTATC
GCCACTGGCAGCAGCCACTGGTAACAGGATTAGCAGAGCGAGGTATGTAGGCGGTGCTACAGAG
TTCTTGAAGTGGTGGCCTAACTACGGCTACACTAGAAGAACAGTATTTGGTATCTGCGCTCTGC
TGAAGCCAGTTACCTTCGGAAAAAGAGTTGGTAGCTCTTGATCCGGCAAACAAACCACCGCTGG
TAGCGGTGGTTTTTTTGTTTGCAAGCAGCAGATTACGCGCAGAAAAAAAGGATCTCAAGAAGAT
CCTTTGATCTGTCTGACGCTCAGTGGAACGAAAACTCACGTTAAGGGATTTTGGTCATGAGATT
ATCAAAAAGGATCTTCACCTAGATCCTTTTAAATTAAAAATGAAGTTTTAGCACGTGCTATTAT
TGAAGCACACATTTCCCCGAAAAGTGCCACCTGTATGCGGTGTGAAATACCGCACAGATGCGTA
AGGAGAAAATACCGCATCAGGAAATTGTAAGCGTTAATAATTCAGAAGAACTCGTCAAGAAGGC
GATAGAAGGCGATGCGCTGCGAATCGGGAGCGGCGATACCGTAAAGCACGAGGAAGCGGTCAGC
CCATTCGCCGCCAAGCTCTTCAGCAATATCACGGGTAGCCAACGCTATGTCCTGATAGCGGTCC
GCCACACCCAGCCGGCCACAGTCGATGAATCCAGAAAAGCGGCCATTTTCCACCATGATATTCG
GCAAGCAGGCATCGCCATGGGTCACGACGAGATCCTCGCCGTCGGGCATGCTCGCCTTGAGCCT
GGCGAACAGTTCGGCTGGCGCGAGCCCCTGATGCTCTTCGTCCAGATCATCCTGATCGACAAGA
CCGGCTTCCATCCGAGTACGTGCTCGCTCGATGCGATGTTTCGCTTGGTGGTCGAATGGGCAGG
TAGCCGGATCAAGCGTATGCAGCCGCCGCATTGCATCAGCCATGATGGATACTTTCTCGGCAGG
AGCAAGGTGAGATGACAGGAGATCCTGCCCCGGCACTTCGCCCAATAGCAGCCAGTCCCTTCCC
GCTTCAGTGACAACGTCGAGCACAGCTGCGCAAGGAACGCCCGTCGTGGCCAGCCACGATAGCC
GCGCTGCCTCGTCTTGCAGTTCATTCAGGGCACCGGACAGGTCGGTCTTGACAAAAAGAACCGG
GCGCCCCTGCGCTGACAGCCGGAACACGGCGGCATCAGAGCAGCCGATTGTCTGTTGTGCCCAG
TCATAGCCGAATAGCCTCTCCACCCAAGCGGCCGGAGAACCTGCGTGCAATCCATCTTGTTCAA
TCATGCGAAACGATCCTCATCCTGTCTCTTGATCAGAGCTTGATCCCCTGCGCCATCAGATCCT
TGGCGGCGAGAAAGCCATCCAGTTTACTTTGCAGGGCTTCCCAACCTTACCAGAGGGCGCCCCA
GCTGGCAATTCCGGTTCGCTTGCTGTCCATAAAACCGCCCAGTAGAAGGCATGCCTGCTACTAG
TTATTAATAGTAATCAATTACGGGGTCATTAGTTCATAGCCCATATATGGAGTTCCGCGTTACA
TAACTTACGGTAAATGGCCCGCCTGGCTGACCGCCCAACGACCCCCGCCCATTGACGTCAATAA
TGACGTATGTTCCCATAGTAACGCCAATAGGGACTTTCCATTGACGTCAATGGGTGGAGTATTT
ACGGTAAACTGCCCACTTGGCAGTACATCAAGTGTATCATATGCCAAGTACGCCCCCTATTGAC
GTCAATGACGGTAAATGGCCCGCCTGGCATTATGCCCAGTACATGACCTTATGGGACTTTCCTA
CTTGGCAGTACATCTACGTATTAGTCATCGCTATTACCATGGTCGAGGTGAGCCCCACGTTCTG
CTTCACTCTCCCCATCTCCCCCCCCTCCCCACCCCCAATTTTGTATTTATTTATTTTTTAATTA
TTTTGTGCAGCGATGGGGGCGGGGGGGGGGGGGGGGCGCGCGCCAGGCGGGGCGGGGCGGGGCG
AGGGGCGGGGCGGGGCGAGGCGGAGAGGTGCGGCGGCAGCCAATCAGAGCGGCGCGCTCCGAAA
GTTTCCTTTTATGGCGAGGCGGCGGCGGCGGCGGCCCTATAAAAAGCGAAGCGCGCGGCGGGCG
GGAGTCGCTGCGCGCTGCCTTCGCCCCGTGCCCCGCTCCGCCGCCGCCTCGCGCCGCCCGCCCC
GGCTCTGACTGACCGCGTTACTCCCACAGGTGAGCGGGCGGGACGGCCCTTCTCCTCCGGGCTG
TAATTAGCGCTTGGTTTAATGACGGCTCGTTTCTTTTCTGTGGCTGCGTGAAAGCCTTAAAGGG
CTCCGGGAGGGCCCTTTGTGCGGGGGGAGCGGCTCGGGGGGTGCGTGCGTGTGTGTGTGCGTGG
GGAGCGCCGCGTGCGGCTCCGCGCTGCCCGGCGGCTGTGAGCGCTGCGGGCGCGGCGCGGGGCT
TTGTGCGCTCCGCAGTGTGCGCGAGGGGAGCGCGGCCGGGGGCGGTGCCCCGCGGTGCGGGGGG
GGCTGCGAGGGGAACAAAGGCTGCGTGCGGGGTGTGTGCGTGGGGGGGTGAGCAGGGGGTGTGG
GCGCGTCGGTCGGGCTGCAACCCCCCCTGCACCCCCCTCCCCGAGTTGCTGAGCACGGCCCGGC
TTCGGGTGCGGGGCTCCGTACGGGGCGTGGCGCGGGGCTCGCCGTGCCGGGCGGGGGGTGGCGG
CAGGTGGGGGTGCCGGGCGGGGCGGGGCCGCCTCGGGCCGGGGAGGGCTCGGGGGAGGGGCGCG
GCGGCCCCCGGAGCGCCGGCGGCTGTCGAGGCGCGGCGAGCCGCAGCCATTGCCTTTTATGGTA
ATCGTGCGAGAGGGCGCAGGGACTTCCTTTGTCCCAAATCTGTGCGGAGCCGAAATCTGGGAGG
CGCCGCCGCACCCCCTCTAGCGGGCGCGGGGCGAAGCGGTGCGGCGCCGGCAGGAAGGAAATGG
GCGGGGAGGGCCTTCGTGCGTCGCCGCGCCGCCGTCCCCTTCTCCCTCTCCAGCCTCGGGGCTG
TCCGCGGGGGGACGGCTGCCTTCGGGGGGGACGGGGCAGGGCGGGGTTCGGCTTCTGGCGTGTG
ACCGGCGGCTCTAGAGCCTCTGCTAACCATGTTCATGCCTTCTTCTTTTTCCTACAGCTCCTGG
GCAACGTGCTGGTTATTGTGCTGTCTCATCATTTTGGCAAAGAATTCGAGCTCATCGATGCATG
GTACGGTACcgcaccATGCCAACTAACATCACCCACACCCTGCTGGTCTGCTTCATCCTGTATC
TGCAGCTGCTGGGGAGAGGCGGCGCACATGGACAGTCAAACGCCACAGAGCACAACGGCACCAA
TACCACAACCGCACCAGGCACCTCTCAGAGCCACAAGCCTCTGGTGAGCACAACCCCCCCTCAC
ACACTGGAGAGCTCCACCATCAAGCACACAACCCCCACCTCTGAGACAGAGGGAAGCGGAGAGA
CAACCCCACCACCTAACACAACCCAGGGACCTTCCCCACCAGAGGCAACCCCTGAGCGCCCAGC
AACAACCGCCACCAGCACACCCTCCACCGATAACACAAATAGCACAACCCAGATGAATGACAAC
AATCCTACCTCCACAATCTCCACATCTCCCTCTAGCTCCCCTTCTACCCCTCCAACACCTCAGG
GCATCCACCACCCAGCACGGAGCCTGCTGAGCGTGTCTAGCCTGAAGACCGCCACAACCCCAAC
CCCCACAAGCCCTGGCGAGATCAGCTCTGAGACAAGCTCCCAGCACTCCGCCATGTCTCGCACC
CCAACACTGCACACAACCACACAGGTGAGCACCGAGTCCACAAACCACTCCACCCCAAGGCAGT
CTGAGTCTAGCGCCCAGCCTACCACACCTTCCCCAATGACATCTCCAGCCCAGAGCATCCTGCC
CATGTCTGCCGCCCCTACCGCCATCCAGAATATCCACCCCAGCCCTACAAACCGGTCCAAGAGA
AATCTGGAGGTGGAGATCATCCTGACCCTGTCCCAGGGCCTGAAGAAGTACTATGGCAAGATCC
TGAAGCTGCTGCACCTGACACTGGAGGAGGATACCGAGGGCCTGCTGGAGTGGTGTAAGAGAAA
CCTGGGCTCCTCTTGCGACGATGACTTCTTTCAGAAGAGGATCGAGGAGTTCTTTGTGACCGGC
GAGGGCTACTTTAATGAGGTGCTGCAGTTCAAGACCCTGTCTACACTGAGCCCCACAGAGCCTA
GCCACGCCAAGCTGCCAACCGTGGAGCCCTTCAAGTCCTATTTTGCCAAGGGCTTCCTGTCCAT
CGACTCTGGCTACTTTTCCGCCAAGTGTTATCCACGCAGCTCCACATCTGGCCTGCAGCTGATC
AACGTGACCCAGCACCCAGCAAGGATCGCAGAGACACCAGGACCCAAGACCACATCTCTGAAGA
CCATCAACTGCATCAATCTGAGGGCCAGCGTGTTCAAGGAGCACCGCGAGATCGAGATCAATGT
GCTGCTGCCACAGATCGCCGTGAACCTGAGCAATTGTCACGCCGTGATCAAGTCTCACGTGTGC
GATTACAGCCTGGATACCGACGGCCCTGTGAGACTGCCACACATCTACCACGAGGGCACATTCA
TCCCCGGCACCTATAAGATCGTGATCGATAAGAAGAACAAGCTGAATGACAGGTGTATCCTGGT
GACCAACTGCGTGATCAAGGGAAGGGAGGTGCGCAAGGGACAGTCCGTGCTGAGACAGTATAAG
ACCGAGATCAAGATCGGCAAGGCCAGCACAGGCTCCAGGAAGCTGCTGTCCGAGGAGCCTGGCG
ATGACTGCATCTCTAGGACCCAGCTGCTGAGGACCGAGACAGCAGAGATCCACGATGACAACTA
CGGCGGCCCAGGCGATAAGATCACAATCTGTAATGGAAGCACCATCGTGGACCAGCGCCTGGGA
TCCGAGCTGGGCTGCTATACCATCAACCGGGTGAAGAGCTTTAAGCTGTGCGAGAATTCCGCCA
CCGGCAAGACATGCGAGATCGACAGCACCCCTGTGAAGTGTAGACAGGGCTTCTGCCTGAAGAT
CACACAGGAGGGCCGGGGCCACGTGAAGCTGTCTAGAGGCAGCGAGGTGGTGCTGGACGTGTGC
GACTCTAGCTGCGAAGTGATGATCCCAAAGGGCACCGGCGATATCCTGGTGGACTGCTCCGGAG
GACAGCAGCACTTTCTGAAGGATAACCTGATCGACCTGGGATGTCCACACGTGCCACTGCTGGG
AAGAATGGCCATCTACATCTGCCGGATGTCCAATCACCCCAGAACCACAATGGCCTTCCTGTTT
TGGTTCTCTTTTGGCTACGTGATCACCTGCATCTTTTGTAAGGCCCTGTTCTATAGCCTGATCA
TCATCGGCACACTGGGCAAGAAGTTCAAGCAGTATAGGGAGCTGAAGCCCCAGACCTGCACAAT
CTGTGAGACAGCCCCTGTGAACGCCATCGATGCCGAGATGCACGACCTGAACTGTTCCTACAAT
ATCTGCCCCTATTGTGCATCCAGGCTGACCTCTGATGGCCTGGCAAGACACGTGCCTCAGTGCC
CAAAGAGGAAGGAGAAGGTGGAGGAGACAGAGCTGTACCTGAATCTGGAGAGGATCCCTTGGAT
CGTGCGCAAGCTGCTGCAGGTGAGCGAGTCCACCGGAGTGGCCCTGAAGAGATCCTCTTGGCTG
ATCGTGCTGCTGGTGCTGCTGACAGTGTCTCTGAGCCCAGTGCAGAGCGCCCCAGTGGGACACG
GCAAGACCATCGAGACATATCAGACCAGGGAGGGCTTTACCTCCATCTGTCTGTTCATGCTGGG
CTCCATCCTGTTCATCGTGTCTTGCCTGGTGAAGGGCCTGGTGGATTCCGTGTCTGACAGCTTC
TTTCCCGGCCTGAGCGTGTGCAAGACCTGTTCCATCGGCTCTATCAACGGCTTTGAGATCGAGA
GCCACAAGTGCTACTGTTCCCTGTTCTGCTGTCCTTATTGCCGGCACTGTTCCGCCGACAGAGA
GATCCACCAGCTGCACCTGTCTATCTGCAAGAAGAGAAAGACCGGCAGCAACGTGATGCTGGCC
GTGTGCAAGAGGATGTGCTTTCGCGCCACAATCGAGGCCTCTCGGAGAGCCCTGCTGATCAGGA
GCATCATCAATACCACATTCGTGATCTGTATCCTGACCCTGACAATCTGCGTGGTGTCCACCTC
TGCCGTGGAGATGGAGAATCTGCCAGCAGGCACATGGGAGAGGGAGGAGGATCTGACCAACTTT
TGTCACCAGGAGTGCCAGGTGACCGAGACAGAGTGCCTGTGCCCATACGAGGCCCTGGTGCTGA
GGAAGCCTCTGTTCCTGGACAGCATCGTGAAGGGCATGAAGAACCTGCTGAATAGCACATCCCT
GGAGACAAGCCTGAGCATCGAGGCACCATGGGGAGCCATCAACGTGCAGTCTACCTTTAAGCCC
ACAGTGAGCACCGCCAATATCGCCCTGTCCTGGAGCTCCATCGAGCACCGCGGCAACAAGATCC
TGGTGACCGGCCGGTCCGAGTCTATCATGAAGCTGGAGGAGAGGACAGGCGTGAGCTGGGATCT
GGGAGTGGAGGACGCAAGCGAGTCCAAGCTGCTGACCGTGAGCATCATGGACCTGAGCCAGATG
TACTCCCCCGTGTTCGAGTATCTGTCCGGCGATAGACAGGTGGAGGAGTGGCCAAAGGCCACCT
GTACAGGCGACTGCCCCGAGAGGTGCGGCTGCACATCTAGCACCTGTCTGCACAAGGAGTGGCC
TCACAGCCGGAACTGGAGATGTAATCCAACCTGGTGCTGGGGAGTGGGCACAGGATGCACCTGC
TGTGGCGTGGATGTGAAGGACCTGTTTACAGATCACATGTTCGTGAAGTGGAAGGTGGAGTACA
TCAAGACCGAGGCCATCGTGTGCGTGGAGCTGACATCTCAGGAGAGACAGTGCAGCCTGATCGA
GGCCGGCACCAGGTTCAATCTGGGCCCAGTGACCATCACACTGAGCGAGCCCCGCAACATCCAG
CAGAAGCTGCCCCCTGAGATCATCACACTGCACCCAAAGGTGGAGGAGGGCTTCTTTGACCTGA
TGCACGTGCAGAAGGTGCTGTCTGCCAGCACCGTGTGCAAGCTGCAGTCCTGCACCCACGGAAT
CCCAGGCGATCTGCAGGTGTACCACATCGGCAACCTGCTGAAGGGCGACCGGGTGAATGGCCAC
CTGATCCACAAGATCGAGCCACACTTTAATACCAGCTGGATGTCCTGGGATGGCTGTGATCTGG
ACTACTATTGCAACATGGGCGACTGGCCCAGCTGCACCTACACAGGCGTGACCCAGCACAATCA
CGCCGCCTTCGTGAACCTGCTGAATATCGAGACAGATTATACCAAGACATTCCACTTTCACTCC
AAGCGCGTGACAGCCCACGGCGATACCCCTCAGCTGGACCTGAAGGCCCGGCCAACATACGGAG
CAGGAGAGATCACCGTGCTGGTGGAGGTGGCCGACATGGAGCTGCACACCAAGAAGGTGGAGAT
CAGCGGCCTGAAGTTTGCCTCTCTGGCCTGCACAGGCTGTTATGCCTGCTCCTCTGGCATCAGC
TGCAAGGTGCGCATCCACGTGGATGAGCCTGACGAGCTGACCGTGCACGTGAAGAGCTCCGATC
CAGACGTGGTGGCAGCATCCACATCTCTGACCGCACGGAAGCTGGAGTTTGGCACAGACAGCAC
CTTCAAGGCCTTTTCCGCCATGCCTAAGACCTCTCTGTGCTTCTACATCGTGGAGAAGGAGTAT
TGTAAGTCTTGCAACGAGGATGACACACAGAAGTGCGTGGATACCAAGCTGGAGCAGCCACAGA
GCATCCTGATCGAGCACAAGGGCACCATCATCGGCAAGCAGAATGACACCTGTACAGCCAAGGC
CTCCTGCTGGCTGGAGTCTGTGAAGAGCTTCTTTTACGGCCTGAAGAACATGCTGGGCAGCGTG
TTCGGCAATTTCTTTATCGGCATCCTGCTGTTTCTGGCCCCCTTCGTGCTGCTGGTGCTGTTCT
TTATGTTTGGCTGGAAGATCCTGTTCTGCTTTAAGTGCTGTAGGCGCACCAGGGGCCTGTTCAA
GTACCGCCACCTGAAGGATGACGAGGAGACAGGCTATAAGCGGATCATCGAGAGACTGAACAAT
AAGAAGGGCAAGAACAGACTGCTGGACGGCGAGAGACTGGCAGACCGGAAAATCGCAGAGCTGT
TTAGTACCAAAACTCACATCGGGTGATGA
SEQ ID NO: 14: CCHF GP-Turkey nucleotide sequence
ATGCCAACTAACATCACCCACACCCTGCTGGTCTGCTTCATCCTGTATCTGCAGCTGCTGGGGA
GAGGCGGCGCACATGGACAGTCAAACGCCACAGAGCACAACGGCACCAATACCACAACCGCACC
AGGCACCTCTCAGAGCCACAAGCCTCTGGTGAGCACAACCCCCCCTCACACACTGGAGAGCTCC
ACCATCAAGCACACAACCCCCACCTCTGAGACAGAGGGAAGCGGAGAGACAACCCCACCACCTA
ACACAACCCAGGGACCTTCCCCACCAGAGGCAACCCCTGAGCGCCCAGCAACAACCGCCACCAG
CACACCCTCCACCGATAACACAAATAGCACAACCCAGATGAATGACAACAATCCTACCTCCACA
ATCTCCACATCTCCCTCTAGCTCCCCTTCTACCCCTCCAACACCTCAGGGCATCCACCACCCAG
CACGGAGCCTGCTGAGCGTGTCTAGCCTGAAGACCGCCACAACCCCAACCCCCACAAGCCCTGG
CGAGATCAGCTCTGAGACAAGCTCCCAGCACTCCGCCATGTCTCGCACCCCAACACTGCACACA
ACCACACAGGTGAGCACCGAGTCCACAAACCACTCCACCCCAAGGCAGTCTGAGTCTAGCGCCC
AGCCTACCACACCTTCCCCAATGACATCTCCAGCCCAGAGCATCCTGCCCATGTCTGCCGCCCC
TACCGCCATCCAGAATATCCACCCCAGCCCTACAAACCGGTCCAAGAGAAATCTGGAGGTGGAG
ATCATCCTGACCCTGTCCCAGGGCCTGAAGAAGTACTATGGCAAGATCCTGAAGCTGCTGCACC
TGACACTGGAGGAGGATACCGAGGGCCTGCTGGAGTGGTGTAAGAGAAACCTGGGCTCCTCTTG
CGACGATGACTTCTTTCAGAAGAGGATCGAGGAGTTCTTTGTGACCGGCGAGGGCTACTTTAAT
GAGGTGCTGCAGTTCAAGACCCTGTCTACACTGAGCCCCACAGAGCCTAGCCACGCCAAGCTGC
CAACCGTGGAGCCCTTCAAGTCCTATTTTGCCAAGGGCTTCCTGTCCATCGACTCTGGCTACTT
TTCCGCCAAGTGTTATCCACGCAGCTCCACATCTGGCCTGCAGCTGATCAACGTGACCCAGCAC
CCAGCAAGGATCGCAGAGACACCAGGACCCAAGACCACATCTCTGAAGACCATCAACTGCATCA
ATCTGAGGGCCAGCGTGTTCAAGGAGCACCGCGAGATCGAGATCAATGTGCTGCTGCCACAGAT
CGCCGTGAACCTGAGCAATTGTCACGCCGTGATCAAGTCTCACGTGTGCGATTACAGCCTGGAT
ACCGACGGCCCTGTGAGACTGCCACACATCTACCACGAGGGCACATTCATCCCCGGCACCTATA
AGATCGTGATCGATAAGAAGAACAAGCTGAATGACAGGTGTATCCTGGTGACCAACTGCGTGAT
CAAGGGAAGGGAGGTGCGCAAGGGACAGTCCGTGCTGAGACAGTATAAGACCGAGATCAAGATC
GGCAAGGCCAGCACAGGCTCCAGGAAGCTGCTGTCCGAGGAGCCTGGCGATGACTGCATCTCTA
GGACCCAGCTGCTGAGGACCGAGACAGCAGAGATCCACGATGACAACTACGGCGGCCCAGGCGA
TAAGATCACAATCTGTAATGGAAGCACCATCGTGGACCAGCGCCTGGGATCCGAGCTGGGCTGC
TATACCATCAACCGGGTGAAGAGCTTTAAGCTGTGCGAGAATTCCGCCACCGGCAAGACATGCG
AGATCGACAGCACCCCTGTGAAGTGTAGACAGGGCTTCTGCCTGAAGATCACACAGGAGGGCCG
GGGCCACGTGAAGCTGTCTAGAGGCAGCGAGGTGGTGCTGGACGTGTGCGACTCTAGCTGCGAA
GTGATGATCCCAAAGGGCACCGGCGATATCCTGGTGGACTGCTCCGGAGGACAGCAGCACTTTC
TGAAGGATAACCTGATCGACCTGGGATGTCCACACGTGCCACTGCTGGGAAGAATGGCCATCTA
CATCTGCCGGATGTCCAATCACCCCAGAACCACAATGGCCTTCCTGTTTTGGTTCTCTTTTGGC
TACGTGATCACCTGCATCTTTTGTAAGGCCCTGTTCTATAGCCTGATCATCATCGGCACACTGG
GCAAGAAGTTCAAGCAGTATAGGGAGCTGAAGCCCCAGACCTGCACAATCTGTGAGACAGCCCC
TGTGAACGCCATCGATGCCGAGATGCACGACCTGAACTGTTCCTACAATATCTGCCCCTATTGT
GCATCCAGGCTGACCTCTGATGGCCTGGCAAGACACGTGCCTCAGTGCCCAAAGAGGAAGGAGA
AGGTGGAGGAGACAGAGCTGTACCTGAATCTGGAGAGGATCCCTTGGATCGTGCGCAAGCTGCT
GCAGGTGAGCGAGTCCACCGGAGTGGCCCTGAAGAGATCCTCTTGGCTGATCGTGCTGCTGGTG
CTGCTGACAGTGTCTCTGAGCCCAGTGCAGAGCGCCCCAGTGGGACACGGCAAGACCATCGAGA
CATATCAGACCAGGGAGGGCTTTACCTCCATCTGTCTGTTCATGCTGGGCTCCATCCTGTTCAT
CGTGTCTTGCCTGGTGAAGGGCCTGGTGGATTCCGTGTCTGACAGCTTCTTTCCCGGCCTGAGC
GTGTGCAAGACCTGTTCCATCGGCTCTATCAACGGCTTTGAGATCGAGAGCCACAAGTGCTACT
GTTCCCTGTTCTGCTGTCCTTATTGCCGGCACTGTTCCGCCGACAGAGAGATCCACCAGCTGCA
CCTGTCTATCTGCAAGAAGAGAAAGACCGGCAGCAACGTGATGCTGGCCGTGTGCAAGAGGATG
TGCTTTCGCGCCACAATCGAGGCCTCTCGGAGAGCCCTGCTGATCAGGAGCATCATCAATACCA
CATTCGTGATCTGTATCCTGACCCTGACAATCTGCGTGGTGTCCACCTCTGCCGTGGAGATGGA
GAATCTGCCAGCAGGCACATGGGAGAGGGAGGAGGATCTGACCAACTTTTGTCACCAGGAGTGC
CAGGTGACCGAGACAGAGTGCCTGTGCCCATACGAGGCCCTGGTGCTGAGGAAGCCTCTGTTCC
TGGACAGCATCGTGAAGGGCATGAAGAACCTGCTGAATAGCACATCCCTGGAGACAAGCCTGAG
CATCGAGGCACCATGGGGAGCCATCAACGTGCAGTCTACCTTTAAGCCCACAGTGAGCACCGCC
AATATCGCCCTGTCCTGGAGCTCCATCGAGCACCGCGGCAACAAGATCCTGGTGACCGGCCGGT
CCGAGTCTATCATGAAGCTGGAGGAGAGGACAGGCGTGAGCTGGGATCTGGGAGTGGAGGACGC
AAGCGAGTCCAAGCTGCTGACCGTGAGCATCATGGACCTGAGCCAGATGTACTCCCCCGTGTTC
GAGTATCTGTCCGGCGATAGACAGGTGGAGGAGTGGCCAAAGGCCACCTGTACAGGCGACTGCC
CCGAGAGGTGCGGCTGCACATCTAGCACCTGTCTGCACAAGGAGTGGCCTCACAGCCGGAACTG
GAGATGTAATCCAACCTGGTGCTGGGGAGTGGGCACAGGATGCACCTGCTGTGGCGTGGATGTG
AAGGACCTGTTTACAGATCACATGTTCGTGAAGTGGAAGGTGGAGTACATCAAGACCGAGGCCA
TCGTGTGCGTGGAGCTGACATCTCAGGAGAGACAGTGCAGCCTGATCGAGGCCGGCACCAGGTT
CAATCTGGGCCCAGTGACCATCACACTGAGCGAGCCCCGCAACATCCAGCAGAAGCTGCCCCCT
GAGATCATCACACTGCACCCAAAGGTGGAGGAGGGCTTCTTTGACCTGATGCACGTGCAGAAGG
TGCTGTCTGCCAGCACCGTGTGCAAGCTGCAGTCCTGCACCCACGGAATCCCAGGCGATCTGCA
GGTGTACCACATCGGCAACCTGCTGAAGGGCGACCGGGTGAATGGCCACCTGATCCACAAGATC
GAGCCACACTTTAATACCAGCTGGATGTCCTGGGATGGCTGTGATCTGGACTACTATTGCAACA
TGGGCGACTGGCCCAGCTGCACCTACACAGGCGTGACCCAGCACAATCACGCCGCCTTCGTGAA
CCTGCTGAATATCGAGACAGATTATACCAAGACATTCCACTTTCACTCCAAGCGCGTGACAGCC
CACGGCGATACCCCTCAGCTGGACCTGAAGGCCCGGCCAACATACGGAGCAGGAGAGATCACCG
TGCTGGTGGAGGTGGCCGACATGGAGCTGCACACCAAGAAGGTGGAGATCAGCGGCCTGAAGTT
TGCCTCTCTGGCCTGCACAGGCTGTTATGCCTGCTCCTCTGGCATCAGCTGCAAGGTGCGCATC
CACGTGGATGAGCCTGACGAGCTGACCGTGCACGTGAAGAGCTCCGATCCAGACGTGGTGGCAG
CATCCACATCTCTGACCGCACGGAAGCTGGAGTTTGGCACAGACAGCACCTTCAAGGCCTTTTC
CGCCATGCCTAAGACCTCTCTGTGCTTCTACATCGTGGAGAAGGAGTATTGTAAGTCTTGCAAC
GAGGATGACACACAGAAGTGCGTGGATACCAAGCTGGAGCAGCCACAGAGCATCCTGATCGAGC
ACAAGGGCACCATCATCGGCAAGCAGAATGACACCTGTACAGCCAAGGCCTCCTGCTGGCTGGA
GTCTGTGAAGAGCTTCTTTTACGGCCTGAAGAACATGCTGGGCAGCGTGTTCGGCAATTTCTTT
ATCGGCATCCTGCTGTTTCTGGCCCCCTTCGTGCTGCTGGTGCTGTTCTTTATGTTTGGCTGGA
AGATCCTGTTCTGCTTTAAGTGCTGTAGGCGCACCAGGGGCCTGTTCAAGTACCGCCACCTGAA
GGATGACGAGGAGACAGGCTATAAGCGGATCATCGAGAGACTGAACAATAAGAAGGGCAAGAAC
AGACTGCTGGACGGCGAGAGACTGGCAGACCGGAAAATCGCAGAGCTGTTTAGTACCAAAACTC
ACATCGGGTGATGA
SEQ ID NO: 15: CCHF GP Turkey amino acid
MPTNITHTLLVCFILYLQLLGRGGAHGQSNATEHNGTNTTTAPGTSQSHKPLVSTTPPHTLESS
TIKHTTPTSETEGSGETTPPPNTTQGPSPPEATPERPATTATSTPSTDNTNSTTQMNDNNPTST
ISTSPSSSPSTPPTPQGIHHPARSLLSVSSLKTATTPTPTSPGEISSETSSQHSAMSRTPTLHT
TTQVSTESTNHSTPRQSESSAQPTTPSPMTSPAQSILPMSAAPTAIQNIHPSPTNRSKRNLEVE
IILTLSQGLKKYYGKILKLLHLTLEEDTEGLLEWCKRNLGSSCDDDFFQKRIEEFFVTGEGYFN
EVLQFKTLSTLSPTEPSHAKLPTVEPFKSYFAKGFLSIDSGYFSAKCYPRSSTSGLQLINVTQH
PARIAETPGPKTTSLKTINCINLRASVFKEHREIEINVLLPQIAVNLSNCHAVIKSHVCDYSLD
TDGPVRLPHIYHEGTFIPGTYKIVIDKKNKLNDRCILVTNCVIKGREVRKGQSVLRQYKTEIKI
GKASTGSRKLLSEEPGDDCISRTQLLRTETAEIHDDNYGGPGDKITICNGSTIVDQRLGSELGC
YTINRVKSFKLCENSATGKTCEIDSTPVKCRQGFCLKITQEGRGHVKLSRGSEVVLDVCDSSCE
VMIPKGTGDILVDCSGGQQHFLKDNLIDLGCPHVPLLGRMAIYICRMSNHPRTTMAFLFWFSFG
YVITCIFCKALFYSLIIIGTLGKKFKQYRELKPQTCTICETAPVNAIDAEMHDLNCSYNICPYC
ASRLTSDGLARHVPQCPKRKEKVEETELYLNLERIPWIVRKLLQVSESTGVALKRSSWLIVLLV
LLTVSLSPVQSAPVGHGKTIETYQTREGFTSICLFMLGSILFIVSCLVKGLVDSVSDSFFPGLS
VCKTCSIGSINGFEIESHKCYCSLFCCPYCRHCSADREIHQLHLSICKKRKTGSNVMLAVCKRM
CFRATIEASRRALLIRSIINTTFVICILTLTICVVSTSAVEMENLPAGTWEREEDLTNFCHQEC
QVTETECLCPYEALVLRKPLFLDSIVKGMKNLLNSTSLETSLSIEAPWGAINVQSTFKPTVSTA
NIALSWSSIEHRGNKILVTGRSESIMKLEERTGVSWDLGVEDASESKLLTVSIMDLSQMYSPVF
EYLSGDRQVEEWPKATCTGDCPERCGCTSSTCLHKEWPHSRNWRCNPTWCWGVGTGCTCCGVDV
KDLFTDHMFVKWKVEYIKTEAIVCVELTSQERQCSLIEAGTRFNLGPVTITLSEPRNIQQKLPP
ElITLHPKVEEGFFDLMHVQKVLSASTVCKLQSCTHGIPGDLQVYHIGNLLKGDRVNGHLIHKI
EPHFNTSWMSWDGCDLDYYCNMGDWPSCTYTGVTQHNHAAFVNLLNIETDYTKTFHFHSKRVTA
HGDTPQLDLKARPTYGAGEITVLVEVADMELHTKKVEISGLKFASLACTGCYACSSGISCKVRI
HVDEPDELTVHVKSSDPDVVAASTSLTARKLEFGTDSTFKAFSAMPKTSLCFYIVEKEYCKSCN
EDDTQKCVDTKLEQPQSILIEHKGTIIGKQNDTCTAKASCWLESVKSFFYGLKNMLGSVFGNFF
IGILLFLAPFVLLVLFFMFGWKILFCFKCCRRTRGLFKYRHLKDDEETGYKRIIERLNNKKGKN
RLLDGERLADRKIAELFSTKTHIG

1. A nucleic acid molecule encoding a Crimean Congo Fever (CCHF) Virus antigen for use in vaccination wherein the nucleic acid molecule comprises a) the sequence set forth in SEQ ID NO:3, SEQ ID NO:4, SEQ ID NO:5, SEQ ID NO:6, in SEQ ID NO:14, b) a sequence at least 70% identical to SEQ ID NO:3, SEQ ID NO:4, SEQ ID NO:5, SEQ ID NO:6, in SEQ ID NO:14 or c) a fragment of a) or b) thereof.

2. A nucleic acid molecule a) having the sequence set forth in SEQ ID NO:3, b) having a sequence at least 70% identical, at least 75% identical, at least 80% identical, at least 85% identical, at least 90% identical, at least 95% identical or at least 99% identical to SEQ ID NO:3 or c) comprising a fragment of a) or b).

3. A nucleic acid molecule a) having the sequence set forth in SEQ ID NO:4, b) having the sequence set forth in nucleotides 229-2163 of SEQ ID NO:4, c) having a sequence at least 70% identical, at least 75% identical, at least 80% identical, at least 85% identical, at least 90% identical, at least 95% identical or at least 99% identical to a) or b) or, d) comprising a fragment of a), b) or c).

4. A nucleic acid molecule a) having the sequence set forth in SEQ ID NO:5, b) having the sequence set forth in nucleotides 229-1092 of SEQ ID NO:5 c) having a sequence at least 70% identical, at least 75% identical, at least 80% identical, at least 85% identical, at least 90% identical, at least 95% identical or at least 99% identical to a) or b) or, d) comprising a fragment of a), b) or c).

5. A nucleic acid molecule a) having the sequence set forth in SEQ ID NO:6, b) having a sequence at least 70% identical, at least 75% identical, at least 80% identical, at least 85% identical, at least 90% identical, at least 95% identical or at least 99% identical to SEQ ID NO:6 or c) comprising a fragment of a) or b).

6. A nucleic acid molecule a) having the sequence set forth in SEQ ID NO:14, b) having a sequence at least 70% identical, at least 75% identical, at least 80% identical, at least 85% identical, at least 90% identical, at least 95% identical or at least 99% identical to SEQ ID NO:14 or c) comprising a fragment of a) or b).

7. The nucleic acid molecule of any one of claims 1 to 6, wherein the nucleic acid encodes a CCHF polypeptide of an infectious CCHF virus, a polypeptide variant or a polypeptide fragment thereof.

8. The nucleic acid molecule of any one of claims 1 to 6, wherein the nucleic acid encodes the polypeptide set forth in SEQ ID NO: 15, a sequence at least 85% identical, at least 90% identical, at least 95% identical or at least 99% identical to SEQ ID NO:15 or a fragment thereof.

9. The nucleic acid molecule of any one of claims 1 to 6, wherein the nucleic acid encodes the polypeptide set forth in SEQ ID NO: 7, a sequence at least 85% identical, at least 90% identical, at least 95% identical or at least 99% identical to SEQ ID NO:7 or a fragment thereof.

10. The nucleic acid molecule of any one of claims 1 to 6, wherein the nucleic acid encodes the polypeptide set forth in amino acid residues 77 to 720 of SEQ ID NO: 8, a sequence at least 85% identical, at least 90% identical, at least 95% identical or at least 99% identical to amino acid residues 77 to 720 of SEQ ID NO: 8 or a fragment thereof.

11. The nucleic acid molecule of any one of claims 1 to 6, wherein the nucleic acid encodes the polypeptide set forth in amino acid residues 77 to 364 of SEQ ID NO: 9, a sequence at least 85% identical, at least 90% identical, at least 95% identical or at least 99% identical to amino acid residues 77 to 364 of SEQ ID NO: 9 or a fragment thereof.

12. The nucleic acid molecule of any one of claims 1 to 6, wherein the nucleic acid encodes the polypeptide set forth in SEQ ID NO:10, a sequence at least 85% identical, at least 90% identical, at least 95% identical or at least 99% identical to SEQ ID NO:10 or a fragment thereof.

13. The nucleic acid molecule of any one of claims 1 to 12, wherein the nucleic acid comprises DNA or RNA.

14. An expression cassette comprising the nucleic acid sequence of any one of claims 1 to 13 and regulatory elements.

15. A vector comprising the nucleic acid sequence of any one of claims 1 to 13 or the expression cassette of claim 14.

16. The vector of claim 15, wherein the vector is a vector for DNA vaccination.

17. The vector of claim 15 or 16, wherein the vector is pVAX.

18. The vector of claim 15 or 16, wherein the vector is pIDV (SEQ ID NO:1).

19. The vector of claim 15 or 16, wherein the vector is pIDV-I (SEQ ID NO:11).

20. The vector of claim 15 or 16, wherein the vector is pIDV-II (SEQ ID NO:12).

21. A set of vectors comprising at least one vector comprising the nucleic acid sequence of any one of claims 1 to 13 or the expression cassette of claim 14 and at least one vector comprising a nucleic acid sequence encoding another antigen.

22. The set of vectors of claim 21, wherein at least one vector comprises a nucleic acid sequence encoding a CCHF glycoprotein and at least one vector comprises a nucleic acid sequence encoding the other antigen.

23. The set of vectors of claim 21 or 22, wherein the other antigen is another CCHF antigen.

24. The vector of claim 15, wherein the vector is a viral vector.

25. The vector of claim 24, wherein the viral vector is from a DNA virus.

26. A vector comprising the nucleic acid sequence set forth in SEQ ID NO:13 or a sequence at least 70% identical, at least 75% identical, at least 80% identical, at least 85% identical, at least 90% identical, at least 95% identical or at least 99% identical.

27. The vector of claim 26, wherein the vector is able to express a transgene.

28. The vector of claim 26 or 27, wherein the vector portion of the vector comprises a sequence at least 70% identical, at least 75% identical, at least 80% identical, at least 85% identical, at least 90% identical, at least 95% identical or at least 99% identical to SEQ ID NO:12.

29. The nucleic acid molecule, vector or set of vectors of any one of claims 1 to 28, wherein the nucleic acid or vector is encapsulated into a nanocarrier.

30. A pharmaceutical composition comprising the nucleic acid molecule, vector or set of vectors of any one of claims 1 to 29 and a pharmaceutically acceptable carrier.

31. A method of immunizing a host, the method comprising administering the pharmaceutical composition of claim 30 to the host.

32. The method of claim 31, wherein the host is a human.

33. The method of claim 31, wherein the host is an animal.

34. The method of any one of claims 31 to 33, wherein the pharmaceutical composition is administered by injection.

35. The method of any one of claims 31 to 33, wherein the pharmaceutical composition is administered by electroporation.

36. The method of any one of claims 31 to 33, wherein the pharmaceutical composition is administered intradermally, transdermally or intramuscularly.

37. The method of any one of claims 31 to 33, wherein the pharmaceutical composition is administered at a mucosal site.

38. A method of manufacturing the vector of any one of claims 15 to 20 or 24 to 28, the method comprising fermenting microorganisms transformed with the vector.

39. The method of claim 38, further comprising isolating substantially purified vectors from the microorganism.

40. A codon-optimized nucleic acid comprising a sequence encoding a Crimean-Congo Hemorrhagic Fever (CCHF) Virus antigen or encoding a fragment thereof, wherein the codon-optimized nucleic acid sequence or fragment thereof comprises a) SEQ ID NO.: 3 or a fragment thereof, b) nucleotides 229-2163 of SEQ ID NO.:4 or a fragment thereof, c) nucleotides 229-1092 of SEQ ID NO.:5 or a fragment thereof, d) SEQ ID NO:14 or a fragment thereof or e) a complement thereof.

41. A DNA vector comprising a nucleic acid sequence encoding a CCHF virus antigen and a vector's backbone having a sequence at least 75% identical, at least 80%, at least 805%, at least 90%, at least 95%, at least 99% identical or identical to SEQ ID NO.:12.

42. A DNA vector comprising a nucleic acid sequence encoding a CCHF glycoprotein or a fragment thereof, wherein the nucleic acid sequence comprises SEQ ID NO.: 3 or a fragment thereof.

43. A DNA vector comprising a nucleic acid sequence encoding a CCHF glycoprotein or a fragment thereof, wherein the nucleic acid sequence comprises nucleotides 229-2163 of SEQ ID NO.:4 or a fragment thereof.

44. A DNA vector comprising a nucleic acid sequence encoding a CCHF glycoprotein or a fragment thereof, wherein the nucleic acid sequence comprises nucleotides 229-1092 of SEQ ID NO.:5 or a fragment thereof.

45. A DNA vector comprising a nucleic acid sequence encoding a CCHF nucleoprotein or a fragment thereof, wherein the nucleic acid sequence comprises SEQ ID NO.:6 or a fragment thereof.

46. A DNA vector comprising a nucleic acid sequence encoding a CCHF glycoprotein or a fragment thereof, wherein the nucleic acid sequence comprises SEQ ID NO.:14 or a fragment thereof.

47. The DNA vector of any one of claims 42 to 46, wherein the vector's backbone comprises a nucleic acid having a sequence at least 75% identical to 100% identical to SEQ ID NO.:1.

48. The DNA vector of any one of claims 42 to 46, wherein the vector's backbone comprises a nucleic acid having a sequence at least 75% identical to 100% identical to SEQ ID NO.:2.

49. The DNA vector of any one of claims 42 to 46, wherein the vector's backbone comprises a nucleic acid having a sequence at least 75% identical to 100% identical to SEQ ID NO.:11.

50. The DNA vector of any one of claims 42 to 46, wherein the vector's backbone comprises a nucleic acid having a sequence at least 75% identical to 100% identical to SEQ ID NO.:12.

51. A DNA vaccine comprising the DNA vector of any one of claims 41 to 50 or a combination thereof.

52. A DNA vaccine comprising the nucleic acid sequence set forth in SEQ ID NO:3.

53. A DNA vaccine comprising the nucleic acid sequence set forth in SEQ ID NO:4 or the nucleic acid sequence set forth in nucleotides 229-2163 of SEQ ID NO:4.

54. A DNA vaccine comprising the nucleic acid sequence set forth in SEQ ID NO:5 or the nucleic acid sequence set forth in nucleotides 229-1092 of SEQ ID NO:5.

55. A DNA vaccine comprising the nucleic acid sequence set forth in SEQ ID NO: 6.

56. A DNA vaccine comprising the nucleic acid sequence set forth in SEQ ID NO:14.

57. A DNA vaccine comprising the nucleic acid sequence set forth in SEQ ID NO:14 and a vector comprising a sequence at least 70% identical, at least 75% identical, at least 80% identical, at least 85% identical, at least 90% identical, at least 95% identical or at least 99% identical to SEQ ID NO: 12.

58. A DNA vaccine comprising the nucleic acid sequence set forth in SEQ ID NO:3 and a vector comprising a sequence at least 70% identical, at least 75% identical, at least 80% identical, at least 85% identical, at least 90% identical, at least 95% identical or at least 99% identical to SEQ ID NO: 12.

59. The DNA vaccine of claim 57 or 58, wherein the vector has a sequence at least 80% identical to SEQ ID NO:12.

60. The DNA vaccine of claim 57 or 58, wherein the vector has a sequence at least 90% identical to SEQ ID NO:12.

61. The DNA vaccine of claim 57 or 58, wherein the vector has a sequence at least 99% identical to SEQ ID NO:12.

62. A DNA vaccine comprising the nucleic acid sequence set forth in SEQ ID NO:13.

63. A DNA vaccine comprising a nucleic acid sequence encoding a CCHF virus antigen expressed by the DNA vector set forth in SEQ ID NO:12.

64. The DNA vector or the DNA vaccine of any one of claims 41 to 63, wherein the CCHF virus antigen comprises the M segment of CCHF.

65. The DNA vector or DNA vaccine of claim 64, wherein the CCHF virus antigen comprises the Gn and/or Gc segment of CCHF.

66. The DNA vector or DNA vaccine of any one of claims 63 to 65, wherein the CCHF virus antigen is from the Kelkit (kk) Turkey 06 strain.

67. The DNA vector or DNA vaccine of any one of claims 63 to 65, wherein the CCHF virus antigen comprises the M segment of Turkey isolate 812955 (Accession number KY362519.1) or a sequence at least 85% identical, at least 90% identical, at least 95% identical, or at least 99% identical.

68. The DNA vector or DNA vaccine of any one of claims 63 to 65, wherein the nucleic acid encodes a CCHF virus antigen having at least 85% identity with the polypeptide set forth in SEQ ID NO: 15 or a fragment thereof.

69. The DNA vector or DNA vaccine of any one of claims 63 to 65, wherein the nucleic acid encodes a CCHF virus antigen having at least 90% identity with the polypeptide set forth in SEQ ID NO: 15 or a fragment thereof.

70. The DNA vector or DNA vaccine of any one of claims 63 to 65, wherein the nucleic acid encodes a CCHF virus antigen having at least 95% identity with the polypeptide set forth in SEQ ID NO: 15 or a fragment thereof.

71. The DNA vector or DNA vaccine of any one of claims 63 to 65, wherein the nucleic acid encodes a CCHF virus antigen having at least 99% identity with the polypeptide set forth in SEQ ID NO: 15 or a fragment thereof.

72. The DNA vector or DNA vaccine of any one of claims 63 to 65, wherein the nucleic acid encodes a CCHF virus antigen is identical to SEQ ID NO:15.

73. The DNA vector or DNA vaccine of any one of claims 63 to 65, wherein the nucleic acid encodes a CCHF virus antigen having at least 85% identity with the polypeptide set forth in SEQ ID NO: 7 or a fragment thereof.

74. The DNA vector or DNA vaccine of any one of claims 63 to 65, wherein the nucleic acid encodes a CCHF virus antigen having at least 90% identity with the polypeptide set forth in SEQ ID NO: 7 or a fragment thereof.

75. The DNA vector or DNA vaccine of any one of claims 63 to 65, wherein the nucleic acid encodes a CCHF virus antigen having at least 95% identity with the polypeptide set forth in SEQ ID NO: 7 or a fragment thereof.

76. The DNA vector or DNA vaccine of any one of claims 63 to 65, wherein the nucleic acid encodes a CCHF virus antigen having at least 99% identity with the polypeptide set forth in SEQ ID NO: 7 or a fragment thereof.

77. The DNA vector or DNA vaccine of any one of claims 63 to 65, wherein the nucleic acid encodes a CCHF virus antigen is identical to SEQ ID NO:7.

78. A pharmaceutical composition comprising the nucleic acid, DNA vector or DNA vaccine of any of the preceding claims.

79. A method of immunizing a host comprising administering the nucleic acid, vector, DNA vaccine, composition or pharmaceutical composition of any of the preceding claims.

80. The method of claim 79, wherein the host is a human.

81. The method of claim 79, wherein the host is an animal.

82. The method of any one of claims 79 to 81, wherein the nucleic acid, vector or DNA vaccine is administered naked.

83. The method of any one of claims 79 to 82, wherein the pharmaceutical composition is administered by injection.

84. The method of any one of claims 79 to 82, wherein the pharmaceutical composition is administered by electroporation.

85. The method of any one of claims 79 to 82, wherein the pharmaceutical composition is administered intradermally, transdermally or intramuscularly.

86. The method of any one of claims 79 to 82, wherein the pharmaceutical composition is administered at a mucosal site.


SEQUENCE LISTING (15 sequences)

SEQ ID NO: 1 - DNA, 4099 bp, Artificial Sequence
  pIDV plasmid nucleotide sequence
agatctttttccctctgccaaaaattatggggacatcatgaagccccttgagcatctgacttctggctaataaaggaaatttattttcattgcaatagtgtgttggaattttttgtgtctctcactcggaaggacatatgggagggcaaatcatttaaaacatcagaatgagtatttggtttagagtttggcaacatatgcccatatgctggctgccatgaacaaaggttggctataaagaggtcatcagtatatgaaacagccccctgctgtccattccttattccatagaaaagccttgacttgaggttagattttttttatattttgttttgtgttatttttttctttaacatccctaaaattttccttacatgttttactagccagatttttcctcctctcctgactactcccagtcatagctgtccctcttctcttatggagatccctcgacctgcagcccaagcttgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatctgtctgacgctcagtggaacgaaaactcacgttaagggattttggtcatgagattatcaaaaaggatcttcacctagatccttttaaattaaaaatgaagttttagcacgtgctattattgaagcatttatcagggttattgtctcatgagcggatacatatttgaatgtatttagaaaaataaacaaataggggttccgcgcacatttccccgaaaagtgccacctgtatgcggtgtgaaataccgcacagatgcgtaaggagaaaataccgcatcaggaaattgtaagcgttaataattcagaagaactcgtcaagaaggcgatagaaggcgatgcgctgcgaatcgggagcggcgataccgtaaagcacgaggaagcggtcagcccattcgccgccaagctcttcagcaatatcacgggtagccaacgctatgtcctgatagcggtccgccacacccagccggccacagtcgatgaatccagaaaagcggccattttccaccatgatattcggcaagcaggcatcgccatgggtcacgacgagatcctcgccgtcgggcatgctcgccttgagcctggcgaacagttcggctggcgcgagcccctgatgctcttcgtccagatcatcctgatcgacaagaccggcttccatccgagtacgtgctcgctcgatgcgatgtttcgcttggtggtcgaatgggcaggtagccggatcaagcgtatgcagccgccgcattgcatcagccatgatggatactttctcggcaggagcaaggtgagatgacaggagatcctgccccggcacttcgcccaatagcagccagtcccttcccgcttcagtgacaacgtcgagcacagctgcgcaaggaacgcccgtcgtggccagccacgatagccgcgctgcctcgtcttgcagttcattcagggcaccggacaggtcggtcttgacaaaaagaaccgggcgcccctgcgctgacagccggaacacggcggcatcagagcagccgattgtctgttgtgcccagtcatagccgaatagcctctccacccaagcggccggagaacctgcgtgcaatccatcttgttcaatcatgcgaaacgatcctcatcctgtctcttgatcagagcttgatcccctgcgccatcagatccttggcggcgagaaagccatccagtttactttgcagggcttcccaaccttaccagagggcgccccagctggcaattccggttcgcttgctgtccataaaaccgcccagtagaaggcatgcctgctactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcgggggggggggggccgcgcgccagccggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgttgccttcgccccgtgccccgctccgcgccgcctcgcgccgcccgccccggctctgactgaccgcgttactcccacaggtgagcgggcgggacggcccttctcctccgggctgtaattagcgcttggtttaatgacggctcgtttcttttctgtggctgcgtgaaagccttaaagggctccgggagggccctttgtgcggggggagcggctcggggggtgcgtgcgtgtgtgtgtgcgtggggagcgccgcgtgcggctccgcgctgcccggcggctgtgagcgctgcgggcgcggcgcggggctttgtgcgctccgcagtgtgcgcgaggggagcgcggccgggggcggtgccccgcggtgcggggggggctgcgaggggaacaaaggctgcgtgcggggtgtgtgcgtgggggggtgagcagggggtgtgggcgcgtcggtcgggctgcaaccccccctgcacccccctccccgagttgctgagcacggcccggcttcgggtgcggggctccgtacggggcgtggcgcggggctcgccgtgccgggcggggggtggcggcaggtgggggtgccgggcggggcggggccgcctcgggccggggagggctcgggggaggggcgcggcggcccccggagcgccggcggctgtcgaggcgcggcgagccgcagccattgccttttatggtaatcgtgcgagagggcgcagggacttcctttgtcccaaatctgtgcggagccgaaatctgggaggcgccgccgcaccccctctagcgggcgcggggcgaagcggtgcggcgccggcaggaaggaaatgggcggggagggccttcgtgcgtcgccgcgccgccgtccccttctccctctccagcctcggggctgtccgcggggggacggctgccttcgggggggacggggcagggcggggttcggcttctggcgtgtgaccggcggctctagagcctctgctaaccatgttcatgccttcttctttttcctacagctcctgggcaacgtgctggttattgtgctgtctcatcattttggcaaagaattcgagctcatcgatgcatggtacc

SEQ ID NO: 2 - DNA, 2999 bp, Artificial Sequence
  pVAX1 plasmid sequence
gactcttcgcgatgtacgggccagatatacgcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggactatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctctggctaactagagaacccactgcttactggcttatcgaaattaatacgactcactatagggagacccaagctggctagcgtttaaacttaagcttggtaccgagctcggatccactagtccagtgtggtggaattctgcagatatccagcacagtggcggccgctcgagtctagagggcccgtttaaacccgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctactgggcggttttatggacagcaagcgaaccggaattgccagctggggcgccctctggtaaggttgggaagccctgcaaagtaaactggatggctttctcgccgccaaggatctgatggcgcaggggatcaagctctgatcaagagacaggatgaggatcgtttcgcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaagacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgagcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgaattattaacgcttacaatttcctgatgcggtattttctccttacgcatctgtgcggtatttcacaccgcatacaggtggcacttttcggggaaatgtgcgcggaacccctatttgtttatttttctaaatacattcaaatatgtatccgctcatgagacaataaccctgataaatgcttcaataatagcacgtgctaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctgggcttttgctggccttttgctcacatgttctt

SEQ ID NO: 3 - DNA, 5067 bp, Artificial Sequence
  Codon-optimized CCHF GP-Turkey nucleotide sequence
atgcctaccaatatcatgcatacacccctcgtgtgctttatactttacctccaattgttgtgcttgggtggggcccacggacaattgaacgccaccgaacacaatgggacgaacaataccactgctcccggcgctagtcaatctcctaaacctcccatgagcaccacgcctccacatgcgccagaatcatcaacaatcaagcccacgacacctatctccgaggcggaggggtcaggagagactacgtcacccccgaataccacgcagggcctgtcttctccggaaaccacttccgaaaggccagcaactacgagcattagtactagcagtaccgattccacgaacccaacgacacaaatgacggacaatactcctacgccaacagttagtacatcccccagctccagtccttcaacccctagtactccgcagggcatccaccatccagcgagatccctcctgtctgtcagtagcccaaagactgtcacgacaccaacgccgacctctcccggagagatgtcttctgagacttcttcacagcatagcgcgatgtcaagaatcccgactccccacacagcgacgcgcgtttcaacagaaatcacaaaccaccggactccgcgacaatctgagtcatctgctcaacaaactactccttctcctatgacgtctcctgcccagtccattttgcttatgagcgcagctccaactgcggtccaggacatccacccttcccctactaataggtccaagcgaaaccttgagacagaaattattctcacactctcccaggggttgaaaaaatattacggcaaaatcctgaaactgttgcatctgaccctcgagcaagacactgaaggcctcctcgagtggtgtaagggtaatttgggcagcaattgtgatgatgatttcttccaaaaaaggatcgaggaattctttatgaccggcgagtgctattttaatgaggtccttcagttcaagacactgagtaccctctcacctaccgaaccttcacacgccaggcttccgacagccgaaccttttaaaagttacttcgcaaagggctttttgagtatcgactccggatacttcagtgctaagtgctaccctcggtcaagcgcatccgggctccagttgattaatgttacacagcatcccgcacggatcgcagagacaccaggtccgaaaactacttccttgaagacaatcaactgcattaacctgcgagcttccgtcttcaaagagcatagagaagtcgaaataaatgtacttctcccccagatcgcagttaacttgtcaaattgtcacgtcgtaatcaacagccatgtttgtgactattcccttgataccgatggtcccgttcgactccctaggatataccacgaaggaacattcataccgggcacttataagatcgtaatcgataagaagaataaacttaatgaccggtgtacgctggtcaccaattgcgtgatcaaaggtagagaggtgagaaaaggacaaagtgtgttgcgacaatataagacggaaatcaaaattggcaaggcttccacggggttcagaaaactgctcagtgaggagccgggcgacgactgcatctccaggacgcaacttttgagaacagagacagccgagattcatgatgataattatggaggacctggagacaaaataacgatatgtaacggctccactatagtcgaccaaagattgggaagcgagcttggttgctacacgattaacagagtgaagtcttttaaactttgtaagaacagtgccacggggaagacatgtgaagttgactccaccccagtgaaatgccgacaaggattttgtctgaagattacacaagaaggtcgaggccacgtcaaactttcaaggggaagtgaagtcgttttggacgcttgcgattcatcctgcgaagtaatgattccaaaaggtactggagacattttggtggattgttcaggcgggcaacaacactttctgaaagacaacctgatagacctcggatgccctcatatccccctgcttggtaggatggcaatctatatctgtcggatgtcaaatcatcctcgaactacgatggcattcctcttttggttctcttttggttacgtcattacatgcattttctgtaaggcactcttttattcactcattatcatcgggacacttggaaaaaaaatcaaacaataccgggaacttaagccccaaacctgcaccatttgcgaaactgcgccagtaaacgcaatcgatgccgaaatgcatgatctcaattgttcatacaacatatgtccctactgtgcgagcaggcttactagtgatggtttggcacgacacgtaactcagtgtcctaagagaaaagaaaaagtcgaagaaacagaattgtacctcaacctcgaacgaataccttggatcgtaaggaagctcctgcaggtctcagagagtaccggggtcgctcttaaaagaagctcatggctgatcgtacttttggtcctcctcactgtatcactgtcccctgtccaaagcgccccagtcggacatggcaaaactattgagatatatcaaacgcgagaagggtttgcatctatttgtctttttatgctcggcagcatcctcttcatagtgtcttgcttggtcaagggactggtagacagcgtgtctgaatctttctttcctgggctctcagtctgtaagacttgttccattggcagtgtcaacggtttcgagattgaatcccacaagtgttattgcagtttgttttgctgcccatactgccgccattgtagcgcggatcgagaaattcatcaactgcaccttagcatttgcaaaaagaggaaaacgggctcaaacgtaatgctcgcggtctgcaaacgaatgtgtttcagagcgacaattgaggcaagtcggcgagcactgctgattaggagtattattaatactacgtttgtgatctgtatcctcacgctgactatatgcgttgtcagtacatccgccgtcgagatggaaaatcttcctgcagggacgtgggagcgagaggaagatcttactaatttttgccatcaagaatgtcaagtgacagaaactgaatgcttgtgcccctatgaagcccttgtgttgaggaagccactctttcttgattctatagttaagggaatgaaaaatttgctcaacagtacgagtctggaaacctctttgtcaatcgaggccccatggggtgccatcaacgtgcagagtacgtttaagccaaccgtttcaaccgctaacatcgctcttagctggtcaagcgtcgttcatagaggaaacaagatacttgtcactggacggagtgaaagcatcatgaagcttgaggaaaggactggggtgagttgggatcttggggtcgaggacgcatctgaaagtaaactgttgacggtctctatcatggatctctcacagatgtacagcccggtttttgagtacctcagcggggatcgacaagttgaggagtggcccaaggctacctgcaccggagattgcccggagcgatgcgggtgcactagctcaacatgtctccataaggagtggtcccatagccgaaattggaggtgtaaccccacttggtgctggggtgtcggcacaggatgtacttgttgcggagtcgatgtaaaagacctgttcacggaccatatgtttgtcaaatggaaggtggagtacattaaaactgaagccattgtgtgcgttgcgcttacgtctcaagaacggcaatgttcactgatcgaagcagggactcggttcaacttggggcctgtcacaataactttgtcagagccgagaaacatacaacagaagcttccacccgaaatcataaccttgcatccgaaaatagaggaagggttcttcgatcttatgcacgtccaaaaagtgcttagtgcctctacggtctgcaaactccaaagctgcactcacggtatcccgggggatctgcaagtttaccacataggcaaccttttgaaaggagatagggtcaacggccacctgatacataagattgaaagccatttcaacacaagttggatgagttgggatggctgcgatttggattattactgcaatatgggggactggcccagctgtacatatactggagttacacagcataatcatgcggcatttgttaacctccttaacatcgagacagactacacaaagacctttcacttccactccaagagagtgactgcgcacggagacacgccccaacttgaccttaaagctagaccgacctacggagcgggcggaatcacagtcctggttgaagtagctgatatggaattgcatacgaaaaaggttgagattagtgggctcaagttcgcgtcactcgcttgcacgggttgctatgcgtgttccagcggcattagctgcaaggtcagaatccatgttgacgagccggatgagttgacagtacatgtcaaatccagtgacccagacgttgtcgctgcaagtacatccctgatggcgaggaagctggaattcggcacggactccacgttcaaagcgttttcagcgatgccgaagacgtctttgtgtttctatattgtagagcgagaatattgtaaaagctgcagtgaagatgacactcagaaatgcgttgatactcgcttggaacagcctcagtcaattctcatagaacataagggaacaattatcggaaaacagaacgatacttgtaccgccaaagcatcctgttggctggaaagtgtgaagtcttttttctatggcctgaaaaacatgcttgggagtgtcttcgggaatttgttcatcggcatactgcttttcttggccccctttgtcctcctcgtcctcttcttcatgttcggatggaagatactgttttgcttcaaatgctgcagacgcactagggggctgttcaagtatcgacatctgaaggacgacgaagagaccgggtaccgaaggattatcgagagactcaattctaaaaagggcaaaaatcggttgcttgacggggaccgcttggcagacaggaagatcgcagagttgttctccacgaaaacacatatcggataa

SEQ ID NO: 4 - DNA, 2163 bp, Artificial Sequence
  Ubiquitin-CCHF Gycoprotein GC fusion codon-optimized nucleotide sequence
atgcagatcttcgtgaaaacccttaccggcaagaccatcacccttgaggtggagcccagtgacaccatcgaaaatgtgaaggccaagatccaggataaggaaggcattccccccgaccagcagaggctcatctttgcaggcaagcagctggaagatggccgtactctttctgactacaacatccagaaggagtcgaccctgcacctggtcctgcgtctgagaggtggttttcttgattctatagttaagggaatgaaaaatttgctcaacagtacgagtctggaaacctctttgtcaatcgaggccccatggggtgccatcaacgtgcagagtacgtttaagccaaccgtttcaaccgctaacatcgctcttagctggtcaagcgtcgttcatagaggaaacaagatacttgtcactggacggagtgaaagcatcatgaagcttgaggaaaggactggggtgagttgggatcttggggtcgaggacgcatctgaaagtaaactgttgacggtctctatcatggatctctcacagatgtacagcccggtttttgagtacctcagcggggatcgacaagttgaggagtggcccaaggctacctgcaccggagattgcccggagcgatgcgggtgcactagctcaacatgtctccataaggagtggtcccatagccgaaattggaggtgtaaccccacttggtgctggggtgtcggcacaggatgtacttgttgcggagtcgatgtaaaagacctgttcacggaccatatgtttgtcaaatggaaggtggagtacattaaaactgaagccattgtgtgcgttgcgcttacgtctcaagaacggcaatgttcactgatcgaagcagggactcggttcaacttggggcctgtcacaataactttgtcagagccgagaaacatacaacagaagcttccacccgaaatcataaccttgcatccgaaaatagaggaagggttcttcgatcttatgcacgtccaaaaagtgcttagtgcctctacggtctgcaaactccaaagctgcactcacggtatcccgggggatctgcaagtttaccacataggcaaccttttgaaaggagatagggtcaacggccacctgatacataagattgaaagccatttcaacacaagttggatgagttgggatggctgcgatttggattattactgcaatatgggggactggcccagctgtacatatactggagttacacagcataatcatgcggcatttgttaacctccttaacatcgagacagactacacaaagacctttcacttccactccaagagagtgactgcgcacggagacacgccccaacttgaccttaaagctagaccgacctacggagcgggcggaatcacagtcctggttgaagtagctgatatggaattgcatacgaaaaaggttgagattagtgggctcaagttcgcgtcactcgcttgcacgggttgctatgcgtgttccagcggcattagctgcaaggtcagaatccatgttgacgagccggatgagttgacagtacatgtcaaatccagtgacccagacgttgtcgctgcaagtacatccctgatggcgaggaagctggaattcggcacggactccacgttcaaagcgttttcagcgatgccgaagacgtctttgtgtttctatattgtagagcgagaatattgtaaaagctgcagtgaagatgacactcagaaatgcgttgatactcgcttggaacagcctcagtcaattctcatagaacataagggaacaattatcggaaaacagaacgatacttgtaccgccaaagcatcctgttggctggaaagtgtgaagtcttttttctatggcctgaaaaacatgcttgggagtgtcttcgggaatttgttcatcggcatactgcttttcttggccccctttgtcctcctcgtcctcttcttcatgttcggatggaagatactgttttgcttcaaatgctgcagacgcactagggggctgttcaagtatcgacatctgaaggacgacgaagagaccgggtaccgaaggattatcgagagactcaattctaaaaagggcaaaaatcggttgcttgacggggaccgcttggcagacaggaagatcgcagagttgttctccacgaaaacacatatcggataa

SEQ ID NO: 5 - DNA, 1092 bp, Artificial Sequence
  Ubiquitin-CCHF Glycoprotein Gn fusion codon-optimized nucleotide sequence
atgcagatcttcgtgaaaacccttaccggcaagaccatcacccttgaggtggagcccagtgacaccatcgaaaatgtgaaggccaagatccaggataaggaaggcattccccccgaccagcagaggctcatctttgcaggcaagcagctggaagatggccgtactctttctgactacaacatccagaaggagtcgaccctgcacctggtcctgcgtctgagaggtggtagtgaggagccgggcgacgactgcatctccaggacgcaacttttgagaacagagacagccgagattcatgatgataattatggaggacctggagacaaaataacgatatgtaacggctccactatagtcgaccaaagattgggaagcgagcttggttgctacacgattaacagagtgaagtcttttaaactttgtaagaacagtgccacggggaagacatgtgaagttgactccaccccagtgaaatgccgacaaggattttgtctgaagattacacaagaaggtcgaggccacgtcaaactttcaaggggaagtgaagtcgttttggacgcttgcgattcatcctgcgaagtaatgattccaaaaggtactggagacattttggtggattgttcaggcgggcaacaacactttctgaaagacaacctgatagacctcggatgccctcatatccccctgcttggtaggatggcaatctatatctgtcggatgtcaaatcatcctcgaactacgatggcattcctcttttggttctcttttggttacgtcattacatgcattttctgtaaggcactcttttattcactcattatcatcgggacacttggaaaaaaaatcaaacaataccgggaacttaagccccaaacctgcaccatttgcgaaactgcgccagtaaacgcaatcgatgccgaaatgcatgatctcaattgttcatacaacatatgtccctactgtgcgagcaggcttactagtgatggtttggcacgacacgtaactcagtgtcctaagagaaaagaaaaagtcgaagaaacagaattgtacctcaacctcgaacgaataccttggatcgtaaggaagctcctg

SEQ ID NO: 6 - DNA, 1449 bp, Crimean-Congo hemorrhagic fever virus
atggaaaacaagatcgaggtgaataacaaagatgagatgaacaggtggtttgaagagttcaaaaaaggaaatggacttgtggacaccttcacaaactcctattccttttgcgagagtgttcccaatttggacaggtttgtgtttcagatggccagtgccaccgatgatgcacagaaggactccatctacgcatctgctctggtggaggcaacaaagttttgtgcacctatatatgagtgcgcatgggttagctccactggcattgtaaaaaagggacttgaatggttcgagaaaaatgcaggaaccattaagtcctgggatgaaagttatactgagctaaaggtcgacgtcccgaaaatagagcagcttaccggttaccaacaagctgccttgaagtggagaaaagacataggtttccgtgtcaatgccaacacagcagctctgagcaacaaagtcctcgcagaatacaaagtccctggtgagattgtgatgtctgtcaaagagatgctgtcagacatgattaggagaaggaacctgattctaaacaggggtggtgatgagaacccacgtggcccagtgagccatgagcatgtagactggtgcagggagtttgtcaaaggcaaatacatcatggccttcaacccaccatggggggacatcaacaagtcaggccgttcaggaatagcacttgttgcaacaggccttgctaagcttgcagagactgaaggaaagggaatatttgatgaagccaaaaagactgtggaggccctcaacgggtatctggacaagcataaggacgaagttgatagagcaagcgccgacagcatgataacaaaccttcttaagcatattgccaaggcacaggagctctataaaaattcatctgcacttcgtgcacaaagcgcacagattgacactgctttcagctcatactattggctttacaaggctggcgtgactcctgaaaccttcccgacggtgtcacagttcctctttgagctagggaaacagccaagaggtaccaagaaaatgaagaaggctcttctgagcaccccaatgaagtgggggaagaagctttatgagctctttgccgatgattctttccagcagaacaggatttacatgcatcctgccgtgcttacagctggtagaatcagtgaaatgggagtctgctttgggacaatccctgtggccaatcctgatgatgctgcccaaggatctggacacactaagtctattctcaacctccgtaccaacactgagaccaataatccgtgtgccaaaaccatcgtcaagctatttgaagttcaaaaaacagggttcaacattcaggacatggacatagtggcctctgagcacttgctacaccaatcccttgttggcaagcaatccccattccagaacgcctacaacgtcaagggcaatgccaccagtgctaacatcatttaa

SEQ ID NO: 7 - protein, 1688 aa, Crimean-Congo hemorrhagic fever virus
MPTNIMHTPLVCFILYLQLLCLGGAHGQLNATEHNGTNNTTAPGASQSPKPPMSTTPPHAPESSTIKPTTPISEAEGSGETTSPPNTTQGLSSPETTSERPATTSISTSSTDSTNPTTQMTDNTPTPTVSTSPSSSPSTPSTPQGIHHPARSLLSVSSPKTVTTPTPTSPGEMSSETSSQHSAMSRIPTPHTATRVSTEITNHRTPRQSESSAQQTTPSPMTSPAQSILLMSAAPTAVQDIHPSPTNRSKRNLETEIILTLSQGLKKYYGKILKLLHLTLEQDTEGLLEWCKGNLGSNCDDDFFQKRIEEFFMTGECYFNEVLQFKTLSTLSPTEPSHARLPTAEPFKSYFAKGFLSIDSGYFSAKCYPRSSASGLQLINVTQHPARIAETPGPKTTSLKTINCINLRASVFKEHREVEINVLLPQIAVNLSNCHVVINSHVCDYSLDTDGPVRLPRIYHEGTFIPGTYKIVIDKKNKLNDRCTLVTNCVIKGREVRKGQSVLRQYKTEIKIGKASTGFRKLLSEEPGDDCISRTQLLRTETAEIHDDNYGGPGDKITICNGSTIVDQRLGSELGCYTINRVKSFKLCKNSATGKTCEVDSTPVKCRQGFCLKITQEGRGHVKLSRGSEVVLDACDSSCEVMIPKGTGDILVDCSGGQQHFLKDNLIDLGCPHIPLLGRMAIYICRMSNHPRTTMAFLFWFSFGYVITCIFCKALFYSLIIIGTLGKKIKQYRELKPQTCTICETAPVNAIDAEMHDLNCSYNICPYCASRLTSDGLARHVTQCPKRKEKVEETELYLNLERIPWIVRKLLQVSESTGVALKRSSWLIVLLVLLTVSLSPVQSAPVGHGKTIEIYQTREGFASICLFMLGSILFIVSCLVKGLVDSVSESFFPGLSVCKTCSIGSVNGFEIESHKCYCSLFCCPYCRHCSADREIHQLHLSICKKRKTGSNVMLAVCKRMCFRATIEASRRALLIRSIINTTFVICILTLTICVVSTSAVEMENLPAGTWEREEDLTNFCHQECQVTETECLCPYEALVLRKPLFLDSIVKGMKNLLNSTSLETSLSIEAPWGAINVQSTFKPTVSTANIALSWSSVVHRGNKILVTGRSESIMKLEERTGVSWDLGVEDASESKLLTVSIMDLSQMYSPVFEYLSGDRQVEEWPKATCTGDCPERCGCTSSTCLHKEWSHSRNWRCNPTWCWGVGTGCTCCGVDVKDLFTDHMFVKWKVEYIKTEAIVCVALTSQERQCSLIEAGTRFNLGPVTITLSEPRNIQQKLPPEIITLHPKIEEGFFDLMHVQKVLSASTVCKLQSCTHGIPGDLQVYHIGNLLKGDRVNGHLIHKIESHFNTSWMSWDGCDLDYYCNMGDWPSCTYTGVTQHNHAAFVNLLNIETDYTKTFHFHSKRVTAHGDTPQLDLKARPTYGAGGITVLVEVADMELHTKKVEISGLKFASLACTGCYACSSGISCKVRIHVDEPDELTVHVKSSDPDVVAASTSLMARKLEFGTDSTFKAFSAMPKTSLCFYIVEREYCKSCSEDDTQKCVDTRLEQPQSILIEHKGTIIGKQNDTCTAKASCWLESVKSFFYGLKNMLGSVFGNLFIGILLFLAPFVLLVLFFMFGWKILFCFKCCRRTRGLFKYRHLKDDEETGYRRIIERLNSKKGKNRLLDGDRLADRKIAELFSTKTHIG

SEQ ID NO: 8 - protein, 720 aa, Artificial Sequence
  Ubiquitin CCHF Glycoprotein GC fusion amino acid sequence
MQIFVKTLTGKTITLEVEPSDTIENVKAKIQDKEGIPPDQQRLIFAGKQLEDGRTLSDYNIQKESTLHLVLRLRGGFLDSIVKGMKNLLNSTSLETSLSIEAPWGAINVQSTFKPTVSTANIALSWSSVVHRGNKILVTGRSESIMKLEERTGVSWDLGVEDASESKLLTVSIMDLSQMYSPVFEYLSGDRQVEEWPKATCTGDCPERCGCTSSTCLHKEWSHSRNWRCNPTWCWGVGTGCTCCGVDVKDLFTDHMFVKWKVEYIKTEAIVCVALTSQERQCSLIEAGTRFNLGPVTITLSEPRNIQQKLPPEIITLHPKIEEGFFDLMHVQKVLSASTVCKLQSCTHGIPGDLQVYHIGNLLKGDRVNGHLIHKIESHFNTSWMSWDGCDLDYYCNMGDWPSCTYTGVTQHNHAAFVNLLNIETDYTKTFHFHSKRVTAHGDTPQLDLKARPTYGAGGITVLVEVADMELHTKKVEISGLKFASLACTGCYACSSGISCKVRIHVDEPDELTVHVKSSDPDVVAASTSLMARKLEFGTDSTFKAFSAMPKTSLCFYIVEREYCKSCSEDDTQKCVDTRLEQPQSILIEHKGTIIGKQNDTCTAKASCWLESVKSFFYGLKNMLGSVFGNLFIGILLFLAPFVLLVLFFMFGWKILFCFKCCRRTRGLFKYRHLKDDEETGYRRIIERLNSKKGKNRLLDGDRLADRKIAELFSTKTHIG

SEQ ID NO: 9 - protein, 364 aa, Artificial Sequence
  Ubiquitin CCHF Glycoprotein Gn fusion amino acid sequence
MQIFVKTLTGKTITLEVEPSDTIENVKAKIQDKEGIPPDQQRLIFAGKQLEDGRTLSDYNIQKESTLHLVLRLRGGSEEPGDDCISRTQLLRTETAEIHDDNYGGPGDKITICNGSTIVDQRLGSELGCYTINRVKSFKLCKNSATGKTCEVDSTPVKCRQGFCLKITQEGRGHVKLSRGSEVVLDACDSSCEVMIPKGTGDILVDCSGGQQHFLKDNLIDLGCPHIPLLGRMAIYICRMSNHPRTTMAFLFWFSFGYVITCIFCKALFYSLIIIGTLGKKIKQYRELKPQTCTICETAPVNAIDAEMHDLNCSYNICPYCASRLTSDGLARHVTQCPKRKEKVEETELYLNLERIPWIVRKLL

SEQ ID NO: 10 - protein, 482 aa, Crimean-Congo hemorrhagic fever virus
MENKIEVNNKDEMNRWFEEFKKGNGLVDTFTNSYSFCESVPNLDRFVFQMASATDDAQKDSIYASALVEATKFCAPIYECAWVSSTGIVKKGLEWFEKNAGTIKSWDESYTELKVDVPKIEQLTGYQQAALKWRKDIGFRVNANTAALSNKVLAEYKVPGEIVMSVKEMLSDMIRRRNLILNRGGDENPRGPVSHEHVDWCREFVKGKYIMAFNPPWGDINKSGRSGIALVATGLAKLAETEGKGIFDEAKKTVEALNGYLDKHKDEVDRASADSMITNLLKHIAKAQELYKNSSALRAQSAQIDTAFSSYYWLYKAGVTPETFPTVSQFLFELGKQPRGTKKMKKALLSTPMKWGKKLYELFADDSFQQNRIYMHPAVLTAGRISEMGVCFGTIPVANPDDAAQGSGHTKSILNLRTNTETNNPCAKTIVKLFEVQKTGFNIQDMDIVASEHLLHQSLVGKQSPFQNAYNVKGNATSANII

SEQ ID NO: 11 - DNA, 4029 bp, Artificial Sequence
  pIDV-I plasmid nucleotide sequence
agatcttttttccctctgccaaaaattatggggacatcatgaagccccttgagcatctgacttctggctaataaaggaaatttattttcattgcaatagtgtgttggaattttttgtgtctctcactcggaaggacatatgggagggcaaatcatttaaaacatcagaatgagtatttggtttagagtttggcaacatatgcccatatgctggctgccatgaacaaaggttggctataaagaggtcatcagtatatgaaacagccccctgctgtccattccttattccatagaaaagccttgacttgaggttagattttttttatattttgttttgtgttatttttttctttaacatccctaaaattttccttacatgttttactagccagatttttcctcctctcctgactactcccagtcatagctgtccctcttctcttatggagatccctcgacctgcagcccaagcttgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatctgtctgacgctcagtggaacgaaaactcacgttaagggattttggtcatgagattatcaaaaaggatcttcacctagatccttttaaattaaaaatgaagttttagcacgtgctattattgaagcacacatttccccgaaaagtgccacctgtatgcggtgtgaaataccgcacagatgcgtaaggagaaaataccgcatcaggaaattgtaagcgttaataattcagaagaactcgtcaagaaggcgatagaaggcgatgcgctgcgaatcgggagcggcgataccgtaaagcacgaggaagcggtcagcccattcgccgccaagctcttcagcaatatcacgggtagccaacgctatgtcctgatagcggtccgccacacccagccggccacagtcgatgaatccagaaaagcggccattttccaccatgatattcggcaagcaggcatcgccatgggtcacgacgagatcctcgccgtcgggcatgctcgccttgagcctggcgaacagttcggctggcgcgagcccctgatgctcttcgtccagatcatcctgatcgacaagaccggcttccatccgagtacgtgctcgctcgatgcgatgtttcgcttggtggtcgaatgggcaggtagccggatcaagcgtatgcagccgccgcattgcatcagccatgatggatactttctcggcaggagcaaggtgagatgacaggagatcctgccccggcacttcgcccaatagcagccagtcccttcccgcttcagtgacaacgtcgagcacagctgcgcaaggaacgcccgtcgtggccagccacgatagccgcgctgcctcgtcttgcagttcattcagggcaccggacaggtcggtcttgacaaaaagaaccgggcgcccctgcgctgacagccggaacacggcggcatcagagcagccgattgtctgttgtgcccagtcatagccgaatagcctctccacccaagcggccggagaacctgcgtgcaatccatcttgttcaatcatgcgaaacgatcctcatcctgtctcttgatcagagcttgatcccctgcgccatcagatccttggcggcgagaaagccatccagtttactttgcagggcttcccaaccttaccagagggcgccccagctggcaattccggttcgcttgctgtccataaaaccgcccagtagaaggcatgcctgctactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgcgctgccttcgccccgtgccccgctccgccgccgcctcgcgccgcccgccccggctctgactgaccgcgttactcccacaggtgagcgggcgggacggcccttctcctccgggctgtaattagcgcttggtttaatgacggctcgtttcttttctgtggctgcgtgaaagccttaaagggctccgggagggccctttgtgcggggggagcggctcggggggtgcgtgcgtgtgtgtgtgcgtggggagcgccgcgtgcggctccgcgctgcccggcggctgtgagcgctgcgggcgcggcgcggggctttgtgcgctccgcagtgtgcgcgaggggagcgcggccgggggcggtgccccgcggtgcggggggggctgcgaggggaacaaaggctgcgtgcggggtgtgtgcgtgggggggtgagcagggggtgtgggcgcgtcggtcgggctgcaaccccccctgcacccccctccccgagttgctgagcacggcccggcttcgggtgcggggctccgtacggggcgtggcgcggggctcgccgtgccgggcggggggtggcggcaggtgggggtgccgggcggggcggggccgcctcgggccggggagggctcgggggaggggcgcggcggcccccggagcgccggcggctgtcgaggcgcggcgagccgcagccattgccttttatggtaatcgtgcgagagggcgcagggacttcctttgtcccaaatctgtgcggagccgaaatctgggaggcgccgccgcaccccctctagcgggcgcggggcgaagcggtgcggcgccggcaggaaggaaatgggcggggagggccttcgtgcgtcgccgcgccgccgtccccttctccctctccagcctcggggctgtccgcggggggacggctgccttcgggggggacggggcagggcggggttcggcttctggcgtgtgaccggcggctctagagcctctgctaaccatgttcatgccttcttctttttcctacagctcctgggcaacgtgctggttattgtgctgtctcatcattttggcaaagaattcgagctcatcgatgcatggtacc

SEQ ID NO: 12 - DNA, 4618 bp, Artificial Sequence
  pIDV-II plasmid nucleotide sequence
agatctaatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaagctgacgtcctttccatggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgctttttccctctgccaaaaattatggggacatcatgaagccccttgagcatctgacttctggctaataaaggaaatttattttcattgcaatagtgtgttggaattttttgtgtctctcactcggaaggacatatgggagggcaaatcatttaaaacatcagaatgagtatttggtttagagtttggcaacatatgcccatatgctggctgccatgaacaaaggttggctataaagaggtcatcagtatatgaaacagccccctgctgtccattccttattccatagaaaagccttgacttgaggttagattttttttatattttgttttgtgttatttttttctttaacatccctaaaattttccttacatgttttactagccagatttttcctcctctcctgactactcccagtcatagctgtccctcttctcttatggagatccctcgacctgcagcccaagcttgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatctgtctgacgctcagtggaacgaaaactcacgttaagggattttggtcatgagattatcaaaaaggatcttcacctagatccttttaaattaaaaatgaagttttagcacgtgctattattgaagcacacatttccccgaaaagtgccacctgtatgcggtgtgaaataccgcacagatgcgtaaggagaaaataccgcatcaggaaattgtaagcgttaataattcagaagaactcgtcaagaaggcgatagaaggcgatgcgctgcgaatcgggagcggcgataccgtaaagcacgaggaagcggtcagcccattcgccgccaagctcttcagcaatatcacgggtagccaacgctatgtcctgatagcggtccgccacacccagccggccacagtcgatgaatccagaaaagcggccattttccaccatgatattcggcaagcaggcatcgccatgggtcacgacgagatcctcgccgtcgggcatgctcgccttgagcctggcgaacagttcggctggcgcgagcccctgatgctcttcgtccagatcatcctgatcgacaagaccggcttccatccgagtacgtgctcgctcgatgcgatgtttcgcttggtggtcgaatgggcaggtagccggatcaagcgtatgcagccgccgcattgcatcagccatgatggatactttctcggcaggagcaaggtgagatgacaggagatcctgccccggcacttcgcccaatagcagccagtcccttcccgcttcagtgacaacgtcgagcacagctgcgcaaggaacgcccgtcgtggccagccacgatagccgcgctgcctcgtcttgcagttcattcagggcaccggacaggtcggtcttgacaaaaagaaccgggcgcccctgcgctgacagccggaacacggcggcatcagagcagccgattgtctgttgtgcccagtcatagccgaatagcctctccacccaagcggccggagaacctgcgtgcaatccatcttgttcaatcatgcgaaacgatcctcatcctgtctcttgatcagagcttgatcccctgcgccatcagatccttggcggcgagaaagccatccagtttactttgcagggcttcccaaccttaccagagggcgccccagctggcaattccggttcgcttgctgtccataaaaccgcccagtagaaggcatgcctgctactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgcgctgccttcgccccgtgccccgctccgccgccgcctcgcgccgcccgccccggctctgactgaccgcgttactcccacaggtgagcgggcgggacggcccttctcctccgggctgtaattagcgcttggtttaatgacggctcgtttcttttctgtggctgcgtgaaagccttaaagggctccgggagggccctttgtgcggggggagcggctcggggggtgcgtgcgtgtgtgtgtgcgtggggagcgccgcgtgcggctccgcgctgcccggcggctgtgagcgctgcgggcgcggcgcggggctttgtgcgctccgcagtgtgcgcgaggggagcgcggccgggggcggtgccccgcggtgcggggggggctgcgaggggaacaaaggctgcgtgcggggtgtgtgcgtgggggggtgagcagggggtgtgggcgcgtcggtcgggctgcaaccccccctgcacccccctccccgagttgctgagcacggcccggcttcgggtgcggggctccgtacggggcgtggcgcggggctcgccgtgccgggcggggggtggcggcaggtgggggtgccgggcggggcggggccgcctcgggccggggagggctcgggggaggggcgcggcggcccccggagcgccggcggctgtcgaggcgcggcgagccgcagccattgccttttatggtaatcgtgcgagagggcgcagggacttcctttgtcccaaatctgtgcggagccgaaatctgggaggcgccgccgcaccccctctagcgggcgcggggcgaagcggtgcggcgccggcaggaaggaaatgggcggggagggccttcgtgcgtcgccgcgccgccgtccccttctccctctccagcctcggggctgtccgcggggggacggctgccttcgggggggacggggcagggcggggttcggcttctggcgtgtgaccggcggctctagagcctctgctaaccatgttcatgccttcttctttttcctacagctcctgggcaacgtgctggttattgtgctgtctcatcattttggcaaagaattcgagctcatcgatgcatggtacc

SEQ ID NO: 13 - DNA, 9693 bp, Artificial Sequence
  pIDV-II-CCHF-GP-Turkey nucleotide sequence
taatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaagctgacgtcctttccatggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgctttttccctctgccaaaaattatggggacatcatgaagccccttgagcatctgacttctggctaataaaggaaatttattttcattgcaatagtgtgttggaattttttgtgtctctcactcggaaggacatatgggagggcaaatcatttaaaacatcagaatgagtatttggtttagagtttggcaacatatgcccatatgctggctgccatgaacaaaggttggctataaagaggtcatcagtatatgaaacagccccctgctgtccattccttattccatagaaaagccttgacttgaggttagattttttttatattttgttttgtgttatttttttctttaacatccctaaaattttccttacatgttttactagccagatttttcctcctctcctgactactcccagtcatagctgtccctcttctcttatggagatccctcgacctgcagcccaagcttgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatctgtctgacgctcagtggaacgaaaactcacgttaagggattttggtcatgagattatcaaaaaggatcttcacctagatccttttaaattaaaaatgaagttttagcacgtgctattattgaagcacacatttccccgaaaagtgccacctgtatgcggtgtgaaataccgcacagatgcgtaaggagaaaataccgcatcaggaaattgtaagcgttaataattcagaagaactcgtcaagaaggcgatagaaggcgatgcgctgcgaatcgggagcggcgataccgtaaagcacgaggaagcggtcagcccattcgccgccaagctcttcagcaatatcacgggtagccaacgctatgtcctgatagcggtccgccacacccagccggccacagtcgatgaatccagaaaagcggccattttccaccatgatattcggcaagcaggcatcgccatgggtcacgacgagatcctcgccgtcgggcatgctcgccttgagcctggcgaacagttcggctggcgcgagcccctgatgctcttcgtccagatcatcctgatcgacaagaccggcttccatccgagtacgtgctcgctcgatgcgatgtttcgcttggtggtcgaatgggcaggtagccggatcaagcgtatgcagccgccgcattgcatcagccatgatggatactttctcggcaggagcaaggtgagatgacaggagatcctgccccggcacttcgcccaatagcagccagtcccttcccgcttcagtgacaacgtcgagcacagctgcgcaaggaacgcccgtcgtggccagccacgatagccgcgctgcctcgtcttgcagttcattcagggcaccggacaggtcggtcttgacaaaaagaaccgggcgcccctgcgctgacagccggaacacggcggcatcagagcagccgattgtctgttgtgcccagtcatagccgaatagcctctccacccaagcggccggagaacctgcgtgcaatccatcttgttcaatcatgcgaaacgatcctcatcctgtctcttgatcagagcttgatcccctgcgccatcagatccttggcggcgagaaagccatccagtttactttgcagggcttcccaaccttaccagagggcgccccagctggcaattccggttcgcttgctgtccataaaaccgcccagtagaaggcatgcctgctactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgcgctgccttcgccccgtgccccgctccgccgccgcctcgcgccgcccgccccggctctgactgaccgcgttactcccacaggtgagcgggcgggacggcccttctcctccgggctgtaattagcgcttggtttaatgacggctcgtttcttttctgtggctgcgtgaaagccttaaagggctccgggagggccctttgtgcggggggagcggctcggggggtgcgtgcgtgtgtgtgtgcgtggggagcgccgcgtgcggctccgcgctgcccggcggctgtgagcgctgcgggcgcggcgcggggctttgtgcgctccgcagtgtgcgcgaggggagcgcggccgggggcggtgccccgcggtgcggggggggctgcgaggggaacaaaggctgcgtgcggggtgtgtgcgtgggggggtgagcagggggtgtgggcgcgtcggtcgggctgcaaccccccctgcacccccctccccgagttgctgagcacggcccggcttcgggtgcggggctccgtacggggcgtggcgcggggctcgccgtgccgggcggggggtggcggcaggtgggggtgccgggcggggcggggccgcctcgggccggggagggctcgggggaggggcgcggcggcccccggagcgccggcggctgtcgaggcgcggcgagccgcagccattgccttttatggtaatcgtgcgagagggcgcagggacttcctttgtcccaaatctgtgcggagccgaaatctgggaggcgccgccgcaccccctctagcgggcgcggggcgaagcggtgcggcgccggcaggaaggaaatgggcggggagggccttcgtgcgtcgccgcgccgccgtccccttctccctctccagcctcggggctgtccgcggggggacggctgccttcgggggggacggggcagggcggggttcggcttctggcgtgtgaccggcggctctagagcctctgctaaccatgttcatgccttcttctttttcctacagctcctgggcaacgtgctggttattgtgctgtctcatcattttggcaaagaattcgagctcatcgatgcatggtacggtaccgcaccatgccaactaacatcacccacaccctgctggtctgcttcatcctgtatctgcagctgctggggagaggcggcgcacatggacagtcaaacgccacagagcacaacggcaccaataccacaaccgcaccaggcacctctcagagccacaagcctctggtgagcacaaccccccctcacacactggagagctccaccatcaagcacacaacccccacctctgagacagagggaagcggagagacaaccccaccacctaacacaacccagggaccttccccaccagaggcaacccctgagcgcccagcaacaaccgccaccagcacaccctccaccgataacacaaatagcacaacccagatgaatgacaacaatcctacctccacaatctccacatctccctctagctccccttctacccctccaacacctcagggcatccaccacccagcacggagcctgctgagcgtgtctagcctgaagaccgccacaaccccaacccccacaagccctggcgagatcagctctgagacaagctcccagcactccgccatgtctcgcaccccaacactgcacacaaccacacaggtgagcaccgagtccacaaaccactccaccccaaggcagtctgagtctagcgcccagcctaccacaccttccccaatgacatctccagcccagagcatcctgcccatgtctgccgcccctaccgccatccagaatatccaccccagccctacaaaccggtccaagagaaatctggaggtggagatcatcctgaccctgtcccagggcctgaagaagtactatggcaagatcctgaagctgctgcacctgacactggaggaggataccgagggcctgctggagtggtgtaagagaaacctgggctcctcttgcgacgatgacttctttcagaagaggatcgaggagttctttgtgaccggcgagggctactttaatgaggtgctgcagttcaagaccctgtctacactgagccccacagagcctagccacgccaagctgccaaccgtggagcccttcaagtcctattttgccaagggcttcctgtccatcgactctggctacttttccgccaagtgttatccacgcagctccacatctggcctgcagctgatcaacgtgacccagcacccagcaaggatcgcagagacaccaggacccaagaccacatctctgaagaccatcaactgcatcaatctgagggccagcgtgttcaaggagcaccgcgagatcgagatcaatgtgctgctgccacagatcgccgtgaacctgagcaattgtcacgccgtgatcaagtctcacgtgtgcgattacagcctggataccgacggccctgtgagactgccacacatctaccacgagggcacattcatccccggcacctataagatcgtgatcgataagaagaacaagctgaatgacaggtgtatcctggtgaccaactgcgtgatcaagggaagggaggtgcgcaagggacagtccgtgctgagacagtataagaccgagatcaagatcggcaaggccagcacaggctccaggaagctgctgtccgaggagcctggcgatgactgcatctctaggacccagctgctgaggaccgagacagcagagatccacgatgacaactacggcggcccaggcgataagatcacaatctgtaatggaagcaccatcgtggaccagcgcctgggatccgagctgggctgctataccatcaaccgggtgaagagctttaagctgtgcgagaattccgccaccggcaagacatgcgagatcgacagcacccctgtgaagtgtagacagggcttctgcctgaagatcacacaggagggccggggccacgtgaagctgtctagaggcagcgaggtggtgctggacgtgtgcgactctagctgcgaagtgatgatcccaaagggcaccggcgatatcctggtggactgctccggaggacagcagcactttctgaaggataacctgatcgacctgggatgtccacacgtgccactgctgggaagaatggccatctacatctgccggatgtccaatcaccccagaaccacaatggccttcctgttttggttctcttttggctacgtgatcacctgcatcttttgtaaggccctgttctatagcctgatcatcatcggcacactgggcaagaagttcaagcagtatagggagctgaagccccagacctgcacaatctgtgagacagcccctgtgaacgccatcgatgccgagatgcacgacctgaactgttcctacaatatctgcccctattgtgcatccaggctgacctctgatggcctggcaagacacgtgcctcagtgcccaaagaggaaggagaaggtggaggagacagagctgtacctgaatctggagaggatcccttggatcgtgcgcaagctgctgcaggtgagcgagtccaccggagtggccctgaagagatcctcttggctgatcgtgctgctggtgctgctgacagtgtctctgagcccagtgcagagcgccccagtgggacacggcaagaccatcgagacatatcagaccagggagggctttacctccatctgtctgttcatgctgggctccatcctgttcatcgtgtcttgcctggtgaagggcctggtggattccgtgtctgacagcttctttcccggcctgagcgtgtgcaagacctgttccatcggctctatcaacggctttgagatcgagagccacaagtgctactgttccctgttctgctgtccttattgccggcactgttccgccgacagagagatccaccagctgcacctgtctatctgcaagaagagaaagaccggcagcaacgtgatgctggccgtgtgcaagaggatgtgctttcgcgccacaatcgaggcctctcggagagccctgctgatcaggagcatcatcaataccacattcgtgatctgtatcctgaccctgacaatctgcgtggtgtccacctctgccgtggagatggagaatctgccagcaggcacatgggagagggaggaggatctgaccaacttttgtcaccaggagtgccaggtgaccgagacagagtgcctgtgcccatacgaggccctggtgctgaggaagcctctgttcctggacagcatcgtgaagggcatgaagaacctgctgaatagcacatccctggagacaagcctgagcatcgaggcaccatggggagccatcaacgtgcagtctacctttaagcccacagtgagcaccgccaatatcgccctgtcctggagctccatcgagcaccgcggcaacaagatcctggtgaccggccggtccgagtctatcatgaagctggaggagaggacaggcgtgagctgggatctgggagtggaggacgcaagcgagtccaagctgctgaccgtgagcatcatggacctgagccagatgtactcccccgtgttcgagtatctgtccggcgatagacaggtggaggagtggccaaaggccacctgtacaggcgactgccccgagaggtgcggctgcacatctagcacctgtctgcacaaggagtggcctcacagccggaactggagatgtaatccaacctggtgctggggagtgggcacaggatgcacctgctgtggcgtggatgtgaaggacctgtttacagatcacatgttcgtgaagtggaaggtggagtacatcaagaccgaggccatcgtgtgcgtggagctgacatctcaggagagacagtgcagcctgatcgaggccggcaccaggttcaatctgggcccagtgaccatcacactgagcgagccccgcaacatccagcagaagctgccccctgagatcatcacactgcacccaaaggtggaggagggcttctttgacctgatgcacgtgcagaaggtgctgtctgccagcaccgtgtgcaagctgcagtcctgcacccacggaatcccaggcgatctgcaggtgtaccacatcggcaacctgctgaagggcgaccgggtgaatggccacctgatccacaagatcgagccacactttaataccagctggatgtcctgggatggctgtgatctggactactattgcaacatgggcgactggcccagctgcacctacacaggcgtgacccagcacaatcacgccgccttcgtgaacctgctgaatatcgagacagattataccaagacattccactttcactccaagcgcgtgacagcccacggcgatacccctcagctggacctgaaggcccggccaacatacggagcaggagagatcaccgtgctggtggaggtggccgacatggagctgcacaccaagaaggtggagatcagcggcctgaagtttgcctctctggcctgcacaggctgttatgcctgctcctctggcatcagctgcaaggtgcgcatccacgtggatgagcctgacgagctgaccgtgcacgtgaagagctccgatccagacgtggtggcagcatccacatctctgaccgcacggaagctggagtttggcacagacagcaccttcaaggccttttccgccatgcctaagacctctctgtgcttctacatcgtggagaaggagtattgtaagtcttgcaacgaggatgacacacagaagtgcgtggataccaagctggagcagccacagagcatcctgatcgagcacaagggcaccatcatcggcaagcagaatgacacctgtacagccaaggcctcctgctggctggagtctgtgaagagcttcttttacggcctgaagaacatgctgggcagcgtgttcggcaatttctttatcggcatcctgctgtttctggcccccttcgtgctgctggtgctgttctttatgtttggctggaagatcctgttctgctttaagtgctgtaggcgcaccaggggcctgttcaagtaccgccacctgaaggatgacgaggagacaggctataagcggatcatcgagagactgaacaataagaagggcaagaacagactgctggacggcgagagactggcagaccggaaaatcgcagagctgtttagtaccaaaactcacatcgggtgatga

SEQ ID NO: 14 - DNA, 5070 bp, Artificial Sequence
  Codon-optimized CCHF GP-Turkey nucleotide sequence
atgccaactaacatcacccacaccctgctggtctgcttcatcctgtatctgcagctgctggggagaggcggcgcacatggacagtcaaacgccacagagcacaacggcaccaataccacaaccgcaccaggcacctctcagagccacaagcctctggtgagcacaaccccccctcacacactggagagctccaccatcaagcacacaacccccacctctgagacagagggaagcggagagacaaccccaccacctaacacaacccagggaccttccccaccagaggcaacccctgagcgcccagcaacaaccgccaccagcacaccctccaccgataacacaaatagcacaacccagatgaatgacaacaatcctacctccacaatctccacatctccctctagctccccttctacccctccaacacctcagggcatccaccacccagcacggagcctgctgagcgtgtctagcctgaagaccgccacaaccccaacccccacaagccctggcgagatcagctctgagacaagctcccagcactccgccatgtctcgcaccccaacactgcacacaaccacacaggtgagcaccgagtccacaaaccactccaccccaaggcagtctgagtctagcgcccagcctaccacaccttccccaatgacatctccagcccagagcatcctgcccatgtctgccgcccctaccgccatccagaatatccaccccagccctacaaaccggtccaagagaaatctggaggtggagatcatcctgaccctgtcccagggcctgaagaagtactatggcaagatcctgaagctgctgcacctgacactggaggaggataccgagggcctgctggagtggtgtaagagaaacctgggctcctcttgcgacgatgacttctttcagaagaggatcgaggagttctttgtgaccggcgagggctactttaatgaggtgctgcagttcaagaccctgtctacactgagccccacagagcctagccacgccaagctgccaaccgtggagcccttcaagtcctattttgccaagggcttcctgtccatcgactctggctacttttccgccaagtgttatccacgcagctccacatctggcctgcagctgatcaacgtgacccagcacccagcaaggatcgcagagacaccaggacccaagaccacatctctgaagaccatcaactgcatcaatctgagggccagcgtgttcaaggagcaccgcgagatcgagatcaatgtgctgctgccacagatcgccgtgaacctgagcaattgtcacgccgtgatcaagtctcacgtgtgcgattacagcctggataccgacggccctgtgagactgccacacatctaccacgagggcacattcatccccggcacctataagatcgtgatcgataagaagaacaagctgaatgacaggtgtatcctggtgaccaactgcgtgatcaagggaagggaggtgcgcaagggacagtccgtgctgagacagtataagaccgagatcaagatcggcaaggccagcacaggctccaggaagctgctgtccgaggagcctggcgatgactgcatctctaggacccagctgctgaggaccgagacagcagagatccacgatgacaactacggcggcccaggcgataagatcacaatctgtaatggaagcaccatcgtggaccagcgcctgggatccgagctgggctgctataccatcaaccgggtgaagagctttaagctgtgcgagaattccgccaccggcaagacatgcgagatcgacagcacccctgtgaagtgtagacagggcttctgcctgaagatcacacaggagggccggggccacgtgaagctgtctagaggcagcgaggtggtgctggacgtgtgcgactctagctgcgaagtgatgatcccaaagggcaccggcgatatcctggtggactgctccggaggacagcagcactttctgaaggataacctgatcgacctgggatgtccacacgtgccactgctgggaagaatggccatctacatctgccggatgtccaatcaccccagaaccacaatggccttcctgttttggttctcttttggctacgtgatcacctgcatcttttgtaaggccctgttctatagcctgatcatcatcggcacactgggcaagaagttcaagcagtatagggagctgaagccccagacctgcacaatctgtgagacagcccctgtgaacgccatcgatgccgagatgcacgacctgaactgttcctacaatatctgcccctattgtgcatccaggctgacctctgatggcctggcaagacacgtgcctcagtgcccaaagaggaaggagaaggtggaggagacagagctgtacctgaatctggagaggatcccttggatcgtgcgcaagctgctgcaggtgagcgagtccaccggagtggccctgaagagatcctcttggctgatcgtgctgctggtgctgctgacagtgtctctgagcccagtgcagagcgccccagtgggacacggcaagaccatcgagacatatcagaccagggagggctttacctccatctgtctgttcatgctgggctccatcctgttcatcgtgtcttgcctggtgaagggcctggtggattccgtgtctgacagcttctttcccggcctgagcgtgtgcaagacctgttccatcggctctatcaacggctttgagatcgagagccacaagtgctactgttccctgttctgctgtccttattgccggcactgttccgccgacagagagatccaccagctgcacctgtctatctgcaagaagagaaagaccggcagcaacgtgatgctggccgtgtgcaagaggatgtgctttcgcgccacaatcgaggcctctcggagagccctgctgatcaggagcatcatcaataccacattcgtgatctgtatcctgaccctgacaatctgcgtggtgtccacctctgccgtggagatggagaatctgccagcaggcacatgggagagggaggaggatctgaccaacttttgtcaccaggagtgccaggtgaccgagacagagtgcctgtgcccatacgaggccctggtgctgaggaagcctctgttcctggacagcatcgtgaagggcatgaagaacctgctgaatagcacatccctggagacaagcctgagcatcgaggcaccatggggagccatcaacgtgcagtctacctttaagcccacagtgagcaccgccaatatcgccctgtcctggagctccatcgagcaccgcggcaacaagatcctggtgaccggccggtccgagtctatcatgaagctggaggagaggacaggcgtgagctgggatctgggagtggaggacgcaagcgagtccaagctgctgaccgtgagcatcatggacctgagccagatgtactcccccgtgttcgagtatctgtccggcgatagacaggtggaggagtggccaaaggccacctgtacaggcgactgccccgagaggtgcggctgcacatctagcacctgtctgcacaaggagtggcctcacagccggaactggagatgtaatccaacctggtgctggggagtgggcacaggatgcacctgctgtggcgtggatgtgaaggacctgtttacagatcacatgttcgtgaagtggaaggtggagtacatcaagaccgaggccatcgtgtgcgtggagctgacatctcaggagagacagtgcagcctgatcgaggccggcaccaggttcaatctgggcccagtgaccatcacactgagcgagccccgcaacatccagcagaagctgccccctgagatcatcacactgcacccaaaggtggaggagggcttctttgacctgatgcacgtgcagaaggtgctgtctgccagcaccgtgtgcaagctgcagtcctgcacccacggaatcccaggcgatctgcaggtgtaccacatcggcaacctgctgaagggcgaccgggtgaatggccacctgatccacaagatcgagccacactttaataccagctggatgtcctgggatggctgtgatctggactactattgcaacatgggcgactggcccagctgcacctacacaggcgtgacccagcacaatcacgccgccttcgtgaacctgctgaatatcgagacagattataccaagacattccactttcactccaagcgcgtgacagcccacggcgatacccctcagctggacctgaaggcccggccaacatacggagcaggagagatcaccgtgctggtggaggtggccgacatggagctgcacaccaagaaggtggagatcagcggcctgaagtttgcctctctggcctgcacaggctgttatgcctgctcctctggcatcagctgcaaggtgcgcatccacgtggatgagcctgacgagctgaccgtgcacgtgaagagctccgatccagacgtggtggcagcatccacatctctgaccgcacggaagctggagtttggcacagacagcaccttcaaggccttttccgccatgcctaagacctctctgtgcttctacatcgtggagaaggagtattgtaagtcttgcaacgaggatgacacacagaagtgcgtggataccaagctggagcagccacagagcatcctgatcgagcacaagggcaccatcatcggcaagcagaatgacacctgtacagccaaggcctcctgctggctggagtctgtgaagagcttcttttacggcctgaagaacatgctgggcagcgtgttcggcaatttctttatcggcatcctgctgtttctggcccccttcgtgctgctggtgctgttctttatgtttggctggaagatcctgttctgctttaagtgctgtaggcgcaccaggggcctgttcaagtaccgccacctgaaggatgacgaggagacaggctataagcggatcatcgagagactgaacaataagaagggcaagaacagactgctggacggcgagagactggcagaccggaaaatcgcagagctgtttagtaccaaaactcacatcgggtgatga

SEQ ID NO: 15 - protein, 1694 aa, Crimean-Congo hemorrhagic fever virus
CDNCDNMPTNITHTLLVCFILYLQLLGRGGAHGQSNATEHNGTNTTTAPGTSQSHKPLVSTTPPHTLESSTIKHTTPTSETEGSGETTPPPNTTQGPSPPEATPERPATTATSTPSTDNTNSTTQMNDNNPTSTISTSPSSSPSTPPTPQGIHHPARSLLSVSSLKTATTPTPTSPGEISSETSSQHSAMSRTPTLHTTTQVSTESTNHSTPRQSESSAQPTTPSPMTSPAQSILPMSAAPTAIQNIHPSPTNRSKRNLEVEIILTLSQGLKKYYGKILKLLHLTLEEDTEGLLEWCKRNLGSSCDDDFFQKRIEEFFVTGEGYFNEVLQFKTLSTLSPTEPSHAKLPTVEPFKSYFAKGFLSIDSGYFSAKCYPRSSTSGLQLINVTQHPARIAETPGPKTTSLKTINCINLRASVFKEHREIEINVLLPQIAVNLSNCHAVIKSHVCDYSLDTDGPVRLPHIYHEGTFIPGTYKIVIDKKNKLNDRCILVTNCVIKGREVRKGQSVLRQYKTEIKIGKASTGSRKLLSEEPGDDCISRTQLLRTETAEIHDDNYGGPGDKITICNGSTIVDQRLGSELGCYTINRVKSFKLCENSATGKTCEIDSTPVKCRQGFCLKITQEGRGHVKLSRGSEVVLDVCDSSCEVMIPKGTGDILVDCSGGQQHFLKDNLIDLGCPHVPLLGRMAIYICRMSNHPRTTMAFLFWFSFGYVITCIFCKALFYSLIIIGTLGKKFKQYRELKPQTCTICETAPVNAIDAEMHDLNCSYNICPYCASRLTSDGLARHVPQCPKRKEKVEETELYLNLERIPWIVRKLLQVSESTGVALKRSSWLIVLLVLLTVSLSPVQSAPVGHGKTIETYQTREGFTSICLFMLGSILFIVSCLVKGLVDSVSDSFFPGLSVCKTCSIGSINGFEIESHKCYCSLFCCPYCRHCSADREIHQLHLSICKKRKTGSNVMLAVCKRMCFRATIEASRRALLIRSIINTTFVICILTLTICVVSTSAVEMENLPAGTWEREEDLTNFCHQECQVTETECLCPYEALVLRKPLFLDSIVKGMKNLLNSTSLETSLSIEAPWGAINVQSTFKPTVSTANIALSWSSIEHRGNKILVTGRSESIMKLEERTGVSWDLGVEDASESKLLTVSIMDLSQMYSPVFEYLSGDRQVEEWPKATCTGDCPERCGCTSSTCLHKEWPHSRNWRCNPTWCWGVGTGCTCCGVDVKDLFTDHMFVKWKVEYIKTEAIVCVELTSQERQCSLIEAGTRFNLGPVTITLSEPRNIQQKLPPEIITLHPKVEEGFFDLMHVQKVLSASTVCKLQSCTHGIPGDLQVYHIGNLLKGDRVNGHLIHKIEPHFNTSWMSWDGCDLDYYCNMGDWPSCTYTGVTQHNHAAFVNLLNIETDYTKTFHFHSKRVTAHGDTPQLDLKARPTYGAGEITVLVEVADMELHTKKVEISGLKFASLACTGCYACSSGISCKVRIHVDEPDELTVHVKSSDPDVVAASTSLTARKLEFGTDSTFKAFSAMPKTSLCFYIVEKEYCKSCNEDDTQKCVDTKLEQPQSILIEHKGTIIGKQNDTCTAKASCWLESVKSFFYGLKNMLGSVFGNFFIGILLFLAPFVLLVLFFMFGWKILFCFKCCRRTRGLFKYRHLKDDEETGYKRIIERLNNKKGKNRLLDGERLADRKIAELFSTKTHIG